(19) United States
(12) Patent Application Publication
(10) Pub. No.: US 2024/0216518 A1
(43) Pub. Date: Jul. 4, 2024

(54) EXTENDED-RELEASE IMMUNE CELL ENGAGING PROTEINS AND METHODS OF TREATMENT

(71) Applicant: Harpoon Therapeutics, Inc., South San Francisco, CA (US)

(72) Inventors: Holger WESCHE, San Francisco, CA (US); Shuoyen Jack LIN, San Bruno, CA (US); Kathryn KWANT, San Bruno, CA (US); Bryan D. LEMON, Mountain View, CA (US); Richard J. AUSTIN, San Francisco, CA (US)

(21) Appl. No.: 18/525,574

(22) Filed: Nov. 30, 2023

Related U.S. Application Data
(63) Continuation of application No. PCT/US2022/034856, filed on Jun. 24, 2022.
(60) Provisional application No. 63/328,603, filed on Apr. 7, 2022.
(60) Provisional application No. 63/276,804, filed on Nov. 8, 2021.
(60) Provisional application No. 63/214,963, filed on Jun. 25, 2021.

(51) Int. Cl. A61K 47/64; A61P 35/00

(52) U.S. Cl. CPC A61K 47/64; A61P 35/00

(57) ABSTRACT

Provided herein are descriptions of extended-release immune cell binding protein comprising a masking peptide and cleavable linker, and the methods of using thereof. Also described is a pharmaceutical composition comprising a protein with a masking peptide and a cleavable linker, and the method of using thereof.

### CROSS-REFERENCE

This application is a continuation application of International Application NO: PCT/US2022/034856, filed Jun. 24, 2022; and claims the benefit of U.S. Provisional Patent Application No. 63/214,963, filed Jun. 25, 2021; U.S. Provisional Patent Application No. 63/276,804, filed Nov. 8, 2021; and U.S. Provisional Patent Application No. 63/328,603, filed Apr. 7, 2022; each of which are incorporated herein by reference in their entirety.

### SEQUENCE LISTING

The instant application contains a Sequence Listing which has been submitted electronically in XML file format and is hereby incorporated by reference in its entirety. Said XML copy, created on Feb. 13, 2024, is named 47517-754_301_SL.xml and is 5,107,733 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Cancer is the second leading cause of human death next to coronary disease. Worldwide, millions of people die from cancer every year. In the United States alone, cancer causes the death of well over a half-million people each year, with some 1.4 million new cases diagnosed per year. While deaths from heart disease have been declining significantly, those resulting from cancer generally are on the rise. In the early part of the next century, cancer is predicted to become the leading cause of death.

Moreover, even for those cancer patients that initially survive their primary cancers, common experience has shown that their lives are dramatically altered. Many cancer patients experience strong anxieties driven by the awareness of the potential for recurrence or treatment failure. Many cancer patients experience significant physical debilitations following treatment.

Generally speaking, the fundamental problem in the management of the deadliest cancers is the lack of effective and non-toxic systemic therapies. Cancer is a complex disease characterized by genetic mutations that lead to uncontrolled cell growth. Cancerous cells are present in all organisms and, under normal circumstances, their excessive growth is tightly regulated by various physiological factors. Controlling the toxicity of various cancer therapies remain an important and unsolved issue.

### SUMMARY OF THE INVENTION

Provided here in is a pharmaceutical composition comprising an extended-release binding protein which comprises a half-life extended immune cell engaging protein, a masking peptide, and a cleavable linker, wherein the masking peptide is covalently linked to the N-terminus or the C-terminus of the half-life-extended immune cell engaging protein through the cleavable linker, and wherein the cleavable linker is significantly cleaved in systemic circulation. In some embodiments, the extended-release binding protein has a higher therapeutic index than a corresponding half-life-extended immune cell engaging protein without the masking peptide. In some embodiments, administration of the extended-release binding protein results in a lower Cmax/Cmin ratio of an active version of the extended-release binding protein in systemic circulation than the Cmax/Cmin ratio when a corresponding half-life extended immune cell engaging protein without the masking peptide is administered. In some embodiments, multiple administration of the extended-release binding protein results in a more gradual increase of a level of an active version of the extended-release binding protein in systemic circulation than when a corresponding half-life-extended immune cell engaging protein without the masking peptide is administered. In some embodiments, administration of the extended-release binding protein results in a lower Cytokine release syndrome (CRS) level than the CRS observed when a corresponding half-life extended immune cell engaging protein without the masking peptide is administered.

In some embodiments, the half-life extended immune cell engaging protein comprises an immune cell engaging domain. In some embodiments, the immune cell engaging domain comprises a natural killer (NK) cell engaging domain, a T cell engaging domain, a NK-T cell engaging domain, a B cell engaging domain, a dendritic cell engaging domain, a macrophage cell engaging domain, or a combination thereof. In some embodiments, the immune cell engaging domain comprises the T cell engaging domain. In some embodiments, the T cell engaging domain binds a CD3 molecule. In some embodiments, the CD3 molecule is at least one of: a CD3γ molecule, a CD3δ molecule, or a CD3ε molecule.

In some embodiments, the half-life extended immune cell engaging protein comprises a first domain (A), a second domain (B), and a third domain (C), wherein (i) the first domain (A) is the T cell engaging domain and specifically binds to human CD3, (ii) the second domain (B) specifically binds to human serum albumin (HSA), and (iii) the third domain (C) specifically binds to a target antigen; and wherein the domains are linked in one of the following orders: H₂N-(C)-(B)-(A)-COOH, H₂N-(A)-(B)-(C)-COOH, H₂N-(B)-(A)-(C)-COOH, H₂N-(C)-(A)-(B)-COOH, H₂N-(A)-(C)-(B)-COOH, H₂N-(B)-(C)-(A)-COOH, or by linkers L1 and L2 in one of the following orders: H₂N-(C)-L1-(B)-L2(A)-COOH, H₂N-(A)-L1-(B)-L2-(C)-COOH, H₂N-(B)-L1-(A)-L2-(C)-COOH, H₂N-(C)-L1-(A)-L2-(B)-COOH, H₂N-(A)-L1-(C)-L2(B)-COOH, H₂N-(B)-L1-(C)-L2-(A)-COOH. In some embodiments, the third domain comprises a single domain antibody (sdAb), a single-chain variable fragment (scFv), a variable heavy domain (VH), a variable light domain (VL), antigen-binding fragment (Fab), a DARPin or a peptide. In some embodiments, the masking peptide inhibits or reduces the binding of the first domain (A) to the human CD3. In some embodiments, the masking peptide inhibits or reduces the binding of the first domain (A) to the N-terminus of human CD3. In some embodiments, the masking peptide comprises an amino acid sequence having at least 80% homology to QDGNEE (SEQ ID NO: 3068). In some embodiments, the masking peptide comprises an amino acid sequence selected from the group consisting of SEQ ID NOS: 3068 and 3633-3652. In some embodiments, the masking peptide inhibits or reduces the binding of the third domain (C) to the target antigen. In some embodiments, the cleavable linker comprises a cleavage site. In some embodiments, the cleavable site is recognized by a protease. In some embodiments, the protease is selected from the group consisting of a serine protease, a cysteine protease, an aspartate protease, a threonine protease, a glutamic acid protease, a metalloproteinase, a gelatinase, and an asparagine peptide lyase. In some embodiments, the protease is present in blood circulation. In some embodiments, the protease is selected from the group consisting of a Cathepsin, a Cathepsin B, a Cathepsin C, a Cathepsin D, a Cathepsin E, a Cathepsin H, a Cathepsin S, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26).

In some embodiments, the cleavable linker comprises an amino acid sequence having at least 80% homology to SEQ ID NOS: 3688-3770 and SEQ ID NO: 3878. In some embodiments, the cleavable linker comprises an amino acid sequence of SEQ ID NOS: 3688-3770 and SEQ ID NO: 3878. In some embodiments, the target antigen is a tumor antigen. In some embodiments, the third domain specifically binds to a target antigen selected from the group consisting of: CD19 (B-lymphocyte antigen CD19, B-Lymphocyte Surface Antigen B4, T-Cell Surface Antigen Leu-12, CVID3), PSMA (prostate specific membrane antigen), MSLN (mesothelin), BCMA (B-cell maturation antigen), DLL3 (Delta-like ligand 3), FLT3 (FMS-like tyrosine kinase 3), CD20 (B-lymphocyte antigen CD20, MS4A1, B1, Bp35, CVID5, LEU-16, MS4A2, S7, membrane spanning 4-domains A1), CD22 (SIGLEC-2, SIGLEC2), CD25 (IL2RA, interleukin-2 receptor alpha chain), CD27 (S152, S152. LPFS2, T14, TNFRSF7, Tp55), CD30 (TNFRSF8), CD33 (Siglec-3, sialic acid binding Ig-like lectin 3, SIGLEC3, SIGLEC-3, gp67, p67), CD37 (GP52-40, TSPAN26), CD38 (cyclic ADP ribose hydrolase, ADPRC1, ADPRC 1), CD40 (Bp50, CDW40, TNFRSF5, p50), CD44 (HCAM, homing cell adhesion molecule), Pgp-1 (phagocytic glycoprotein-1), Hermes antigen, lymphocyte homing receptor, ECM-III, and HUTCH-1), CD48 (BLAST-1, B-lymphocyte activation marker, SLAMF2, signaling lymphocytic activation molecule 2), CD52 (CAMPATH-1 antigen), CD70, CD73 (NT5E, ecto-5′-nucleotidase), CD39 (ENTPD1, Ectonucleoside triphosphate diphosphohydrolase-1), CD74 (HLA class II histocompatibility antigen gamma chain, HLA-DR antigens-associated invariant chain), CD79b (immunoglobulin-associated beta), CD80 (B7-1), CD86 (B7-2), CD123 (IL3RA, interleukin-3 receptor), CD133 (PROM1), CD137 (TNFRSF9, tumor necrosis factor receptor superfamily member 9, 4-1BB, ILA, induced by lymphocyte activation), CD138 (SDC1), alpha fetoprotein (AFP), c-Met; c-Kit; CD371 (CLEC12A, C-type lectin domain family 12 member A, CLL1)); CD370 (CLEC9A, C-type lectin domain containing 9A); cadherin 3 (CDH3, p-cadherin, PCAD); carbonic anhydrase 6 (CA6); carbonic anhydrase 9 (CA9, CA1X); carcinoembryonic antigen related cell adhesion molecule 3 (CEACAM3); carcinoembryonic antigen related cell adhesion molecule 5 (CEACAM5); CD66c (CEACAM6, carcinoembryonic antigen related cell adhesion molecule 6); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EPCAM); erb-b2 receptor tyrosine kinase 2 (ERBB2, HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRa); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A1 l (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); CD352 (SLAMF6, SLAM family member 6); CD319 (SLAMF7, SLAM family member 7, 19A, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6, LIV1); STEAP family member 1 (STEAP1); STEAP family member 2 (STEAP2); CD134 (TNFRSF4, TNF receptor superfamily member 4, OX40); CD137L (TNFSF9, TNF superfamily member 9, 4-1BB-L); CD261 (TNFRSF10A, TNF receptor superfamily member 10a, DR4, TRAILR1); CD262 (TNFRSF10B, TNF receptor superfamily member 10b, DR5, TRAILR2); CD267 (TNFRSF13B, TNF receptor superfamily member 13B, TACI, IGAD2); CD269 (TNFRSF17, TNF receptor superfamily member 17, BCMA); CD357 (TNFRSF18, TNF receptor superfamily member 18 GITR); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GM1; sialyl Lewis adhesion molecule (sLe); ROR1; CD30; and Lewis Y antigen.

In some embodiments, the third domain specifically binds to CD19. In some embodiments, the third domain comprises an amino acid sequence having at least 80% homology to SEQ ID NOS: 3771-3792. In some embodiments, the third domain comprises an amino acid sequence having at least 90% homology to SEQ ID NOS: 3771-3792. In some embodiments, the third domain comprises an amino acid sequence of SEQ ID NOS: 3771-3792. In some embodiments, the third domain comprises an amino acid sequence of SEQ ID NO: 3771. In some embodiments, the domains of the half-life extended immune cell engaging protein are linked in one of the following orders: H₂N-(C)-(B)-(A)-COOH, H₂N-(C)-(A)-(B)-COOH, or by linkers L1 and L2 in one of the following orders: H₂N-(C)-L1-(B)-L2-(A)-COOH, H₂N-(C)-L1-(A)-L2-(B)-COOH. In some embodiments, the half-life extended immune cell engaging protein comprises an amino acid sequence of SEQ ID NOS: 3839-3843. In some embodiments, the half-life extended immune cell engaging protein comprises an amino acid sequence of SEQ ID NOS: 3839-3843.

In some embodiments, the third domain specifically binds to CD20. In some embodiments, the third domain comprises an amino acid sequence having at least 80% homology to SEQ ID NOS: 3793-3808 and 3880. In some embodiments, the third domain comprises an amino acid sequence having at least 90% homology to SEQ ID NOS: 3793-3808 and 3880. In some embodiments, the third domain comprises an amino acid sequence of SEQ ID NOS: 3793-3808 and 3880. In some embodiments, the third domain comprises an amino acid sequence of SEQ ID NO: 3793. In some embodiments, the domains of the half-life extended immune cell engaging protein are linked in the following order: H₂N-(A)-(B)-(C)-COOH, or by linkers L1 and L2 in the following order: H₂N-(A)-L1-(B)-L2-(C)-COOH.

In some embodiments, the third domain specifically binds to CD33. In some embodiments, the third domain comprises an amino acid sequence having at least 80% homology to SEQ ID NOS: 3809-3823. In some embodiments, the third domain comprises an amino acid sequence having at least 90% homology to SEQ ID NOS: 3809-3823. In some embodiments, the third domain comprises an amino acid sequence of SEQ ID NOS: 3809-3823. In some embodiments, the third domain comprises an amino acid sequence of SEQ ID NO: 3809. In some embodiments, the domains of the half-life extended immune cell engaging protein are linked in one of the following orders: H₂N-(C)-(B)-(A)-COOH, H₂N-(A)-(B)-(C)-COOH, H₂N-(C)-(A)-(B)-COOH, H₂N-(A)-(C)-(B)-COOH, or by linkers L1 and L2 in one of the following orders: H₂N-(C)-L1-(B)-L2(A)-COOH, H₂N-(A)-L1-(B)-L2-(C)-COOH, H₂N-(C)-L1-(A)-L2-(B)-COOH, H₂N-(A)-L1-(C)-L2(B)-COOH. In some embodiments, the half-life extended immune cell engaging protein comprises an amino acid sequence of SEQ ID NOS: 3833-3837. In some embodiments, the half-life extended immune cell engaging protein comprises an amino acid sequence of SEQ ID NOS: 3834-3837.

In some embodiments, the third domain specifically binds to FLT3. In some embodiments, the third domain is a single domain antibody that specifically binds to FLT3. In some embodiments, the third domain comprises a CDR1 comprising an amino acid selected from the group consisting of SEQ ID NOS: 1080-1155 and 3497-3498, a CDR2 comprising an amino acid selected from the group consisting of SEQ ID NOS: 1156-1231, and 3499-3500, a CDR3 comprising an amino acid selected from the group consisting of SEQ ID NOS: 1232-1307, and 3501-3502. In some embodiments, the CDR1 comprises an amino acid sequence selected from the group consisting of SEQ ID NOS: 1150, 1152, 3497, and 3498; the CDR2 comprises an amino acid sequence selected from the group consisting of SEQ ID NOS: 1226, 1228, 3499, and 3500; the CDR3 comprises an amino acid sequence selected from the group consisting of SEQ ID NOS: 1302, 1304, 3501, and 3502. In some embodiments, the third domain comprises an amino acid sequence selected from the group consisting of SEQ ID NOS: 1004-1079 and 3495-3496. In some embodiments, the third domain comprises an amino acid sequence selected from the group consisting of: SEQ ID NOS: 1074, 1076, 3495, and 3496. In some embodiments, the third domain comprises an amino acid sequence of SEQ ID NO: 1074. In some embodiments, the domains of the half-life extended immune cell engaging protein are linked in one of the following orders: H₂N-(C)-(B)-(A)-COOH, H₂N-(A)-(B)-(C)-COOH, H₂N-(C)-(A)-(B)-COOH, H₂N-(A)-(C)-(B)-COOH, or by linkers L1 and L2 in one of the following orders: H₂N-(C)-L1-(B)-L2(A)-COOH, H₂N-(A)-L1-(B)-L2-(C)-COOH, H₂N-(C)-L1-(A)-L2-(B)-COOH, H₂N-(A)-L1-(C)-L2(B)-COOH. In some embodiments, the half-life extended immune cell engaging protein comprises an amino acid sequence of SEQ ID NOS: 3844-3849. In some embodiments, the half-life extended immune cell engaging protein comprises an amino acid sequence of SEQ ID NOS: 3846-3849. In some embodiments, the third domain comprises an amino acid sequence of SEQ ID NO: 1076. In some embodiments, the domains of the half-life extended immune cell engaging protein are linked in the following order: H₂N-(C)-(B)-(A)-COOH, or by linkers L1 and L2 in the following order: H₂N-(C)-L1-(B)-L2(A)-COOH. In some embodiments, the half-life extended immune cell engaging protein comprises an amino acid sequence of SEQ ID NOS: 3850-3855. In some embodiments, the half-life extended immune cell engaging protein comprises an amino acid sequence of SEQ ID NO: 3854.

In some embodiments, the third domain is a single domain antibody that specifically binds to PSMA. In some embodiments, the third domain comprises a CDR1 comprising an amino acid sequence selected from the group consisting of SEQ ID NOS: 462-465, a CDR2 comprising an amino acid sequence selected from the group consisting of SEQ ID NOS: 466-472, and a CDR3 comprising an amino acid sequence selected from the group consisting of SEQ ID NOS: 474-475. In some embodiments, the CDR1 comprises the amino acid sequence of SEQ ID NO: 462, the CDR2 comprises the amino acid of SEQ ID NO: 473, the CDR3 comprises the amino acid sequence of SEQ ID NO: 474. In some embodiments, the third domain comprises an amino acid sequence selected from the group consisting of SEQ ID NOS: 476-489. In some embodiments, the third domain comprises the amino acid sequence of SEQ ID NOS: 489. In some embodiments, the half-life extended immune cell engaging protein comprises an amino acid sequence of SEQ ID NO: 3824-3831.

In some embodiments, the third domain is a single domain antibody that specifically binds to MSLN. In some embodiments, the third domain comprises a CDR1 comprising an amino acid sequence selected from the group consisting of SEQ ID NOS: 490-528, a CDR2 comprising an amino acid selected from the group consisting of SEQ ID NOS: 529-567, and a CDR3 comprising an amino acid selected from the group consisting of SEQ ID NOS: 568-606. In some embodiments, the CDR1 comprises the amino acid sequence of SEQ ID NO: 523, the CDR2 comprises the amino acid of SEQ ID NO: 562, the CDR3 comprises the amino acid sequence of SEQ ID NO: 601. In some embodiments, the third domain comprises an amino acid sequence selected from the group consisting of SEQ ID NOS: 607-650. In some embodiments, the third domain comprises the amino acid sequence of SEQ ID NO: 647. In some embodiments, the half-life extended immune cell engaging protein comprises an amino acid sequence of SEQ ID NO: 3856-3858.

In some embodiments, the third domain is a single domain antibody that specifically binds to BCMA. In some embodiments, the third domain CDR1 comprises comprising an amino acid selected from the group consisting of SEQ ID NOS: 1-115, a CDR2 comprising an amino acid selected from the group consisting of SEQ ID NOS: 116-230, and a CDR3 comprising an amino acid selected from the group consisting of SEQ ID NOS: 231-345. In some embodiments, the CDR1 comprises the amino acid sequence of SEQ ID NO: 73, the CDR2 comprises the amino acid of SEQ ID NO: 188, the CDR3 comprises the amino acid sequence of SEQ ID NO: 303. In some embodiments, the third domain comprises an amino acid sequence selected from the group consisting of SEQ ID NOS: 346-461. In some embodiments, the third domain comprises the amino acid sequence of SEQ ID NO: 383.

In some embodiments, the third domain is a single domain antibody that specifically binds to DLL3. In some embodiments, the third domain comprises a CDR1 comprising an amino acid selected from the group consisting of SEQ ID NOS: 1751-2193, a CDR2 comprising an amino acid selected from the group consisting of SEQ ID NOS: 2194-2636, and a CDR3 comprising an amino acid selected from the group consisting of SEQ ID NOS: 2637-3080. In some embodiments, the CDR1 comprises the amino acid sequence of SEQ ID NO: 2182, the CDR2 comprises the amino acid of SEQ ID NO: 2625, the CDR3 comprises the amino acid sequence of SEQ ID NO: 3069. In some embodiments, the third domain comprises an amino acid sequence selected from the group consisting of SEQ ID NOS: 1308-1750. In some embodiments, the third domain comprises the amino acid sequence of SEQ ID NO: 1739.

In some embodiments, the third domain is a single domain antibody that specifically binds to EGFR. In some embodiments, the third domain comprises a CDR1 comprising an amino acid selected from the group consisting of SEQ ID NOS: 651-699, a CDR2 comprising an amino acid selected from the group consisting of SEQ ID NOS: 700-748, a CDR3 comprising an amino acid selected from the group consisting of SEQ ID NOS: 479-797. In some embodiments, the third domain comprises an amino acid sequence selected from the group consisting of SEQ ID NOS: 798-846.

In some embodiments, the third domain is a single domain antibody that specifically binds to EpCAM. In some embodiments, the third domain comprises a CDR1 comprising an amino acid selected from the group consisting of SEQ ID NOS: 847-884, a CDR2 comprising an amino acid selected from the group consisting of SEQ ID NOS: 885-922, a CDR3 comprising an amino acid selected from the group consisting of SEQ ID NOS: 923-960. In some embodiments, the CDR1 comprises the amino acid sequence of SEQ ID NO: 874 or 863, the CDR2 comprises the amino acid of SEQ ID NO: 885 or 901, the CDR3 comprises the amino acid sequence of SEQ ID NO: 923 or 939. In some embodiments, the third domain comprises a sequence selected from the group consisting of SEQ ID NOS: 961-1003. In some embodiments, the third domain comprises the amino acid sequence of SEQ ID NO: 999 or 1003.

In some embodiments, the first domain comprises a single-chain variable fragment (scFv) specific to human CD3. In some embodiments, the scFv specific to human CD3 comprises a variable heavy chain region (VH), a variable light chain region (VL), and a linker, wherein VH comprises complementarity determining regions HC CDR1, HC CDR2, and HC CDR3, and wherein VL comprises complementarity determining regions LC CDR1, LC CDR2, and LC CDR3. In some embodiments, the HC CDR1 comprises an amino acid sequence selected from the group consisting of SEQ ID NOS: 3081, and 3087-3098, the HC CDR2 comprises an amino acid sequence selected from the group consisting of SEQ ID NOS: 3082, and 3099-3109, the HC CDR3 comprises an amino acid sequence selected from the group consisting of SEQ ID NOS: 3083, and 3110-3119. In some embodiments, the HC CDR1 comprises the amino acid sequence of SEQ ID NO: 3097, the HC CDR2 comprises the amino acid sequence of SEQ ID NO: 3108, the HC CDR3 comprises the amino acid sequence of SEQ ID NO: 3110. In some embodiments, the LC CDR1 comprises an amino acid sequence selected from the group consisting of SEQ ID NOS: 3084, and 3120-3132, the LC CDR2 comprises an amino acid sequence selected from the group consisting of SEQ ID NOS: 3085, and 3099-3109, the LC CDR3 comprises an amino acid sequence selected from the group consisting of SEQ ID NOS: 3086, and 3146-3152. In some embodiments, the LC CDR1 comprises the amino acid sequence of SEQ ID NO: 3120, the LC CDR2 comprises the amino acid sequence of SEQ ID NO: 3145, the LC CDR3 comprises the amino acid sequence of SEQ ID NO: 3146. In some embodiments, the first domain comprises an amino acid sequence selected from the group consisting of SEQ ID NOS: 3153-3169. In some embodiments, the first domain comprises the amino acid sequence of SEQ ID NO: 3153. In some embodiments, the linker comprises an amino acid sequence GGGGSGGGGSGGGGS (SEQ ID NO: 3868).

In some embodiments, the second domain comprises a single domain antibody (sdAb) which specifically binds to HSA. In some embodiments, the sdAb which specifically binds to HSA comprises complementarity determining regions CDR1, CDR2, and CDR3, wherein the CDR1 comprises an amino acid sequence selected from the group consisting of SEQ ID NOS: 3170, and 3173-3175, the CDR2 comprises an amino acid sequence selected from the group consisting of SEQ ID NOS: 3171, and 3176-3181, the CDR3 comprises an amino acid sequence selected from the group consisting of SEQ ID NOS: 3172, and 8182-3183. In some embodiments, the CDR1 comprises an amino acid sequence of SEQ ID NO: 3174, the CDR2 comprises an amino acid of SEQ ID NO: 3178, the CDR3 comprises an amino acid sequence of SEQ ID NO: 3183. In some embodiments, the second domain comprises an amino acid sequence selected from the group consisting of SEQ ID NOS: 3184-3193. In some embodiments, the second domain comprises the amino acid sequence of SEQ ID NO: 3190. In some embodiments, linkers L1 and L2 are each, independently, (GS)ₙ (SEQ ID NO: 3859), (GGS)ₙ (SEQ ID NO: 3860), (GGGS)ₙ (SEQ ID NO: 3861), (GGSG)ₙ (SEQ ID NO: 3862), (GGSGG)ₙ (SEQ ID NO: 3863), (GGGGS)ₙ (SEQ ID NO: 3864), (GGGGG)ₙ (SEQ ID NO: 3865), or (GGG)ₙ (SEQ ID NO: 3866), wherein n is 1, 2, 3, 4, 5, 6, 7, 8, 9, or 10, (GGGGSGGGGSGGGGSGGGGS)(SEQ ID NO: 3867), (GGGGSGGGGSGGGGS) (SEQ ID NO: 3868), LPETG (SEQ ID NO: 3869), (GGGGSGGGS) (SEQ ID NO: 3871) or SGGG (SEQ ID NO: 3872). In some embodiments, linkers L1 and L2 are each, independently, GGGGSGGGS (SEQ ID NO: 3871).

Provided herein is method for the treatment or amelioration of a disease comprising administrating to a subject in need thereof a pharmaceutical composition according to the description herein. In some embodiments, the disease is a cancer. Provided herein is method for increasing survival in a subject suffering from a cancer, the method comprising administering to the subject a pharmaceutical composition according to the description herein. Provided herein is method of reducing tumor size, the method comprising administering to a subject from a cancer a pharmaceutical composition according to the description herein. In some embodiments, the cancer is selected from the group consisting of: mesothelioma, a prostate cancer, a breast cancer, a brain cancer, a bladder cancer, a pancreatic carcinoma, a renal cancer, a solid tumor, a liver cancer, a leiomyosarcoma, an endometrium cancer, a breast cancer, a female reproductive system cancer, an ovarian carcinoma, a soft tissue sarcoma, a gastric cancer, a digestive/gastrointestinal cancer, a colorectal cancer, a glioblastoma multiforme, a head and neck cancer, a squamous cell carcinoma, a colon cancer, a gastric cancer, a rhabdomyosarcoma, an adrenal cancer, a lung cancer, an esophageal cancer, a colon cancer, a lung cancer, a non-small cell lung carcinoma (NSCLC), a neuroblastoma, a melanoma, glioblastoma multiforme, an ovarian cancer, an endocrine cancer, a respiratory/thoracic cancer, an anal cancer, a gastro-esophageal cancer, a thyroid cancer, a cervical cancer, an endometrial cancer, a hematological cancer, a leukemia, a lymphocytic leukemia, a multiple myeloma, a lymphoma, a Hodgkin's lymphoma, a non-Hodgkin's lymphoma, a lymphocytic leukemia, an anaplastic large-cell lymphoma (ALCL), or a myeloid leukemia. In some embodiments, the cancer is the prostate cancer. In some embodiments, the cancer is the ovarian carcinoma. In some embodiments, the cancer is the pancreatic carcinoma. In some embodiments, the cancer is the mesothelioma. In some embodiments, the cancer is the lung cancer.

Provided herein is a pharmaceutical composition comprising an extended-release binding protein which comprises an antigen binding protein, a masking peptide, and a cleavable linker, wherein upon administering to a subject, the extended-release binding protein gradually releases an active version of the extended-release binding protein when the cleavable linker is cleaved in systemic circulation. In some embodiments, the antigen binding protein comprises an antigen binding domain that binds to a target antigen. In some embodiments, the masking peptide inhibits or reduces the binding of the antigen binding protein to the target antigen. In some embodiments, the antigen binding protein comprises an immune cell engaging domain. In some embodiments, the immune cell engaging domain comprises a T cell engaging domain. In some embodiments, the T cell engaging domain binds a CD3 molecule. In some embodiments, the masking peptide inhibits or reduces the binding of the antigen binding protein to the CD3 molecule. In some embodiments, the antigen binding protein comprises a half-life extension domain. In some embodiments, the half-life extension domain binds a human serum albumin (HSA). In some embodiments, the cleavable linker comprises a cleavage site. In some embodiments, the cleavable site is recognized by a protease. In some embodiments, wherein the masking peptide is covalently linked to the N-terminus or the C-terminus of the antigen binding protein through the cleavable linker.

Provided herein is a method of making an extended-release binding protein comprising adding a cleavable linker and a masking peptide to an antigen binding protein, wherein upon administering to a subject, the extended-release binding protein gradually releases an active version of the extended-release binding protein when the cleavable linker is cleaved in systemic circulation. In some embodiments, the antigen binding protein comprises an antigen binding domain that binds to a target antigen. In some embodiments, the masking peptide inhibits or reduces the binding of the antigen binding protein to the target antigen. In some embodiments, the antigen binding protein comprises an immune cell engaging domain. In some embodiments, the immune cell engaging domain comprises a T cell engaging domain. In some embodiments, the T cell engaging domain binds a CD3 molecule. In some embodiments, the masking peptide inhibits or reduces the binding of the antigen binding protein to the CD3 molecule. In some embodiments, the antigen binding protein comprises a half-life extension domain. In some embodiments, the half-life extension domain binds a human serum albumin (HSA). In some embodiments, the cleavable linker comprises a cleavage site. In some embodiments, the cleavable site is recognized by a protease. In some embodiments, the masking peptide is covalently linked to the N-terminus or the C-terminus of the antigen binding protein through the cleavable linker.

Provided herein is a method of increasing the therapeutic index of an antigen binding protein, comprising adding a cleavable linker and a masking peptide to the antigen binding protein to form an extended-release binding protein, wherein upon administering to a subject, the extended-release binding protein gradually releases an active version of the extended-release binding protein when the cleavable linker is cleaved in systemic circulation. Provided herein is method of decreasing the CRS level in a subject, comprising adding a cleavable linker and a masking peptide to an antigen binding protein to form an extended-release binding protein, wherein upon administering to the subject, the extended-release binding protein gradually releases an active version of the extended-release binding protein when the cleavable linker is cleaved in systemic circulation. Provided herein is method of gradually increase an active drug's concentration in a subject's systemic circulation comprising administering an extended-release binding protein to the subject, wherein the extended-release binding protein comprises an antigen binding protein, a masking peptide, and a cleavable linker, wherein the extended-release binding protein releases the active drug upon cleavage of the cleavable linker in systemic circulation.

Provided herein is a pharmaceutical composition comprising a protein which comprises a binding moiety, a masking peptide, and a cleavable linker, wherein the masking peptide is covalently linked to the binding moiety through the cleavable linker, and wherein the cleavable linker is significantly cleaved in systemic circulation.

Provided herein is a pharmaceutical composition comprising a protein which comprises a binding moiety, a masking peptide, and a cleavable linker, wherein the masking peptide is covalently linked to the binding moiety through the cleavable linker, and wherein a half-life of the protein in systemic circulation is longer than a comparable protein without the masking peptide.

Provided herein is a pharmaceutical composition comprising a protein which comprises a binding moiety, a masking peptide, and a cleavable linker, wherein the masking peptide is covalently linked to the binding moiety through the cleavable linker, and wherein a comparable protein without the masking peptide has a nonlinear pharmacokinetics (PK) across a dose range evaluated, and wherein the protein has an improved linearity of PK across the dose range evaluated compared to the comparable protein.

Provided herein is a pharmaceutical composition comprising a protein which comprises a binding moiety, a masking peptide, and a cleavable linker, wherein the masking peptide is covalently linked to the binding moiety through the cleavable linker, wherein the binding moiety specifically binds to a target in a subject, and wherein a molar amount of the protein's binding moiety bound to the target when administered to the subject is lower compared to a molar amount of a binding moiety of a comparable protein without the masking peptide when administered to the subject at a same dose level.

Provided herein is a pharmaceutical composition comprising a protein which comprises a binding moiety, a masking peptide, and a cleavable linker, wherein the masking peptide is covalently linked to the binding moiety through the cleavable linker, wherein the binding moiety specifically binds to a target in a subject, and wherein a first binding rate between the protein's binding moiety and the target when administered to the subject is lower compared to a second binding rate between a binding moiety of a comparable protein without the masking peptide and the target.

In some embodiments, the binding moiety specifically binds to ICOS (inducible T cell co-stimulator, CD278), OX40 (CD134, TNFRSF4, tumor necrosis factor receptor superfamily member 4), CD40 (TNFRSF5, tumor necrosis factor receptor superfamily member 5), DR5 (death receptor 5, TRAIL receptor 2), GITR (glucocorticoid-induced TNFR-related protein, TNFRSF18, tumor necrosis factor receptor superfamily member 18), 4-1BB (CD137, TNFRSF9, tumor necrosis factor receptor superfamily member 9) or any combinations thereof. In some embodiments, the protein comprises an antibody selected from the group consisting of GSK3359609, PF-8600, JNJ-64457107, CP-870,893, SGN-40 (Dacetuzumab), MEDI3039, ABBV-621, MEDI1873, AMG 228, PF-05082566 (Utomilumab), and urelumab.

Provided herein is a method of treating or ameliorating a disease comprising administrating to a subject in need thereof a pharmaceutical composition described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows an exemplary construct of an extended-release binding protein. FIG. 1B shows an exemplary construct of the active version of the extended-release binding protein. FIG. 1C shows the predicted concentrations of the intact version and the active version in systemic circulation after multiple administration events. FIG. 1D shows the predicted concentration of the active version of a corresponding antigen binding protein without the masking peptide in systemic circulation after multiple administration events.

FIGS. 2A-L illustrates additional extended-release binding protein constructs with various configurations of the anti-target, anti-albumin and anti-CD3 binding domains. FIG. 2A illustrates an intact version of an extended-release binding protein construct. FIG. 2B illustrate an intact version of an extended-release binding protein construct. FIG. 2C illustrate an intact version of an extended-release binding protein construct. FIG. 2G illustrate an intact version of an extended-release binding protein construct. FIG. 2H illustrate an intact version of an extended-release binding protein construct. FIG. 2I illustrate an intact version of an extended-release binding protein construct. FIG. 2D illustrates an active version after cleavage of the linker that contain linker amino acids left after protease cleavage (as described herein, these remaining amino acids are referred to as “stub”). FIG. 2E illustrates an active version after cleavage of the linker that contain stub. FIG. 2F illustrates an active version after cleavage of the linker that contain stub. FIG. 2J illustrates an active version after cleavage of the linker that contain stub. FIG. 2K illustrates an active version after cleavage of the linker that contain stub. FIG. 2L illustrates an active version after cleavage of the linker that contain stub.

FIG. 3 Analytical cation exchange chromatography demonstrates the purity of three PSMA binding proteins.

FIG. 4 demonstrates slow activation of peptide masked PSMA targeting extended-release binding proteins in TDCC assay measured in the presence of HSA.

FIG. 5 demonstrates the masking effects of various peptide masks engineered into PSMA binding proteins measured in the presence of HSA.

FIGS. 6A-B demonstrates the masking effects of various peptide masks engineered into MSLN binding proteins.

FIG. 7 demonstrates the TDCC assay results of various CD19 targeting proteins measured in the presence of HSA.

FIG. 8 demonstrates the TDCC assay results of various FLT3 targeting proteins measured in the presence of HSA.

FIG. 9 demonstrates the TDCC assay results of various FLT3 targeting protein measured in the presence of HSA.

FIG. 10 demonstrates the TDCC assay results of various CD33 targeting proteins measured in the presence of HSA.

FIG. 11 demonstrates the TDCC assay results of CD19 targeting proteins in the T:C:A or T:A:C configuration measured in the presence of HSA.

FIG. 12 demonstrates the TDCC assay results of FLT3 targeting proteins in the T:C:A configuration measured in the presence of HSA.

FIG. 13A illustrates the binding of TriTAC-XR non-cleavable prodrug, cleavable prodrug, and active drug to human CD3ε by ELISA. FIG. 13B illustrates the binding of TriTAC-XR non-cleavable prodrug, cleavable prodrug, and active drug to human T cells as measured by flow cytometry.

FIG. 14A illustrates the pharmacokinetics of FLT3 TriTAC-XR-L001 in cynomolgus monkeys after single i.v. doses of 300. FIG. 14B illustrates the pharmacokinetics of FLT3 TriTAC-XR-L001 in cynomolgus monkeys after single i.v. doses of 1000 μg/kg. FIG. 14C illustrates the pharmacokinetics of FLT3 TriTAC-XR-L085 after a single i.v. doses of 1000 μg/kg. Two assays were used to quantify the Intact, Active, and Total amount of TriTAC-XR. Plotted are mean values measured in plasma samples collected from two test subjects per dose group.

FIG. 15 illustrates the amount of soluble FLT3L present in serum samples collected from cynomolgus monkeys after single i.v. doses of FLT3 TriTAC-XR-L001 (SEQ ID NO: 3873) or TriTAC-XR-L085 (SEQ ID NO: 3874). Data are plotted each individual test subject in each dose group.

FIG. 16 illustrates the amount of FLT3 transcript present in RNA prepared from bone marrow collected from cynomolgus monkeys after single i.v. doses of FLT3 TriTAC-XR-L001 (SEQ ID NO: 3873) or TriTAC-XR-L085 (SEQ ID NO: 3874). Plotted are technical replicates. Data are plotted each individual test subject in each dose group.

FIG. 17 illustrates the pharmacokinetics of FLT3 TriTAC-XR-L001 (SEQ ID NO: 3873) or a constitutively active FLT3 TriTAC (SEQ ID NO: 3875) in non-human primates (NHP).

FIG. 18 illustrates the amount of FLT3 transcript present in RNA prepared from bone marrow collected from cynomolgus monkeys after single i.v. doses of 1000 μg/kg FLT3 TriTAC-XR-L001 (SEQ ID NO: 3873) or a constitutively active FLT3 TriTAC (SEQ ID NO: 3875). Plotted are technical replicates. Data are plotted each individual test subject in each dose group.

FIG. 19 illustrates the predicted pharmacokinetics of repeat dosing of TriTAC-XR or TriTAC in NHP.

FIG. 20 shows the empirical pharmacokinetics of repeat dosing of CD20 TriTAC-XR in cyno monkeys.

FIG. 21A illustrates the peak IL-2 level after i.v. dosing of FLT3 TriTAC-XR (FLT3 TriTAC-XR-L001, SEQ ID NO: 3873) at 300 and 1000 μg/kg or a constitutively active FLT3 TriTAC (SEQ ID NO: 3875) at 10, 100, and 1000 μg/kg. Plotted are mean values measured in serum samples collected from two test subjects per dose group. FIG. 21B illustrates the peak IL-6 level after i.v. dosing of FLT3 TriTAC-XR (FLT3 TriTAC-XR-L001, SEQ ID NO: 3873) at 300 and 1000 μg/kg or a constitutively active FLT3 TriTAC (SEQ ID NO: 3875) at 10, 100, and 1000 μg/kg. Plotted are mean values measured in serum samples collected from two test subjects per dose group.

FIG. 22 illustrates the amount of soluble FLT3L present in serum samples collected from cynomolgus monkeys after single i.v. doses of FLT3 TriTAC-XR-L001 (SEQ ID NO: 3873) at 300 and 1000 μg/kg, or a constitutively active FLT3 TriTAC (SEQ ID NO: 3875) at 10, 100 and 1000 μg/kg. Plotted are mean values measured in serum samples collected from two test subjects per dose group.

FIG. 23 illustrates the efficacy of an a FLT3 TriTAC-XR in a mouse tumor model.

FIG. 24 illustrates the pharmacokinetics of CD19 TriTAC-XR or a constitutively active CD19 TriTAC in NHP.

FIG. 25A illustrates the peak IL-2 level after i.v. dosing of CD19 TriTAC-XR or a constitutively active CD19 TriTAC. FIG. 25B illustrates the peak IL-6 level after i.v. dosing of CD19 TriTAC-XR or a constitutively active CD19 TriTAC.

FIG. 26 illustrates that target cell depletion is comparable between CD19 TriTAC and CD19 TriTAC-XR.

FIG. 27 illustrates the pharmacokinetics of CD20 TriTAC-XR or a constitutively active CD20 TriTAC in NHP.

FIG. 28A illustrates the peak IL-2 level after i.v. dosing of CD20 TriTAC-XR or a constitutively active CD20 TriTAC. FIG. 28B illustrates the peak IL-6 level after i.v. dosing of CD20 TriTAC-XR or a constitutively active CD20 TriTAC.

FIG. 29 illustrates that target cell depletion is comparable between CD20 TriTAC and CD20 TriTAC-XR.

FIG. 30 illustrates exemplary constructs of a protein with masking peptide as described herein.

### DETAILED DESCRIPTION OF THE INVENTION

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

An “antibody” typically refers to a Y-shaped tetrameric protein comprising two heavy (H) and two light (L) polypeptide chains held together by covalent disulfide bonds and non-covalent interactions. Human light chains comprise a variable domain (VL) and a constant domain (CL) wherein the constant domain may be readily classified as kappa or lambda based on amino acid sequence and gene loci. Each heavy chain comprises one variable domain (VH) and a constant region, which in the case of IgG, IgA, and IgD, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (generally from about 10 to about 60 amino acids in IgG). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues. There are two types of native disulfide bridges or bonds in immunoglobulin molecules: interchain and intrachain disulfide bonds. The location and number of interchain disulfide bonds vary according to the immunoglobulin class and species. Interchain disulfide bonds are located on the surface of the immunoglobulin, are accessible to solvent and are usually relatively easily reduced. In the human IgG1 isotype there are four interchain disulfide bonds, one from each heavy chain to the light chain and two between the heavy chains. The interchain disulfide bonds are not required for chain association. As is well known the cysteine rich IgG1 hinge region of the heavy chain has generally been held to consist of three parts: an upper hinge, a core hinge, and a lower hinge. Those skilled in the art will appreciate that that the IgG1 hinge region contain the cysteines in the heavy chain that comprise the interchain disulfide bonds (two heavy/heavy, two heavy/light), which provide structural flexibility that facilitates Fab movements. The interchain disulfide bond between the light and heavy chain of IgG1 are formed between C214 of the kappa or lambda light chain and C220 in the upper hinge region of the heavy chain. The interchain disulfide bonds between the heavy chains are at positions C226 and C229 (all numbered per the EU index according to Kabat, et al., infra.)

As used herein the term “antibody” includes polyclonal antibodies, multiclonal antibodies, monoclonal antibodies, chimeric antibodies, deimmunized, humanized and primatized antibodies, CDR grafted antibodies, human antibodies, recombinantly produced antibodies, intrabodies, multispecific antibodies, bispecific antibodies, monovalent antibodies (e.g., a monovalent IgG), multivalent antibodies, anti-idiotypic antibodies, synthetic antibodies, including muteins and variants thereof, immunospecific antibody fragments such as: hcIgG, a V-NAR, Fv, Fd, Fab, F(ab′)2, F(ab′), Fab2, Fab3 fragments, single-chain fragments (e.g., di-scFv, scFv, scFvFc, scFv-zipper, scFab), disulfide-linked Fvs (sdFv), a Fd fragment consisting of the VH and CH1 domains, linear antibodies, single domain antibodies such as nanobodies or single variable domain antibodies comprising merely one variable domain such as sdAb (VH, VL, or VHH domains), “r IgG” (“half antibody”), diabodies, single chain diabodies, tandem diabodies (Tandab's), tandem di-scFv, tandem tri-scFv, “minibodies” are in some instances exemplified by a structure which is as follows: (VH-VL-CH3)2, (scFv-CH3)2, ((scFv)2-CH3+CH3), ((scFv)2-CH3) or (scFv-CH3-scFv)2, multibodies such as triabodies or tetrabodies; and derivatives thereof including Fc fusions and other modifications, and any other immunoreactive molecule so long as it comprises a domain having a binding site for preferential association or binding with an FLT3protein. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter alpha, delta, epsilon, gamma, and mu, respectively. Light chains of the antibodies from any vertebrate species can be assigned to one of two clearly distinct types, called kappa (kappa) and lambda (lambda), based on the amino acid sequences of their constant domains.

As used herein, “Variable region” or “variable domain” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a n-sheet configuration, connected by three CDRs, which form loops connecting, and in some cases forming part of, the βsheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. The assignment of amino acids to each domain, framework region and CDR is, in some embodiments, in accordance with one of the numbering schemes provided by Kabat et al. (1991) Sequences of Proteins of Immunological Interest (5th Ed.), US Dept. of Health and Human Services, PHS, NIH, NIH Publication NO: 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3rd Ed., Wily-VCH Verlag GmbH and Co or AbM (Oxford Molecular/MSI Pharmacopeia) unless otherwise noted.

“Variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. It is not intended that CDRs of the present disclosure necessarily correspond to the Kabat numbering convention.

The term “Framework” or “FR” residues (or regions) refer to variable domain residues other than the CDR or hypervariable region residues as herein defined. A “human consensus framework” is a framework which represents the most commonly occurring amino acid residue in a selection of human immunoglobulin VL or VH framework sequences.

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer softwares such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Alignment for purposes of determining percent amino acid sequence identity can for example be achieved using publicly available sequence comparison computer program ALIGN-2. The source code for the ALIGN-2 sequence comparison computer program is available with user documentation in the U.S. Copyright Office, Washington D.C., 20559, where it is registered under U.S. Copyright Registration NO: TXU510087. The ALIGN-2 program can be compiled for use on a UNIX operating system, such as a digital UNIX V4.0D. All sequence comparison parameters are set by the ALIGN-2 program and do not vary.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time-1 and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e., after drug absorption and distribution are complete). The half-time for drug elimination can be accurately determined from such a graph.

As used herein, the term “binding affinity” refers to the affinity of the proteins described in the disclosure to their binding targets, and is expressed numerically using “Kd” values. If two or more proteins are indicated to have comparable binding affinities towards their binding targets, then the Kd values for binding of the respective proteins towards their binding targets, are within ±2-fold of each other. If two or more proteins are indicated to have comparable binding affinities towards single binding target, then the Kd values for binding of the respective proteins towards said single binding target, are within ±2-fold of each other. If a protein is indicated to bind two or more targets with comparable binding affinities, then the Kd values for binding of said protein to the two or more targets are within ±2-fold of each other. In general, a higher Kd value corresponds to a weaker binding. In some embodiments, the “Kd” is measured by a radiolabeled antigen binding assay (RIA) or surface plasmon resonance assays using a BIAcore™-2000 or a BIAcore™-3000 (BIAcore, Inc., Piscataway, N.J.). In certain embodiments, an “on-rate” or “rate of association” or “association rate” or “kon” and an “off-rate” or “rate of dissociation” or “dissociation rate” or “koff” are also determined with the surface plasmon resonance technique using a BIAcore™-2000 or a BIAcore™-3000 (BIAcore, Inc., Piscataway, N.J.). In additional embodiments, the “Kd”, “kon”, and “koff” are measured using the OCTET® Systems (Pall Life Sciences). In an exemplary method for measuring binding affinity using the OCTET® Systems, the ligand, e.g. biotinylated human or cynomolgus FLT3 in case of an immune cell engaging protein comprising a FLT3 binding single domain antibody, is immobilized on the OCTET® streptavidin capillary sensor tip surface which streptavidin tips are then activated according to manufacturer's instructions using about 20-50 μg/ml human or cynomolgus FLT3 protein. A solution of PBS/Casein is also introduced as a blocking agent. For association kinetic measurements, FLT3 binding protein variants are introduced at a concentration ranging from about 10 ng/mL to about 100 μg/mL, about 50 ng/mL to about 5 μg/mL, or about 2 ng/mL to about 20 μg/mL. In some embodiments, the FLT3 binding single domain proteins are used at a concentration ranging from about 2 ng/mL to about 20 μg/mL. Complete dissociation is observed in case of the negative control, assay buffer without the binding proteins. The kinetic parameters of the binding reactions are then determined using an appropriate tool, e.g., ForteBio software.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

Generally, it should be noted that the term single domain antibody as used herein in its broadest sense is not limited to a specific biological source or to a specific method of preparation. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. For example, in some embodiments, the single domain antibodies of the disclosure are obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a species of mammal, such as from a human being, or by expression of a nucleic acid encoding such a camelized VH domain; (5) by “camelization” of a “domain antibody” or “Dab,” or by expression of a nucleic acid encoding such a camelized VH domain; (6) by using synthetic or semi-synthetic techniques for preparing proteins, polypeptides or other amino acid sequences; (7) by preparing a nucleic acid encoding a single domain antibody using techniques for nucleic acid synthesis known in the field, followed by expression of the nucleic acid thus obtained; and/or (8) by any combination of one or more of the foregoing.

In some embodiments, an immune cell engaging protein as described herein comprises an antibody, e.g., a single domain antibody targeting an antigen, e.g., PSMA, MSLN, BCMA, DLL3, EGFR, EpCAM, FLT3, comprising an amino acid sequence that corresponds to the amino acid sequence of a naturally occurring VHH domain, but that has been “humanized”, e.g., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above). This can be performed in a manner known in the field, which will be clear to the skilled person, for example on the basis of the further description herein. Again, it should be noted that such humanized single domain antibodies of the disclosure are obtained in any suitable manner known per se (e.g., as indicated under points (1)-(8) above) and thus are not strictly limited to polypeptides that have been obtained using a polypeptide that comprises a naturally occurring VHH domain as a starting material. In some additional embodiments, a single domain antibody, as described herein, comprises a single domain antibody with an amino acid sequence that corresponds to the amino acid sequence of a naturally occurring VH domain, but that has been “camelized”, i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized anti-MSLN single domain antibodies of the disclosure, in certain embodiments, are obtained in any suitable manner known in the field (i.e., as indicated under points (1)-(8) above) and thus are not strictly limited to polypeptides that have been obtained using a polypeptide that comprises a naturally occurring VH domain as a starting material. For example, as further described herein, both “humanization” and “camelization” is performed by providing a nucleotide sequence that encodes a naturally occurring VHH domain or VH domain, respectively, and then changing, one or more codons in said nucleotide sequence in such a way that the new nucleotide sequence encodes a “humanized” or “camelized” single domain antibody, respectively. This nucleic acid can then be expressed, so as to provide a desired single domain antibody of the disclosure. Alternatively, in other embodiments, based on the amino acid sequence of a naturally occurring VHH domain or VH domain, respectively, the amino acid sequence of the desired humanized or camelized single domain antibody of the disclosure, respectively, are designed and then synthesized de novo using known techniques for peptide synthesis. In some embodiments, based on the amino acid sequence or nucleotide sequence of a naturally occurring VHH domain or VH domain, respectively, a nucleotide sequence encoding the desired humanized or camelized single domain antibody of the disclosure, respectively, is designed and then synthesized de novo using known techniques for nucleic acid synthesis, after which the nucleic acid thus obtained is expressed in using known expression techniques, so as to provide the desired single domain antibody of the disclosure.

The terms “patient” or “subject” refers to any single subject for which therapy is desired or that is participating in a clinical trial, epidemiological study or used as a control, including humans and mammalian veterinary patients such as cattle, horses, dogs, and cats.

The term “therapeutic index” is the ratio of the TD₅₀ (or LD₅₀) to the ED₅₀, determined from quantal dose-response curves. ED₅₀ is the efficacious dose of a drug in 50% of subjects. TD₅₀ is the toxic dose of a drug in 50% of subjects for humans. LD₅₀ is the lethal dose of a drug for 50% of the population for animals. Therapeutic index demonstrates a quantitative measurement of the relative safety of the drug. A higher therapeutic index is preferable to a lower one: a patient would have to take a much higher dose of such a drug to reach the toxic threshold than the dose taken to elicit the therapeutic effect.

The term “significantly cleaved” means that a molecule is cleaved for more than 25%, 30%, 35%, 40%, 45%, 50%, 55%, 60%, 65%, 70%, 75%, 80%, 85%, 90%, 95% or 99%.

### Extended-Release Binding Protein

Described herein is a pharmaceutical composition comprising an extended-release binding protein which comprises an antigen binding protein/immune cell engaging protein, a masking peptide, and a cleavable linker, wherein the extended-release binding protein releases an active version of the extended-release binding protein after the cleavable linker is cleaved.

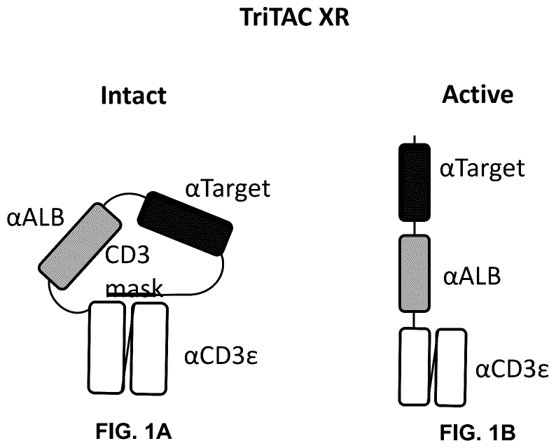

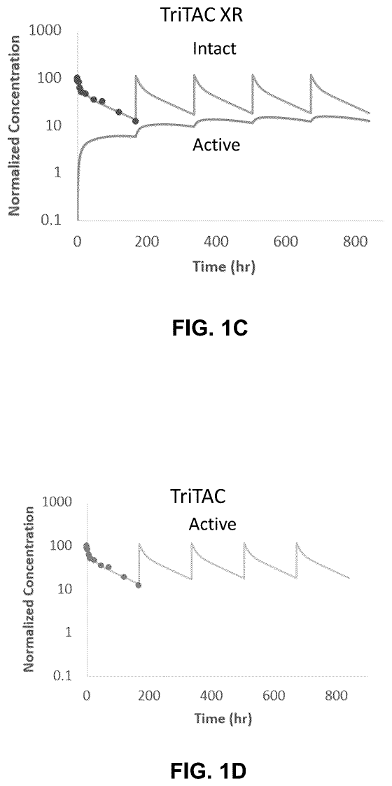

Without being bound by any theory, it is contemplated that the masking peptide, connected to the antigen binding protein/immune cell engaging protein via a cleavable linker, confers extended-release properties to the antigen binding protein/immune cell engaging protein. The extended-release binding protein is converted to an active form upon gradual cleaving of the cleavable linker, which removes the masking effect of the masking peptide, and releases the active form. The gradual release of the active form from the masked form (intact form) allows maintenance of a steady concentration of the active form for a longer duration of time, compared to directly administering the non-masked form. The extended-release binding protein is contemplated to have a higher therapeutic index than a corresponding antigen binding protein without the masking peptide. FIG. 1C shows the predicted concentrations of the intact version and the active version in systemic circulation after multiple administration events. FIG. 1D shows the predicted concentration of the active version of a corresponding antigen binding protein without the masking peptide in systemic circulation after multiple administration events. The concentration of the extended-release binding protein's active version is relatively constant compared to the concentration of the active version for a corresponding antigen binding protein without the masking peptide.

Cytokine release syndrome (CRS) is the major dose limiting toxicity, it is hypothesized that there are two factors contributing to CRS: 1) instant exposure to a high concentration of the active drug, and 2) exposures of the active drug that greatly exceed Cmin. The extended-release concept addresses both issues by having a slow initial release of active drug and by minimizing the difference between Cmax and Cmin. It is expected that relatively constant concentrations of the active drug in the serum result in relatively constant drug exposures to the tissues. Therefore, administration of the extended-release binding protein results in a lower Cytokine release syndrome (CRS) level than the CRS observed when a corresponding antigen binding protein without the masking peptide is administered.

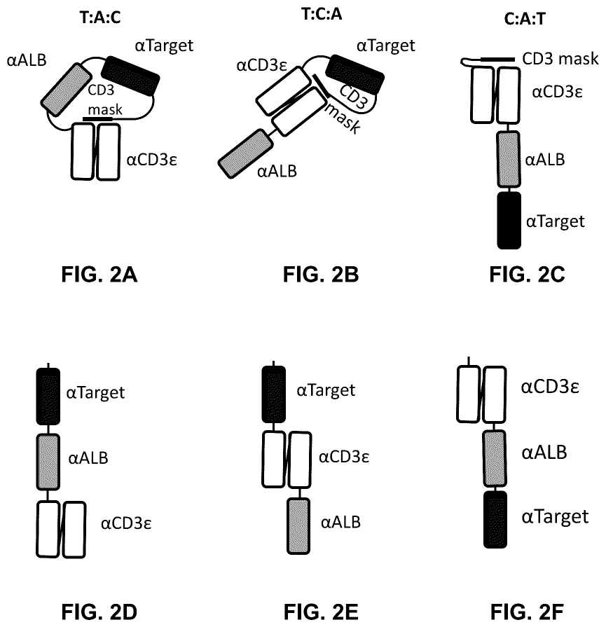

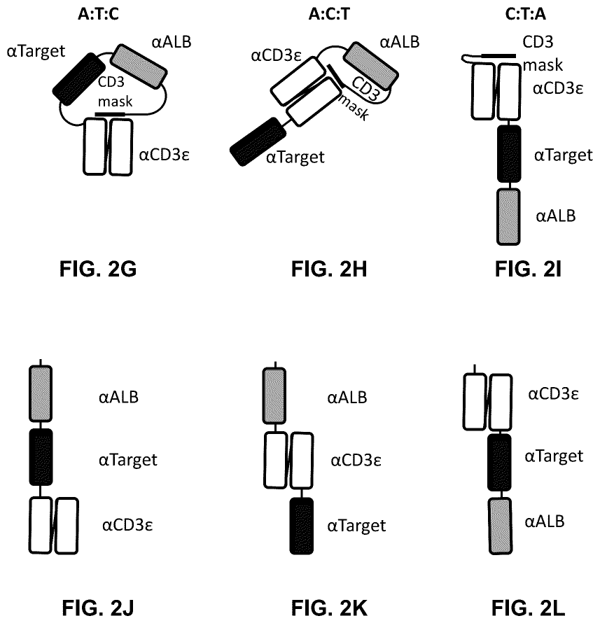

In some embodiments, the masking peptide is linked to the N-terminus or the C-terminus of the antigen binding protein/immune cell engaging protein, through a cleavable linker. Various exemplary configurations are provided in FIGS. 2A through 2L. In some embodiments, the masking peptide is linked to the N-terminus of the domain that specifically binds to HSA, wherein the domain that specifically binds to HSA is connected, via its C-terminus (e.g., through a linker) to the target antigen binding domain or the T-cell engaging domain. In some embodiments, the masking peptide is linked to the N-terminus of the target antigen binding domain, wherein the target antigen binding domain is connected, via its C-terminus (e.g., through a linker) to the domain that specifically binds to HSA or the T-cell engaging domain. In some embodiments, the masking peptide is linked to the C-terminus of the target antigen binding domain, wherein the target antigen binding domain is connected, via its N-terminus (e.g., through a linker) to the domain that specifically binds to HSA or the T-cell engaging domain. In some embodiments, the masking peptide is linked to the N-terminus of the T-cell engaging domain. In some embodiments, the masking peptide is linked to the C-terminus of the T-cell engaging domain. In some embodiments, the masking peptide is linked to the VH or VL domain of a T-cell engaging domain that is a single chain variable fragment (e.g., an scFv that is specific for a human CD3, such as CD3E). Alternative formats include, but are not limited to an extended-release binding protein comprising, from N-terminus to C-terminus:
a masking peptide-a cleavable linker-a target antigen binding domain-a serum albumin binding domain-a T-cell engaging domain;
a masking peptide-a cleavable linker-a target antigen binding domain-a T-cell engaging domain-a serum albumin binding domain;
a masking peptide-a cleavable linker-a T-cell engaging domain-a serum albumin binding domain-a target antigen binding domain;
a masking peptide-a cleavable linker-a T-cell engaging domain-a target antigen binding domain-a serum albumin binding domain;
a masking peptide-a cleavable linker-a serum albumin binding domain-a T-cell engaging domain-a target antigen binding domain;
a masking peptide-a cleavable linker-a serum albumin binding domain-a target antigen binding domain-a T-cell engaging domain;
a target antigen binding domain-a serum albumin binding domain-a T-cell engaging domain-a cleavable linker-a masking peptide;
a target antigen binding domain-a T-cell engaging domain-a serum albumin binding domain-a cleavable linker-a masking peptide;
a T-cell engaging domain-a serum albumin binding domain-a target antigen binding domain-a cleavable linker-a masking peptide;
a T-cell engaging domain-a target antigen binding domain-a serum albumin binding domain-a cleavable linker-a masking peptide;
a serum albumin binding domain-a T-cell engaging domain-a target antigen binding domain-a cleavable linker-a masking peptide;
a serum albumin binding domain-a target antigen binding domain-a T-cell engaging domain-a cleavable linker-a masking peptide.

The antigen binding protein/extended-release immune cell engaging protein of this disclosure, in some embodiments, comprises an amino acid sequence that is at least about 60% to about 100% identical to the sequence of SEQ ID NO: 3600, 3824-3858 or 3882.

### Immune Cell Engaging Proteins

Described herein, in some embodiments, is a pharmaceutical composition comprising an extended-release binding protein which comprises an immune cell engaging protein, a masking peptide, and a cleavable linker. In some embodiments, the immune cell engaging protein is a half-life extended protein. In some embodiments, the masking peptide is covalently linked to the N-terminus or the C-terminus of the half-life extended immune cell engaging protein through the cleavable linker. In some embodiments, the cleavable linker is significantly cleaved in systemic circulation. In some embodiments, the immune cell engaging protein is a bi-specific protein, a tri-specific protein or a multi-specific protein. In some embodiments, the immune cell engaging protein comprises a domain that binds to a bulk serum protein, e.g., a serum albumin. In some embodiments, the serum albumin is human serum albumin (HSA).

In some embodiments, the extended-release binding protein has a higher therapeutic index than a corresponding half-life-extended immune cell engaging protein without the masking peptide. In some embodiments, the administration of the extended-release binding protein results in a lower Cmax/Cmin ratio of an active version of the extended-release binding protein in systemic circulation than the Cmax/Cmin ratio when a corresponding half-life extended immune cell engaging protein without the masking peptide is administered. In some embodiments, multiple administration of the extended-release binding protein results in a more gradual increase of the level of an active version of the extended-release binding protein in systemic circulation than when a corresponding half-life-extended immune cell engaging protein without the masking peptide is administered. In some embodiments, administration of the extended-release binding protein results in a lower Cytokine release syndrome (CRS) level than the CRS observed when a corresponding half-life extended immune cell engaging protein without the masking peptide is administered.

In some embodiments, the immune cell engaging protein comprises an immune cell engaging domain. In some embodiments “an immune cell engaging domain” as used herein refers to one or more binding specificities that bind and/or activate an immune cell, e.g., a cell involved in an immune response. In some embodiments, the immune cell is selected from a natural killer (NK) cell, a B cell, a dendritic cell, a macrophage cell. The immune cell engaging domain, in some embodiments, is an antibody or an antigen binding fragment thereof, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to an immune cell antigen (e.g., the NK cell antigen, the B cell antigen, the dendritic cell antigen, and/or the macrophage cell antigen). In some embodiments, the immune cell engaging domain specifically binds to a target immune cell, e.g., binds preferentially to the target immune cell. For example, in some embodiments the immune cell engaging domain is an antibody or an antigen binding fragment thereof that binds to the immune cell antigen (e.g., the NK cell antigen, the B cell antigen, the dendritic cell antigen, and/or the macrophage cell antigen) with a dissociation constant of less than about 10 nM.

In some embodiments, the immune cell engaging domain comprises an NK cell engaging domain, a T cell engaging domain, a B cell engaging domain, a dendritic cell engaging domain, a macrophage cell engaging domain, or a combination thereof. In some embodiments, the immune cell engaging domain comprises the T-cell engaging domain. In some embodiments, the T cell engaging domain binds a CD3 molecule. In some embodiments, the CD3 molecule is at least one of: a CD3γ molecule, a CD3δ molecule, or a CD3ε molecule.

In some embodiments, the immune cell engaging protein comprises an antigen binding domain. In some examples, the antigen binding domain includes antibodies, single chain antibodies, Fabs, Fv, T-cell receptor binding domains, ligand binding domains, receptor binding domains, domain antibodies, single domain antibodies, minibodies, nanobodies, peptibodies, or various other antibody mimics (such as affimers, affitins, alphabodies, atrimers, CTLA4-based molecules, adnectins, anticalins, Kunitz domain-based proteins, avimers, knottins, fynomers, darpins, affibodies, affilins, monobodies and armadillo repeat protein-based proteins).

In some embodiments, the antigen is a tumor antigen, In some embodiments, the antigen is selected from the group consisting of: CD19 (B-lymphocyte antigen CD19, B-Lymphocyte Surface Antigen B4, T-Cell Surface Antigen Leu-12, CVID3), PSMA (prostate specific membrane antigen), MSLN (mesothelin), BCMA (B-cell maturation antigen), DLL3 (Delta-like ligand 3), EGFR (epidermal growth factor receptor), FLT3 (FMS-like tyrosine kinase 3), CD20 (B-lymphocyte antigen CD20, MS4A1, B1, Bp35, CVID5, LEU-16, MS4A2, S7, membrane spanning 4-domains A1), CD22 (SIGLEC-2, SIGLEC2), CD25 (IL2RA, interleulin-2 receptor alpha chain), CD27 (S152, S152. LPFS2, T14, TNFRSF7, Tp55), CD30 (TNFRSF8), CD33 (Siglec-3, sialic acid binding Ig-like lectin 3, SIGLEC3, SIGLEC-3, gp67, p67), CD37 (GP52-40, TSPAN26), CD38 (cyclic ADP ribose hydrolase, ADPRC1, ADPRC 1), CD40 (Bp50, CDW40, TNFRSF5, p50), CD44 (HCAM, homing cell adhesion molecule), Pgp-1 (phagocytic glycoprotein-1), Hermes antigen, lymphocyte homing receptor, ECM-III, and HUTCH-1), CD48 (BLAST-1, B-lymphocyte activation marker, SLAMF2, signaling lymphocytic activation molecule 2), CD52 (CAMPATH-1 antigen), CD70, CD73 (NT5E, ecto-5′-nucleotidase), CD39 (ENTPD1, Ectonucleoside triphosphate diphosphohydrolase-1), CD74 (HLA class II histocompatibility antigen gamma chain, HLA-DR antigens-associated invariant chain), CD79b (immunoglobulin-associated beta), CD80 (B7-1), CD86 (B7-2), CD123 (IL3RA, interleukin-3 receptor), CD133 (PROM1), CD137 (TNFRSF9, tumor necrosis factor receptor superfamily member 9, 4-1BB, ILA, induced by lymphocyte activation), CD138 (SDC1), alpha fetoprotein (AFP), c-Met; c-Kit; CD371 (CLEC12A, C-type lectin domain family 12 member A, CLL1)); CD370 (CLEC9A, C-type lectin domain containing 9A); cadherin 3 (CDH3, p-cadherin, PCAD); carbonic anhydrase 6 (CA6); carbonic anhydrase 9 (CA9, CA1X); carcinoembryonic antigen related cell adhesion molecule 3 (CEACAM3); carcinoembryonic antigen related cell adhesion molecule 5 (CEACAM5); CD66c (CEACAM6, carcinoembryonic antigen related cell adhesion molecule 6); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EpCAM); erb-b2 receptor tyrosine kinase 2 (ERBB2, HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRa); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); CD352 (SLAMF6, SLAM family member 6); CD319 (SLAMF7, SLAM family member 7, 19A, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6, LIV1); STEAP family member 1 (STEAP1); STEAP family member 2 (STEAP2); CD134 (TNFRSF4, TNF receptor superfamily member 4, OX40); CD137L (TNFSF9, TNF superfamily member 9, 4-1BB-L); CD261 (TNFRSF10A, TNF receptor superfamily member 10a, DR4, TRAILR1); CD262 (TNFRSF10B, TNF receptor superfamily member 10b, DR5, TRAILR2); CD267 (TNFRSF13B, TNF receptor superfamily member 13B, TACI, IGAD2); CD269 (TNFRSF17, TNF receptor superfamily member 17, BCMA); CD357 (TNFRSF18, TNF receptor superfamily member 18 GITR); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GM1; sialyl Lewis adhesion molecule (sLe); ROR1, CD30, and Lewis Y antigen. In some embodiments, the immune cell engaging protein specifically binds to PSMA, MSLN, BCMA, DLL3, EGFR, FLT3, or EpCAM.

Described herein, in some embodiments, is an immune cell engaging protein that comprises a first domain (A), which is a T cell engaging domain and specifically binds to human CD3, a second domain (B) which specifically binds to human serum albumin (HSA), and a third domain (C) which specifically binds to a target antigen. In some embodiments, the immune cell engaging protein's domains are linked in one of the following orders: H₂N-(C)-(B)-(A)-COOH, H₂N-(A)-(B)-(C)-COOH, H₂N-(B)-(A)-(C)-COOH, H₂N-(C)-(A)-(B)-COOH, H₂N-(A)-(C)-(B)-COOH, H₂N-(B)-(C)-(A)-COOH, or by linkers L1 and L2 in one of the following orders: H₂N-(C)-L1-(B)-L2(A)-COOH, H₂N-(A)-L1-(B)-L2-(C)-COOH, H₂N-(B)-L1-(A)-L2-(C)-COOH, H₂N-(C)-L1-(A)-L2-(B)-COOH, H₂N-(A)-L1-(C)-L2(B)-COOH, H₂N-(B)-L1-(C)-L2-(A)-COOH. In some embodiments, the linkers L1 and L2 are each independently selected from an amino acid sequence of SEQ ID NOS: 3190-3200.

The design of the immune cell engaging proteins described herein allows the binding domain to a target antigen, the third domain (C) described above, to be flexible in that the binding domain to a target antigen can be any type of binding domain, including but not limited to, domains from a monoclonal antibody, a polyclonal antibody, a recombinant antibody, a human antibody, or a humanized antibody. In some embodiments, the binding domain to a target antigen is a single chain variable fragments (scFv), single-domain antibody such as a heavy chain variable domain (VH), a light chain variable domain (VL), a variable domain (VHH) of a llama derived sdAb, or a variable domain (VHH) of camelid derived single domain antibody. In other embodiments, the binding domain to a target antigen is a non-Ig binding domain, e.g., antibody mimetic, such as anticalins, affilins, affibody molecules, affimers, affitins, alphabodies, avimers, DARPins, fynomers, kunitz domain peptides, and monobodies. In further embodiments, the binding domain to a target antigen is a ligand or peptide that binds to or associates with the target antigen. In yet further embodiments, the binding domain to a target antigen is a knottin. In yet further embodiments, the binding domain to a target antigen is a small molecular entity.

In some embodiments, the target antigen binding domain is an antibody or an antibody variant. As used herein, the term “antibody variant” refers to variants and derivatives of an antibody described herein. In certain embodiments, amino acid sequence variants of the antibodies described herein are contemplated. For example, in certain embodiments amino acid sequence variants of antibodies described herein are contemplated to improve the binding affinity and/or other biological properties of the antibodies. Exemplary methods for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant antibody, one or more hypervariable region residues of a parent antibody are substituted. In general, variants are then selected based on improvements in desired properties compared to a parent antibody, for example, increased affinity, reduced affinity, reduced immunogenicity, or increased pH dependence of binding.

In one embodiment, a single domain antibody corresponds to the VHH domains of naturally occurring heavy chain antibodies directed against a target antigen. As further described herein, such VHH sequences can generally be generated or obtained by suitably immunizing a species of Llama with the target antigen, (i.e., so as to raise an immune response and/or heavy chain antibodies directed against the target antigen), by obtaining a suitable biological sample from said Llama (such as a blood sample, serum sample or sample of B-cells), and by generating VHH sequences directed against the target antigen, starting from said sample, using any suitable technique known in the field.

In another embodiment, such naturally occurring VHH domains against the target antigen, are obtained from naïve libraries of Camelid VHH sequences, for example by screening such a library using the target antigen, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against a target antigen, involves suitably immunizing a transgenic mammal that is capable of expressing heavy chain antibodies (i.e., so as to raise an immune response and/or heavy chain antibodies directed against the target antigen), obtaining a suitable biological sample from said transgenic mammal (such as a blood sample, serum sample or sample of B-cells), and then generating VHH sequences directed against the target antigen, starting from said sample, using any suitable technique known in the field. For example, for this purpose, the heavy chain antibody-expressing rats or mice and the further methods and techniques described in WO 02/085945 and in WO 04/049794 can be used.

In some embodiments, a single domain antibody of the target antigen specific immune cell engaging protein comprises a single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining a target antigen binding single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide a target antigen binding single domain antibody of the disclosure or a nucleotide sequence or nucleic acid encoding the same.

In some embodiments, the target antigen binding domain is an antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, examples of the target antigen binding domain include but are not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the target antigen binding domain is a single domain antibody. In some embodiments, the single domain antibody comprises heavy chain variable complementarity determining regions (CDR), CDR1, CDR2, and CDR3.

In some embodiments, the immune cell engaging protein comprising the target antigen binding domain specifically binds to the target with equivalent or better affinity compared to that of an immune cell engaging protein comprising a reference target antigen binding domain, and the target antigen binding domain in such embodiments comprises an affinity matured target antigen binding domain, and is derived from a parental molecule, comprising one or more amino acid mutations (e.g., a stabilizing mutation, or a destabilizing mutation) with respect to the parental molecule. In some embodiments, the affinity matured target antigen binding molecule has superior stability with respect to selected destabilizing agents, as that of a reference parental molecule specific to the same antigen. In some embodiments, the affinity matured target antigen binding molecule is identified in a process comprising panning of one or more pre-candidate target antigen binding molecules derived from one or more parental molecule, expressed in a phage display library, against a target protein. The pre-candidate target antigen binding molecule comprises, in some embodiments, amino acid substitutions in the variable regions, CDRs, or framework residues, relative to a parental molecule.

As used herein, “Phage display” refers to a technique by which variant polypeptides are displayed as fusion proteins to at least a portion of a coat protein on the surface of phage, e.g., filamentous phage, particles. A utility of phage display lies in the fact that large libraries of randomized protein variants can be rapidly and efficiently selected for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. See e.g. Wells and Lowman, Curr. Opin. Struct. Biol, 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that selection is on the basis of intrinsic ligand affinity, and phagemid vectors are used, which simplify DNA manipulations. See e.g., Lowman and Wells, Methods: A companion to Methods in Enzymology, 3:205-0216 (1991).

In some embodiments, the panning comprises using varying binding times and concentrations to identify target antigen binding molecules with increased or decreased on-rates, from pre-candidate target antigen binding molecules. In some embodiments, the panning comprises using varying wash times to identify target antigen binding molecules with increased or decreased off-rates, from pre-candidate target antigen molecules. In some embodiments, the panning comprises using both varying binding times and varying wash times. In some embodiments, one or more stabilizing mutations are combined to increase the stability of the affinity matured target antigen binding molecule, for example, by shuffling to create a second-stage combinatorial library from such mutants and conducting a second round of panning followed by a binding selection.

In some embodiments, the affinity matured target antigen binding molecule comprises an equivalent or better affinity to a target antigen protein (such as human target antigen protein) as that of a target antigen binding parental molecule, but that has reduced cross reactivity, or in some embodiments, increased cross reactivity, with selected substances, such as ligands, proteins, antigens, or the like, other than the target antigen epitope for which the target antigen binding parental molecule is specific, or is designed to be specific for. In regard to the latter, an affinity matured target antigen binding molecule, in some embodiments, is more successfully tested in animal models if the affinity matured target antigen binding molecule is reacted with both human target and the corresponding target of the animal model, e.g. mouse target or cynomolgus target. In some embodiments, the parental target antigen binding molecule binds to human target antigen with an affinity of about 500 nM or less, 400 nM or less, 300 nM or less, 200 nM or less, 100 nM or less, 50 nM or less, 10 nM or less, and to cynomolgus EpCAM with an affinity of about 500 nM or less, 400 nM or less, 300 nM or less, 200 nM or less, 100 nM or less, 50 nM or less, 15 nM or less, or 10 nM or less. In some embodiments, the affinity matured target antigen binding molecule, identified after one round of panning, binds to human target antigen with an affinity of about 5 nM or less, such as 1 nM or less, and to cynomolgus target antigen with an affinity of about 7.5 nM or less, such as 1 nM or less. In some embodiments, the affinity matured target antigen binding molecule, identified after two rounds of panning, binds to human target antigen with an affinity of about 2.5 nM or less, and to cynomolgus target antigen with an affinity of about 3.5 nM or less.

In some embodiments, the domains of the immune cell engaging protein are linked by one or more internal linkers. In certain embodiments, the internal linkers are “short,” i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, the internal linkers are “long,” i.e., consist of 15 to 25 amino acid residues. In some embodiments, the internal linkers consist of about 3 to about 15, for example 8, 9 or 10 contiguous amino acid residues. Regarding the amino acid composition of the internal linkers, peptides are selected with properties that confer flexibility to the target antigen binding proteins, do not interfere with the binding domains as well as resist cleavage from proteases. For example, glycine and serine residues generally provide protease resistance. Examples of internal linkers suitable for linking the domains in the target antigen binding proteins include but are not limited to (GS)ₙ (SEQ ID NO: 3859), (GGS)ₙ (SEQ ID NO: 3860), (GGGS)ₙ (SEQ ID NO: 3861), (GGSG)ₙ (SEQ ID NO: 3862), (GGSGG)ₙ (SEQ ID NO: 3863), (GGGGS)ₙ (SEQ ID NO: 3864), (GGGGG)ₙ (SEQ ID NO: 3865), or (GGG)ₙ (SEQ ID NO: 3866), wherein n is 1, 2, 3, 4, 5, 6, 7, 8, 9, or 10. In one embodiment, the linker is (GGGGSGGGGSGGGGSGGGGS)(SEQ ID NO: 3867), (GGGGSGGGGSGGGGS) (SEQ ID NO: 3868), LPETG (SEQ ID NO: 3869), (GGGGSGGGS) (SEQ ID NO: 3871) or SGGG (SEQ ID NO: 3872).

In some embodiments, the immune cell engaging protein described herein comprise a polypeptide having a sequence described in SEQ ID NOS: 3218-3462 and subsequences thereof. In some embodiments, the immune cell engaging protein comprises a polypeptide having at least 70%-95% or more identity to a sequence described in SEQ ID NO: 3218-3462. In some embodiments, the immune cell engaging protein comprises a polypeptide having at least 70%, 75%, 80%, 85%, 90%, 95%, 96%, 97%, 98%, 99%, or more identity to a sequence described in SEQ ID NO: 3218-3462. In some embodiments, the immune cell engaging protein has a sequence comprising at least a portion of a sequence described in SEQ ID NO: 3218-3462. In some embodiments, the immune cell engaging protein comprises a polypeptide comprising one or more of the sequences described in SEQ ID NO: 3218-3462.

In some embodiments, the immune cell engaging protein described herein comprise a polypeptide having a sequence described in SEQ ID NOS: 3255, 3340, 3376, and 3462 and subsequences thereof. In some embodiments, the immune cell engaging protein comprises a polypeptide having at least 70%-95% or more identity to a sequence described in SEQ ID NO: 3255, 3340, 3376, and 3462. In some embodiments, the immune cell engaging protein comprises a polypeptide having at least 70%, 75%, 80%, 85%, 90%, 95%, 96%, 97%, 98%, 99%, or more identity to a sequence described in SEQ ID NO: 3255, 3340, 3376, and 3462. In some embodiments, the immune cell engaging protein has a sequence comprising at least a portion of a sequence described in SEQ ID NO: 3255, 3340, 3376, and 3462. In some embodiments, the immune cell engaging protein comprises a polypeptide comprising one or more of the sequences described in SEQ ID NO: 3255, 3340, 3376, and 3462.

### CD19 Binding Domain

Described herein are immune cell engaging proteins that comprise a CD19 binding domain as the target antigen binding domain, pharmaceutical compositions thereof, as well as nucleic acids, recombinant expression vectors and host cells for making such proteins thereof. Also provided are methods of using the disclosed proteins comprising a CD19 binding domain of this disclosure, in the prevention, and/or treatment of diseases, conditions and disorders.

The CD19 binding domain, in some embodiments, are specific for CD19 expressed on cell surface. The anti-CD19 antibodies disclosed herein, in some instances, show high binding affinity to CD19 (e.g., cell-surface CD19), high stability, and/or bind to different CD19 epitopes compared to FMC63, which is an anti-CD19 scFv. CD19 is a 95 kDa transmembrane glycoprotein expressed primarily on B lineage cells and follicular dendritic cells. It is a member of the immunoglobulin super family. CD19 molecules from various species are well known in the art. For example, the amino acid sequence of human CD19 can be found under GenBank accession NO: AAA69966. CD19 plays essential roles in B cell malignancies and autoimmunity. CD19 is reported to be expressed on the surface of cancer cells in 90% of acute lymphoblastic leukemia (ALL) patients, as well as on cancer cells of B-cell non-Hodgkin's lymphoma (NHL) and chronic lymphocytic leukemia (CLL) patients. Therefore, CD19 has been considered as a promising target for immunotherapy of cancers of B cell lineage. See, e.g., Stanciu-Herrera et al., Leuk Res. 2008; 32:625-32; and Le Gall et al., FEBS Lett. 1999; 453:164-8.

In some embodiments, the CD19 binding domain comprises a sequence that is at least about 70% to about 99% or more identical to a sequence selected from the group consisting of SEQ ID NOS: 3771-3792. In some embodiments, the CD19 binding domain comprises a sequence that is at least about 70%, 75%, 80%, 85%, 90%, 95%, 96%, 97%, 98%, 99%, or 100% identical to a sequence selected from the group consisting of SEQ ID NOS: 3771-3792. In some embodiments, the CD19 binding domain comprises a sequence selected from the group consisting of SEQ ID NOS: 3771-3792.

### CD20 Binding Domain

Described herein are immune cell engaging proteins that comprise a CD20 binding domain as the target antigen binding domain, pharmaceutical compositions thereof, as well as nucleic acids, recombinant expression vectors and host cells for making such proteins thereof. Also provided are methods of using the disclosed proteins comprising a CD20 binding domain of this disclosure, in the prevention, and/or treatment of diseases, conditions and disorders.

CD20 molecule is a non-glycosylated phosphoprotein specifically labeled on the surface of human lymphocyte subgroup (B cell group). It consists of 297 amino acids with a molecular weight of 33-37 kDa, and is expressed on the surface of more than 95% of B cells. CD20 molecule exists in both normal B cells and malignant cells, and is especially expressed in more than 90% of B-cell non-Hodgkin's lymphoma. The CD20 molecule has four transmembrane regions, and the amino terminus and the carboxy terminus are located on the inner side of the plasma membrane. Between the third transmembrane region and the fourth transmembrane region, there is a loop region composed of 43 amino acid residues, which constitutes the main epitope. The CD20 antigen molecule is relatively exposed and accessible. When CD20 is close to each other under the action of antibodies, the polymer formed by cross-linking or even super-crosslinking functions as a calcium ion channel, allowing extracellular calcium ions to flow into the cells; in addition, the tyrosine protein kinases of the Src family activate each other due to proximity. Signaling pathways are initiated and endogenous calcium stores are mobilized, both of which lead to an increase in intracellular calcium ion concentration and then affect the operation of cell cycle, regulate cell proliferation and differentiation and even lead to the occurrence of apoptosis. Although the actual role of CD20 in promoting the proliferation and differentiation of B cells is not clear, CD20 provides an important target for antibody-mediated therapy, which can be used for controlling the B cells involved in cancers and autoimmune diseases.

In some embodiments, the CD20 binding domain comprises a sequence that is at least about 70% to about 99% or more identical to a sequence selected from the group consisting of SEQ ID NOS: 3793-3808 and 3880. In some embodiments, the CD20 binding domain comprises a sequence that is at least about 70%, 75%, 80%, 85%, 90%, 95%, 96%, 97%, 98%, 99%, or 100% identical to a sequence selected from the group consisting of SEQ ID NOS: 3793-3808 and 3880. In some embodiments, the CD20 binding domain comprises a sequence of SEQ ID NO: 3793-3808 and 3880.

### CD33 Binding Domain

Described herein are immune cell engaging proteins that comprise a CD33 binding domain as the target antigen binding domain, pharmaceutical compositions thereof, as well as nucleic acids, recombinant expression vectors and host cells for making such proteins thereof. Also provided are methods of using the disclosed proteins comprising a CD33 binding domain of this disclosure, in the prevention, and/or treatment of diseases, conditions and disorders.

CD33 (also known as Siglec-3, SIGLEC3, gp67, p67) is a 67 kDa plasma membrane protein that binds to sialic acid and is a member of the sialic acid-binding Ig-related lectin (SIGLEC) family of proteins. Siglec proteins are thought to be involved in diverse biological processes such as hematopoiesis, neuronal development and immunity (Vinson et al., J Biol. Chem. 271:9267-9272 (1996)). Studies also suggest that Siglec proteins mediate cell adhesion/cell signaling through recognition of sialylated cell surface glycans (Kelm et al., Glycoconj. J. 13:913-926 (1996); Kelm et al., Eur. J Biochem. 255:663-672 (1998); Vinson et al., J Biol. Chem. 271:9267-9272 (1996)). The extracellular portion of CD33 contains two immunoglobulin domains (one IgV domain and one IgC2 domain). The IgV domain is distal to the membrane whereas the IgC2 domain is proximal to the membrane. The intracellular portion of CD33 contains immunoreceptor tyrosine-based inhibitory motifs (ITIMs). In the immune response, CD33 may act as an inhibitory receptor upon ligand induced tyrosine phosphorylation by recruiting cytoplasmic phosphatase(s) that block signal transduction through dephosphorylation of signaling molecules.

CD33 is known to be expressed on myeloid cells. CD33 expression has also been reported on a number of malignant cells. Anti-CD33 agents are generally allocated in four groups: naked antibodies, antibody toxin conjugates, radionuclide conjugates, and bispecific antibodies. Although CD33 has been targeted for treatment of cancer, e.g., acute myeloid leukemia, no effective CD33-targeted treatments are currently on the market. Existing anti-CD33 agents suffer from, inter alia, inferior tumor antigen binding avidity and short in vivo half-life.

In some embodiments, the CD33 binding domain comprises a sequence that is at least about 70% to about 99% or more identical to a sequence selected from the group consisting of SEQ ID NOS: 3809-3823. In some embodiments, the CD33 binding domain comprises a sequence that is at least about 70%, 75%, 80%, 85%, 90%, 95%, 96%, 97%, 98%, 99%, or 100% identical to a sequence selected from the group consisting of SEQ ID NOS: 3809-3823. In some embodiments, the CD33 binding domain comprises a sequence selected from the group consisting of SEQ ID NOS: 3809-3823.

### Prostate Specific Membrane Antigen (PSMA) Binding Proteins

Described herein are immune cell engaging proteins that comprise a PSMA binding domain as the target antigen binding domain, pharmaceutical compositions thereof, as well as nucleic acids, recombinant expression vectors and host cells for making such proteins thereof. Also provided are methods of using the disclosed proteins comprising a PSMA binding domain of this disclosure, in the prevention, and/or treatment of diseases, conditions and disorders.

PSMA is a 100 kD Type II membrane glycoprotein expressed in prostate tissues having sequence identity with the transferrin receptor with NAALADase activity. PSMA is expressed in increased amounts in prostate cancer, and elevated levels of PSMA are also detectable in the sera of these patients. PSMA expression increases with disease progression, becoming highest in metastatic, hormone-refractory disease for which there is no present therapy.

In some embodiments, the PSMA binding domain comprises the following formula: f1-r1-f2-r2-f3-r3-f4, wherein r1, r2, and r3 are complementarity determining regions CDR1, CDR2, and CDR3, respectively, and f1, f2, f3, and f4 are framework residues, and wherein r1 comprises SEQ ID NO: 462, SEQ ID NO: 463, SEQ ID NO: 464, or SEQ ID NO: 465, r2 comprises SEQ ID NO: 466, SEQ ID NO: 467, SEQ ID NO: 468, SEQ ID NO: 469, SEQ ID NO: 470, SEQ ID NO: 471, SEQ ID NO: 472, or SEQ ID NO: 473, and r3 comprises SEQ ID NO: 474, or SEQ ID NO: 475.

In some embodiments, PSMA binding domains described herein comprise a polypeptide having a sequence described in SEQ ID NO: 462-489 and subsequences thereof. In some embodiments, the PSMA binding domain comprises a polypeptide having at least 70%-95% or more homology to a sequence described in SEQ ID NO: 462-489. In some embodiments, the PSMA binding domain comprises a polypeptide having at least 70%, 75%, 80%, 85%, 90%, 95%, 96%, 97%, 98%, 99%, or more identity to a sequence described in SEQ ID NO: 462-489. In some embodiments, the PSMA binding domain has a sequence comprising at least a portion of a sequence described in SEQ ID NO: 462-489. In some embodiments, the HSA binding domain comprises a polypeptide comprising one or more of the sequences described in SEQ ID NO: 462-489.

In some embodiments, PSMA binding domains described herein comprise a single domain antibody with a CDR1 comprising SEQ ID NO: 462-465. In some embodiments, PSMA binding domains described herein comprise a single domain antibody with a CDR2 comprising SEQ ID NO: 466-473. In some embodiments, PSMA binding domains described herein comprise a single domain antibody with a CDR3 comprising SEQ ID NO: 474 and 475.

### Mesothelin (MSLN) Binding Proteins

Described herein are immune cell engaging proteins that comprise an MSLN binding domain as the target antigen binding domain, pharmaceutical compositions thereof, as well as nucleic acids, recombinant expression vectors and host cells for making such proteins thereof. Also provided are methods of using the disclosed proteins comprising an MSLN binding domain of this disclosure, in the prevention, and/or treatment of diseases, conditions and disorders.

MSLN is a GPI-linked membrane bound tumor antigen. MSLN is overexpressed ovarian, pancreatic, lung and triple-negative breast cancers and mesothelioma. Normal tissue expression of MSLN is restricted to single-cell, mesothelial layers lining the pleural, pericardial, and peritoneal cavities. Overexpression of MSLN is associated with poor prognosis in lung adenocarcinoma and triple-negative breast cancer. MSLN has been used as cancer antigen for numerous modalities, including immunotoxins, vaccines, antibody drug conjugates and CAR-T cells. Early signs of clinical efficacy have validated MSLN as a target, but therapies with improved efficacy are needed to treat MSLN-expressing cancers.

Mesothelin is a glycoprotein present on the surface of cells of the mesothelial lining of the peritoneal, pleural and pericardial body cavities. The mesothelin gene (MSLN) encodes a 71 kD precursor protein that is processed to a 40 kD protein termed mesothelin, which is a glycosyl-phosphatidylinositol-anchored glycoprotein present on the cell surface (Chang, et al., Proc Natl Acad Sci USA (1996) 93:136-40). The mesothelin cDNA was cloned from a library prepared from the HPC-Y5 cell line (Kojima et al. (1995) J. Biol. Chem. 270:21984-21990). The cDNA also was cloned using the monoclonal antibody K1, which recognizes mesotheliomas (Chang and Pastan (1996) Proc. Natl. Acad. Sci. USA 93:136-40). Mesothelin is a differentiation antigen whose expression in normal human tissues is limited to mesothelial cells lining the body cavity, such as the pleura, pericardium and peritoneum. Mesothelin is also highly expressed in several different human cancers, including mesotheliomas, pancreatic adenocarcinomas, ovarian cancers, stomach and lung adenocarcinomas. (Hassan, et al., Eur J Cancer (2008) 44:46-53) (Ordonez, Am J Surg Pathol (2003) 27:1418-28; Ho, et al., Clin Cancer Res (2007) 13:1571-5). Mesothelin is overexpressed in a vast majority of primary pancreatic adenocarcinomas with rare and weak expression seen in benign pancreatic tissue. Argani P, et al. Clin Cancer Res. 2001; 7(12):3862-3868. Epithelial malignant pleural mesothelioma (MPM) universally expresses mesothelin while sarcomatoid MPM likely does not express mesothelin. Most serous epithelial ovarian carcinomas, and the related primary peritoneal carcinomas, express mesothelin.

Mesothelin can also be used a marker for diagnosis and prognosis of certain types of cancer because trace amounts of mesothelin can be detected in the blood of some patients with mesothelin-positive cancers (Cristaudo et al., Clin. Cancer Res. 13:5076-5081, 2007). It has been reported that mesothelin may be released into serum through deletion at its carboxyl terminus or by proteolytic cleavage from its membrane bound form (Hassan et al., Clin. Cancer Res. 10:3937-3942, 2004). An increase in the soluble form of mesothelin was detectable several years before malignant mesotheliomas occurred among workers exposed to asbestos (Creaney and Robinson, Hematol. Oncol. Clin. North Am. 19:1025-1040, 2005). Furthermore, patients with ovarian, pancreatic, and lung cancers also have elevated soluble mesothelin in serum (Cristaudo et al., Clin. Cancer Res. 13:5076-5081, 2007; Hassan et al., Clin. Cancer Res. 12:447-453, 2006; Croso et al., Cancer Detect. Prev. 30:180-187, 2006). Accordingly, mesothelin is an appropriate target for methods of disease prevention or treatment and there is a need for effective antibodies specific for mesothelin.

It has been shown that cell surface mature mesothelin comprises three distinct domains, namely Regions I (comprising residues 296-390), II (comprising residues 391-486), and III (comprising residue 487-598). (Tang et al., A human single-domain antibody elicits potent antitumor activity by targeting an epitope in mesothelin close to the cancer cell surface, Mol. Can. Therapeutics, 12(4): 416-426, 2013). The first antibodies generated against mesothelin for therapeutic intervention were designed to interfere with the interaction between mesothelin and CA-125. Phage display identified the Fv SS, which was affinity optimized and used to generate a recombinant immunotoxin targeting mesothelin, SS1P. The MORAb-009 antibody amatuximab, which also uses SS1, recognizes a non-linear epitope in the amino terminal 64 amino acids of mesothelin, within region I. The SS1 Fv was also used to generate chimeric antigen receptor-engineered T cells. Recently, new anti-mesothelin antibodies have been reported that recognize other regions of the mesothelin protein. The present disclosure provides, in certain embodiments, MSLN targeting immune cell engaging proteins containing binding domains which specifically bind to MSLN on the surface of tumor target cells.

In some embodiments, the MSLN binding domain binds to a protein comprising the sequence of SEQ ID NO: 3204. In some embodiments, the MSLN binding domain binds to a protein comprising a truncated sequence compared to SEQ ID NO: 3204.

In some embodiments, the MSLN binding domains disclosed herein recognize full-length mesothelin. In certain instances, the MSLN binding domains disclosed herein recognize an epitope in region I (comprising amino acid residues 296-390 of SEQ ID NO: 3204), region II (comprising amino acid residue 391-486 of SEQ ID NO: 3204), or region III (comprising amino acid residues 487-598 of SEQ ID NO: 3204) of mesothelin. It is contemplated that the MSLN binding domains of the present disclosure may, in some embodiments, recognize and bind to epitopes that are located outside regions I, II, or III of mesothelin. In yet other embodiments are disclosed MSLN binding domains that recognize and bind to an epitope different than the MORAb-009 antibody.

In some embodiments, the MSLN binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the MSLN binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the MSLN binding domain comprises an amino acid sequence selected from SEQ ID NOS: 607-650, or a sequence comprising at least 75% to about 95% or more (e.g., 96%, 97%, 98%, 99%, or more) identity to a sequence selected from the group consisting of SEQ ID NOS: 605-670.

In some embodiments, the CDR1 comprises the amino acid sequence as set forth in SEQ ID NO: 490 or a variant having one, two, three, four, five, six, seven, eight, nine, or ten amino acid substitutions in SEQ ID NO: 490. In some embodiments, the CDR2 comprises a sequence as set forth in SEQ ID NO: 3505 or a variant having one, two, three, four, five, six, seven, eight, nine, or ten amino acid substitutions in SEQ ID NO: 3505. In some embodiments, the CDR3 comprises a sequence as set forth in SEQ ID NO: 3506 or a variant having one, two, three, four, five, six, seven, eight, nine, or ten amino acid substitutions in SEQ ID NO: 3506.

In some embodiments, the CDR1 comprises the amino acid sequence as set forth in SEQ ID NO: 518 or a variant having one, two, three, four, five, six, seven, eight, nine, or ten amino acid substitutions in SEQ ID NO: 518. In some embodiments, the CDR2 comprises a sequence as set forth in SEQ ID NO: 3507 or a variant having one, two, three, four, five, six, seven, eight, nine, or ten amino acid substitutions in SEQ ID NO: 3507. In some embodiments, the CDR3 comprises a sequence as set forth in SEQ ID NO: 3508 or a variant having one, two, three, four, five, six, seven, eight, nine, or ten amino acid substitutions in SEQ ID NO: 3508.

In some embodiments, the CDR1 comprises the amino acid sequence as set forth in any one of SEQ ID NOS: 490-528 or a variant having one, two, three, four, five, six, seven, eight, nine, or ten amino acid substitutions in any one of SEQ ID NOS: 490-528. In some embodiments, the CDR2 comprises a sequence as set forth in any one of SEQ ID NOS: 529-567 or a variant having one, two, three, four, five, six, seven, eight, nine, or ten amino acid substitutions in any one of SEQ ID NOS: 529-567. In some embodiments, the CDR3 comprises a sequence as set forth in any one of SEQ ID NOS: 568-606 or a variant having one, two, three, four, five, six, seven, eight, nine, or ten amino acid substitutions in any one of SEQ ID NOS: 568-606.

In various embodiments, the MSLN binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 607-650.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 490, SEQ ID NO: 518, or any one of SEQ ID NOS: 490-528.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 3505, SEQ ID NO: 3507, or any one of SEQ ID NOS: 529-567.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 3506, SEQ ID NO: 3508, or any one of SEQ ID NOS: 568-606.

In some embodiments, any of the foregoing MSLN binding domains (e.g., anti-MSLN single domain antibodies of SEQ ID NOS: 607-650) are affinity peptide tagged for ease of purification. In some embodiments, the affinity peptide tag is six consecutive histidine residues, also referred to as 6X-his (SEQ ID NO: 3503).

In certain embodiments, the MSLN binding domains of the present disclosure preferentially bind membrane bound mesothelin over soluble mesothelin. Membrane bound mesothelin refers to the presence of mesothelin in or on the cell membrane surface of a cell that expresses mesothelin. Soluble mesothelin refers to mesothelin that is no longer on in or on the cell membrane surface of a cell that expresses or expressed mesothelin. In certain instances, the soluble mesothelin is present in the blood and/or lymphatic circulation in a subject. In one embodiment, the MSLN binding domains bind membrane-bound mesothelin at least 5 fold, 10 fold, 15 fold, 20 fold, 25 fold, 30 fold, 40 fold, 50 fold, 100 fold, 500 fold, or 1000 fold greater than soluble mesothelin. In one embodiment, the MSLN targeting immune cell engaging proteins of the present disclosure preferentially bind membrane-bound mesothelin 30 fold greater than soluble mesothelin. Determining the preferential binding of an antigen binding protein to membrane bound MSLN over soluble MSLN can be readily determined using assays well known in the art.

### B Cell Maturation Antigen (BCMA) Binding Proteins

Described herein are immune cell engaging proteins that comprise an BCMA binding domain as the target antigen binding domain, pharmaceutical compositions thereof, as well as nucleic acids, recombinant expression vectors and host cells for making such proteins thereof. Also provided are methods of using the disclosed proteins comprising an BCMA binding domain of this disclosure, in the prevention, and/or treatment of diseases, conditions and disorders.

B cell maturation antigen (BCMA, TNFRSF17, CD269) is a transmembrane protein belonging to the tumor necrosis family receptor (TNFR) super family that is primarily expressed on terminally differentiated B cells. BCMA expression is restricted to the B cell lineage and mainly present on plasma cells and plasmablasts and to some extent on memory B cells, but virtually absent on peripheral and naive B cells. BCMA is also expressed on multiple myeloma (MM) cells, on leukemia cells and lymphoma cells.

BCMA was identified through molecular analysis of a t(4;16)(q26;p13) translocation found in a human intestinal T cell lymphoma and an in-frame sequence was mapped to the 16p13.1 chromosome band.

Human BCMA cDNA has an open reading frame of 552 bp that encodes a 184 amino acid polypeptide. The BCMA gene is organized into three exons that are separated by two introns, each flanked by GT donor and AG acceptor consensus splicing sites, and codes for a transcript of 1.2 kb. The structure of BCMA protein includes an integral transmembrane protein based on a central 24 amino acid hydrophobic region in an alpha-helix structure.

The murine BCMA gene is located on chromosome 16 syntenic to the human 16p13 region, and also includes three exons that are separated by two introns. The gene encodes a 185 amino acid protein. Murine BCMA mRNA is expressed as a 404 bp transcript at the highest levels in plasmacytoma cells (J558) and at modest levels in the A20 B cell lymphoma line. Murine BCMA mRNA transcripts have also been detected at low levels in T cell lymphoma (EL4, BW5147) and dendritic cell (CB1D6, D2SC1) lines in contrast to human cell lines of T cell and dendritic cell origin. The murine BCMA cDNA sequence has 69.3% nucleotide identity with the human BCMA cDNA sequence and slightly higher identity (73.7%) when comparing the coding regions between these two cDNA sequences. Mouse BCMA protein is 62% identical to human BCMA protein and, like human BCMA, contains a single hydrophobic region, which may be an internal transmembrane segment. The N-terminal 40 amino acid domain of both murine and human BCMA protein have six conserved cysteine residues, consistent with the formation of a cysteine repeat motif found in the extracellular domain of TNFRs. Similar to members of the TNFR superfamily, BCMA protein contains a conserved aromatic residue four to six residues C-terminal from the first cysteine.

BCMA is not expressed at the cell surface, but rather, is located on the Golgi apparatus. The amount of BCMA expression is proportional to the stage of cellular differentiation (highest in plasma cells).

BCMA is involved in B cell development and homeostasis due to its interaction with its ligands BAFF (B cell activating factor, also designated as TALL-1 or TNFSF13B) and APRIL (A proliferation inducing ligand). BCMA regulates different aspects of humoral immunity, B cell development and homeostasis along with its family members TACI (transmembrane activator and cyclophilin ligand interactor) and BAFF-R (B cell activation factor receptor, also known as tumor necrosis factor receptor superfamily member 13C). Expression of BCMA appears rather late in B cell differentiation and contributes to the long-term survival of plasmablasts and plasma cells in the bone marrow. BCMA also supports growth and survival of multiple myeloma (MM) cells. BCMA is mostly known for its functional activity in mediating the survival of plasma cells that maintain long-term humoral immunity.

The present disclosure provides, in certain embodiments, single domain proteins which specifically bind to BCMA on the surface of tumor target cells.

In some embodiments, the BCMA binding domain binds to a protein comprising the sequence of SEQ ID NO: 3201, 3202 or 3203. In some embodiments, the BCMA binding domain binds to a protein comprising a truncated sequence compared to SEQ ID NO: 3201, 3202 or 3203.

In some embodiments, the BCMA binding protein of the present disclosure is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The r1 residues of the BCMA binding protein of the present disclosure comprise, for example, amino acid residues 26, 27, 28, 29, 30, 31, 32, 33 and 34; the r2 residues of the BCMA binding protein of the present disclosure comprise, for example, amino acid residues, for example, 50, 51, 52, 53, 54, 55, 56, 57, 58, 59, 60, 61, 62 and 63; and the r3 residues of the BCMA binding protein of the present disclosure comprise, for example, amino acid residues, for example, 97, 98, 99, 100, 101, 102, 103, 104, 105, 106, 107 and 108. In some embodiments, the BCMA binding protein comprises an amino acid sequence selected from SEQ ID NOS: 346-460, or a sequence comprising at least 75% to about 95% or more (e.g., 96%, 97%, 98%, 99%, or more) identity to a sequence selected from the group consisting of SEQ ID NOS: 346-460.

In some embodiments, an exemplary CDR1 comprises the amino acid sequence as set forth in SEQ ID NO: 1-115. In some embodiments, another exemplary CDR2 comprises the amino acid sequence as set forth in SEQ ID NO: 116-230. In some embodiments, another exemplary CDR3 comprises the amino acid sequence as set forth in SEQ ID NO: 231-345.

In various embodiments, the BCMA binding protein of the present disclosure has a CDR1 that has an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 1-115.

In various embodiments, the BCMA binding protein of the present disclosure has a CDR2 that has an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 116-230.

In various embodiments, a complementarity determining region of the BCMA binding protein of the present disclosure has a CDR3 that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 231-345.

In various embodiments, a BCMA binding protein of the present disclosure has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 346-460.

In some embodiments, the BCMA binding protein is a single domain antibody comprising the sequence of SEQ ID NO: 346. In some embodiments, the BCMA binding protein is a single domain antibody comprising the sequence of SEQ ID NO: 347. In some embodiments, the BCMA binding protein is a single domain antibody comprising the sequence of SEQ ID NO: 348. In some embodiments, the BCMA binding protein is a single domain antibody comprising the sequence of SEQ ID NO: 349. In some embodiments, the BCMA binding protein is a single domain antibody comprising the sequence of SEQ ID NO: 350. In some embodiments, the BCMA binding protein is a single domain antibody comprising the sequence of SEQ ID NO: 351. In some embodiments, the BCMA binding protein is a single domain antibody comprising the sequence of SEQ ID NO: 352. In some embodiments, the BCMA binding protein is a single domain antibody comprising the sequence of SEQ ID NO: 353. In some embodiments, the BCMA binding protein is a single domain antibody comprising the sequence of SEQ ID NO: 354. In some embodiments, the BCMA binding protein is a single domain antibody comprising the sequence of SEQ ID NO: 355. In some embodiments, the BCMA binding protein is a single domain antibody comprising the sequence of SEQ ID NO: 356. In some embodiments, the BCMA binding protein is a single domain antibody comprising the sequence of SEQ ID NO: 357. In some embodiments, the BCMA binding protein is a single domain antibody comprising the sequence of SEQ ID NO: 358. In some embodiments, the BCMA binding protein is a single domain antibody comprising the sequence of SEQ ID NO: 359.

In some embodiments, the BCMA binding protein is a single domain antibody comprising the sequence of SEQ ID NO: 360. In some embodiments, the BCMA binding protein is a single domain antibody comprising the sequence of SEQ ID NO: 361. In some embodiments, the BCMA binding protein is a single domain antibody comprising the sequence of SEQ ID NO: 362. In some embodiments, the BCMA binding protein is a single domain antibody comprising the sequence of SEQ ID NO: 363. In some embodiments, the BCMA binding protein is a single domain antibody comprising the sequence of SEQ ID NO: 364. In some embodiments, the BCMA binding protein is a single domain antibody comprising the sequence of SEQ ID NO: 365. In some embodiments, the BCMA binding protein is a single domain antibody comprising the sequence of SEQ ID NO: 366. In some embodiments, the BCMA binding protein is a single domain antibody comprising the sequence of SEQ ID NO: 367. In some embodiments, the BCMA binding protein is a single domain antibody comprising the sequence of SEQ ID NO: 368. In some embodiments, the BCMA binding protein is a single domain antibody comprising the sequence of SEQ ID NO: 369.

In some embodiments, the BCMA binding protein is a single domain antibody comprising the sequence of SEQ ID NO: 370. In some embodiments, the BCMA binding protein is a single domain antibody comprising the sequence of SEQ ID NO: 371. In some embodiments, the BCMA binding protein is a single domain antibody comprising the sequence of SEQ ID NO: 372. In some embodiments, the BCMA binding protein is a single domain antibody comprising the sequence of SEQ ID NO: 373. In some embodiments, the BCMA binding protein is a single domain antibody comprising the sequence of SEQ ID NO: 374. In some embodiments, the BCMA binding protein is a single domain antibody comprising the sequence of SEQ ID NO: 375. In some embodiments, the BCMA binding protein is a single domain antibody comprising the sequence of SEQ ID NO: 376. In some embodiments, the BCMA binding protein is a single domain antibody comprising the sequence of SEQ ID NO: 377. In some embodiments, the BCMA binding protein is a single domain antibody comprising the sequence of SEQ ID NO: 378. In some embodiments, the BCMA binding protein is a single domain antibody comprising the sequence of SEQ ID NO: 379.

In some embodiments, the BCMA binding protein is a single domain antibody comprising the sequence of SEQ ID NO: 380. In some embodiments, the BCMA binding protein is a single domain antibody comprising the sequence of SEQ ID NO: 381. In some embodiments, the BCMA binding protein is a single domain antibody comprising the sequence of SEQ ID NO: 382. In some embodiments, the BCMA binding protein is a single domain antibody comprising the sequence of SEQ ID NO: 383. In some embodiments, the BCMA binding protein is a single domain antibody comprising the sequence of SEQ ID NO: 384. In some embodiments, the BCMA binding protein is a single domain antibody comprising the sequence of SEQ ID NO: 385. In some embodiments, the BCMA binding protein is a single domain antibody comprising the sequence of SEQ ID NO: 386. In some embodiments, the BCMA binding protein is a single domain antibody comprising the sequence of SEQ ID NO: 387. In some embodiments, the BCMA binding protein is a single domain antibody comprising the sequence of SEQ ID NO: 388. In some embodiments, the BCMA binding protein is a single domain antibody comprising the sequence of SEQ ID NO: 389.

In some embodiments, the BCMA binding protein is a single domain antibody comprising the sequence of SEQ ID NO: 390. In some embodiments, the BCMA binding protein is a single domain antibody comprising the sequence of SEQ ID NO: 391. In some embodiments, the BCMA binding protein is a single domain antibody comprising the sequence of SEQ ID NO: 392. In some embodiments, the BCMA binding protein is a single domain antibody comprising the sequence of SEQ ID NO: 393. In some embodiments, the BCMA binding protein is a single domain antibody comprising the sequence of SEQ ID NO: 394. In some embodiments, the BCMA binding protein is a single domain antibody comprising the sequence of SEQ ID NO: 395. In some embodiments, the BCMA binding protein is a single domain antibody comprising the sequence of SEQ ID NO: 396. In some embodiments, the BCMA binding protein is a single domain antibody comprising the sequence of SEQ ID NO: 397. In some embodiments, the BCMA binding protein is a single domain antibody comprising the sequence of SEQ ID NO: 398. In some embodiments, the BCMA binding protein is a single domain antibody comprising the sequence of SEQ ID NO: 399.

In some embodiments, the BCMA binding protein is a single domain antibody comprising the sequence of SEQ ID NO: 400. In some embodiments, the BCMA binding protein is a single domain antibody comprising the sequence of SEQ ID NO: 401. In some embodiments, the BCMA binding protein is a single domain antibody comprising the sequence of SEQ ID NO: 402. In some embodiments, the BCMA binding protein is a single domain antibody comprising the sequence of SEQ ID NO: 403. In some embodiments, the BCMA binding protein is a single domain antibody comprising the sequence of SEQ ID NO: 404. In some embodiments, the BCMA binding protein is a single domain antibody comprising the sequence of SEQ ID NO: 405. In some embodiments, the BCMA binding protein is a single domain antibody comprising the sequence of SEQ ID NO: 406. In some embodiments, the BCMA binding protein is a single domain antibody comprising the sequence of SEQ ID NO: 407. In some embodiments, the BCMA binding protein is a single domain antibody comprising the sequence of SEQ ID NO: 408. In some embodiments, the BCMA binding protein is a single domain antibody comprising the sequence of SEQ ID NO: 409.

In some embodiments, the BCMA binding protein is a single domain antibody comprising the sequence of SEQ ID NO: 410. In some embodiments, the BCMA binding protein is a single domain antibody comprising the sequence of SEQ ID NO: 411. In some embodiments, the BCMA binding protein is a single domain antibody comprising the sequence of SEQ ID NO: 412. In some embodiments, the BCMA binding protein is a single domain antibody comprising the sequence of SEQ ID NO: 413. In some embodiments, the BCMA binding protein is a single domain antibody comprising the sequence of SEQ ID NO: 414. In some embodiments, the BCMA binding protein is a single domain antibody comprising the sequence of SEQ ID NO: 415. In some embodiments, the BCMA binding protein is a single domain antibody comprising the sequence of SEQ ID NO: 416. In some embodiments, the BCMA binding protein is a single domain antibody comprising the sequence of SEQ ID NO: 417. In some embodiments, the BCMA binding protein is a single domain antibody comprising the sequence of SEQ ID NO: 418. In some embodiments, the BCMA binding protein is a single domain antibody comprising the sequence of SEQ ID NO: 419.

In some embodiments, the BCMA binding protein is a single domain antibody comprising the sequence of SEQ ID NO: 420. In some embodiments, the BCMA binding protein is a single domain antibody comprising the sequence of SEQ ID NO: 421. In some embodiments, the BCMA binding protein is a single domain antibody comprising the sequence of SEQ ID NO: 422. In some embodiments, the BCMA binding protein is a single domain antibody comprising the sequence of SEQ ID NO: 423. In some embodiments, the BCMA binding protein is a single domain antibody comprising the sequence of SEQ ID NO: 424. In some embodiments, the BCMA binding protein is a single domain antibody comprising the sequence of SEQ ID NO: 425. In some embodiments, the BCMA binding protein is a single domain antibody comprising the sequence of SEQ ID NO: 426. In some embodiments, the BCMA binding protein is a single domain antibody comprising the sequence of SEQ ID NO: 427. In some embodiments, the BCMA binding protein is a single domain antibody comprising the sequence of SEQ ID NO: 428. In some embodiments, the BCMA binding protein is a single domain antibody comprising the sequence of SEQ ID NO: 429.

In some embodiments, the BCMA binding protein is a single domain antibody comprising the sequence of SEQ ID NO: 430. In some embodiments, the BCMA binding protein is a single domain antibody comprising the sequence of SEQ ID NO: 431. In some embodiments, the BCMA binding protein is a single domain antibody comprising the sequence of SEQ ID NO: 432. In some embodiments, the BCMA binding protein is a single domain antibody comprising the sequence of SEQ ID NO: 433. In some embodiments, the BCMA binding protein is a single domain antibody comprising the sequence of SEQ ID NO: 434. In some embodiments, the BCMA binding protein is a single domain antibody comprising the sequence of SEQ ID NO: 435. In some embodiments, the BCMA binding protein is a single domain antibody comprising the sequence of SEQ ID NO: 436. In some embodiments, the BCMA binding protein is a single domain antibody comprising the sequence of SEQ ID NO: 437. In some embodiments, the BCMA binding protein is a single domain antibody comprising the sequence of SEQ ID NO: 438. In some embodiments, the BCMA binding protein is a single domain antibody comprising the sequence of SEQ ID NO: 439.

In some embodiments, the BCMA binding protein is a single domain antibody comprising the sequence of SEQ ID NO: 440. In some embodiments, the BCMA binding protein is a single domain antibody comprising the sequence of SEQ ID NO: 441. In some embodiments, the BCMA binding protein is a single domain antibody comprising the sequence of SEQ ID NO: 442. In some embodiments, the BCMA binding protein is a single domain antibody comprising the sequence of SEQ ID NO: 443. In some embodiments, the BCMA binding protein is a single domain antibody comprising the sequence of SEQ ID NO: 444. In some embodiments, the BCMA binding protein is a single domain antibody comprising the sequence of SEQ ID NO: 445. In some embodiments, the BCMA binding protein is a single domain antibody comprising the sequence of SEQ ID NO: 446. In some embodiments, the BCMA binding protein is a single domain antibody comprising the sequence of SEQ ID NO: 447. In some embodiments, the BCMA binding protein is a single domain antibody comprising the sequence of SEQ ID NO: 448. In some embodiments, the BCMA binding protein is a single domain antibody comprising the sequence of SEQ ID NO: 449.

In some embodiments, the BCMA binding protein is a single domain antibody comprising the sequence of SEQ ID NO: 450. In some embodiments, the BCMA binding protein is a single domain antibody comprising the sequence of SEQ ID NO: 451. In some embodiments, the BCMA binding protein is a single domain antibody comprising the sequence of SEQ ID NO: 452. In some embodiments, the BCMA binding protein is a single domain antibody comprising the sequence of SEQ ID NO: 453. In some embodiments, the BCMA binding protein is a single domain antibody comprising the sequence of SEQ ID NO: 454. In some embodiments, the BCMA binding protein is a single domain antibody comprising the sequence of SEQ ID NO: 455. In some embodiments, the BCMA binding protein is a single domain antibody comprising the sequence of SEQ ID NO: 456. In some embodiments, the BCMA binding protein is a single domain antibody comprising the sequence of SEQ ID NO: 457. In some embodiments, the BCMA binding protein is a single domain antibody comprising the sequence of SEQ ID NO: 458. In some embodiments, the BCMA binding protein is a single domain antibody comprising the sequence of SEQ ID NO: 459. In some embodiments, the BCMA binding protein is a single domain antibody comprising the sequence of SEQ ID NO: 460.

In some embodiments, any of the foregoing BCMA binding domains are affinity peptide tagged for ease of purification. In some embodiments, the affinity peptide tag is six consecutive histidine residues, also referred to as a His tag or 6X-his (His-His-His-His-His-His; SEQ ID NO: 3503).

In certain embodiments, the BCMA binding domains of the present disclosure preferentially bind membrane bound BCMA over soluble BCMA. Membrane bound BCMA refers to the presence of BCMA in or on the cell membrane surface of a cell that expresses BCMA. Soluble BCMA refers to BCMA that is no longer on in or on the cell membrane surface of a cell that expresses or expressed BCMA. In certain instances, the soluble BCMA is present in the blood and/or lymphatic circulation in a subject. In one embodiment, the BCMA binding domains bind membrane-bound BCMA at least 5 fold, 10 fold, 15 fold, 20 fold, 25 fold, 30 fold, 40 fold, 50 fold, 100 fold, 500 fold, or 1000 fold greater than soluble BCMA. In one embodiment, the BCMA targeting immune cell engaging proteins of the present disclosure preferentially bind membrane-bound BCMA 30 fold greater than soluble BCMA. Determining the preferential binding of an antigen binding protein to membrane bound BCMA over soluble BCMA can be readily determined using assays well known in the art.

### Delta-Like Ligand 3 (DLL3) Binding Proteins

DLL3 (also known as Delta-like Ligand 3 or SCDO1) is a member of the Delta-like family of Notch DSL ligands. Representative DLL3 protein orthologs include, but are not limited to, human (Accession NOS: NP_058637 and NP_982353), chimpanzee (Accession NO: XP_003316395), mouse (Accession NO: NP_031892), and rat (Accession NO: NP_446118). In humans, the DLL3 gene consists of 8 exons spanning 9.5 kbp located on chromosome 19q13. Alternate splicing within the last exon gives rise to two processed transcripts, one of 2389 bases (Accession NO: NM_016941) and one of 2052 bases (Accession NO: NM_203486). The former transcript encodes a 618 amino acid protein (Accession NO: NP_058637), whereas the latter encodes a 587 amino acid protein (Accession NO: NP_982353). These two protein isoforms of DLL3 share overall 100% identity across their extracellular domains and their transmembrane domains, differing only in that the longer isoform contains an extended cytoplasmic tail containing 32 additional residues at the carboxy terminus of the protein. The extracellular region of the DLL3 protein, comprises six EGF-like domains, the single DSL domain and the N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3. Each of the EGF-like domains, the DSL domain and the N-terminal domain comprise part of the DLL3 protein as defined by a distinct amino acid sequence. The EGF-like domains are termed, in some embodiments, as EGF1 to EGF6 with EGF1 being closest to the N-terminal portion of the protein. In general, DSL ligands are composed of a series of structural domains: a unique N-terminal domain, followed by a conserved DSL domain, multiple tandem epidermal growth factor (EGF)-like repeats, a transmembrane domain, and a cytoplasmic domain not highly conserved across ligands but one which contains multiple lysine residues that are potential sites for ubiquitination by unique E3 ubiquitin ligases. The DSL domain is a degenerate EGF-domain that is necessary but not sufficient for interactions with Notch receptors. Additionally, the first two EGF-like repeats of most DSL ligands contain a smaller protein sequence motif known as a DOS domain that co-operatively interacts with the DSL domain when activating Notch signaling.

In some embodiments, the disclosed DLL3 immune cell engaging proteins of this disclosure are generated, fabricated, engineered or selected so as to react with a selected domain, motif or epitope within a DLL3 protein. In some embodiments, the DLL3 targeting immune cell engaging protein binds to the DSL domain and, in some embodiments, binds to an epitope comprising G203, R205, P206 within the DSL domain.

The DLL3 binding domain of the DLL3 targeting immune cell engaging proteins of the present disclosure are, in some embodiments, engineered fabricated and/or selected to react with both isoform(s) of DLL3 or a single isoform of the protein or, conversely, comprise a pan-DLL binding domain that reacts or associates with at least one additional DLL family member in addition to DLL3. In some embodiments, the DLL3 binding domain, such as DLL3 binding domain are engineered, fabricated, and/or selected so that they react with domains (or epitopes therein) that are exhibited by DLL3 only or with domains that are at least somewhat conserved across multiple or all DLL family members.

In some embodiments the DLL3 binding domain associates or binds to a specific epitope, portion, motif or domain of DLL3. Both DLL3 isoforms incorporate an identical extracellular region comprising at least an N-terminal domain, a DSL (Delta/Serrate/lag-2) domain and six EGF-like domains (i.e., EGF1-EGF6). Accordingly, in certain embodiments the DLL3 binding domain binds or associate with the N-terminal domain of DLL3 (amino acids 27-175 in the mature protein) while in other embodiments the DLL3 binding domain associates with the DSL domain (amino acids 176-215) or epitope therein. In other aspects of the present disclosure the DLL3 binding domain associates or bind to a specific epitope located in a particular EGF-like domain of DLL3. In some embodiments, the DLL3 binding domain associates or binds to an epitope located in EGF1 (amino acids 216-249), EGF2 (amino acids 274-310), EGF3 (amino acids 312-351), EGF4 (amino acids 353-389), EGF5 (amino acids 391.427) or EGF6 (amino acids 429-465). In some embodiments, each of the aforementioned domains comprises more than one epitope and/or more than one bin. In some embodiments the DLL3 binding domain binds, reacts or associates with the DSL domain or an epitope therein. In other embodiments the DLL3 binding domain binds, reacts or associates with a particular EGF-like domain or an epitope therein. In some embodiments the DLL3 binding domain binds, reacts or associates with the N-terminal domain or an epitope therein.

In some embodiments, the DLL3 binding proteins of this disclosure, such as the DLL3 binding domain of the immune cell engaging proteins of this disclosure binds to the full length DLL3 protein or to a fragment thereof, such as epitope containing fragments within the full length DLL3 protein, as described above. In some cases, the epitope containing fragment comprises antigenic or immunogenic fragments and derivatives thereof of the DLL3 protein. Epitope containing fragments, including antigenic or immunogenic fragments, are, in some embodiments, 12 amino acids or more, 20 amino acids or more, 50 or 100 amino acids or more. The DLL3 fragments, in some embodiments, comprises 95% or more of the length of the full protein, 90% or more, 75% or 50% or 25% or 10% or more of the length of the full protein. In some embodiments, the epitope-containing fragments of DLL3 including antigenic or immunogenic fragments are capable of eliciting a relevant immune response in a patient. Derivatives of DLL3 include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the DLL3 sequence provided in SEQ ID NO: 3216 (UniProtKB Accession Q9NYJ7). In some embodiments, substitutions comprise conservative substitutions. Derivatives and variants of DLL3, in some examples, have essentially the same biological function as the DLL3 protein from which they are derived. For instance, derivatives and variants of DLL3 are, in some cases, comparably antigenic or immunogenic to the protein from which they are derived, have either the ligand-binding activity, or the active receptor-complex forming ability, or preferably both, of the protein from which they are derived, and have the same tissue distribution as DLL3.

In some embodiments, the DLL3 binding domain binds to a protein comprising the sequence of SEQ ID NO: 3216 (UniProtKB Accession Q9NYJ7). In some embodiments, the DLL3 binding domain binds to a protein comprising a truncated sequence compared to SEQ ID NO: 3216 (UniProtKB Accession Q9NYJ7). In some embodiments, the DLL3 binding domain binds to a protein comprising the sequence of SEQ ID NO: 3509 or SEQ ID NO: 3217 (which is the mature extracellular domain of a DLL3 protein). In some embodiments, the DLL3 binding domain binds to a protein comprising amino acids 47-492 of SEQ ID NO: 3509. In some embodiments, the DLL3 binding domain recognizes an epitope within amino acids 47-4492 of SEQ ID NO: 3509.

In another example of a substitution to create a variant anti-DLL3 antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the DLL3 binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the DLL3 binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the DLL3 binding domain comprises an amino acid sequence selected from SEQ ID NOS: 1308-1750. In some embodiments, CDR1 of the DLL3 binding domain comprises a sequence selected from SEQ ID NOS: 1751-2193, or one or more amino acid substitutions relative to a sequence selected from the group consisting of SEQ ID NOS: 1751-2193. In some embodiments, CDR2 comprises a sequence selected from the group consisting of SEQ ID NOS: 2194-2636, or one or more amino acid substitutions relative to a sequence selected from the group consisting of SEQ ID NOS: 2194-2636. In some embodiments, the CDR3 comprises a sequence selected from the group consisting of SEQ ID NOS: 2637-3080, or one or more substitutions relative to a sequence selected from SEQ ID NOS: 2637-3080.

In some embodiments, the CDR1 comprises an amino acid sequence selected from SEQ ID NOS: 1751-2193 or a variant having one, two, three, four, five, six, seven, eight, nine, or ten amino acid substitutions in an amino acid selected from SEQ ID NOS: 1751-2193. In some embodiments, the CDR2 comprises an amino acid sequence selected from SEQ ID NOS: 2194-2636 or a variant having one, two, three, four, five, six, seven, eight, nine, or ten amino acid substitutions in an amino acid sequence selected from SEQ ID NOS: 2194-2636. In some embodiments, the CDR3 comprises an amino acid sequence selected from SEQ ID NOS: 2637-3080 or a variant having one, two, three, four, five, six, seven, eight, nine, or ten amino acid substitutions in a sequence selected from SEQ ID NOS: 2637-3080.

In some embodiments, the CDR1 comprises an amino acid sequence selected from SEQ ID NOS: 1803-1836 or a variant having one, two, three, four, five, six, seven, eight, nine, or ten amino acid substitutions in an amino acid selected from SEQ ID NOS: 1803-1836. In some embodiments, the CDR2 comprises an amino acid sequence selected from SEQ ID NOS: 2246-2279 or a variant having one, two, three, four, five, six, seven, eight, nine, or ten amino acid substitutions in an amino acid sequence selected from SEQ ID NOS: 2246-2279. In some embodiments, the CDR3 comprises an amino acid sequence selected from SEQ ID NOS: 2689-2722 or a variant having one, two, three, four, five, six, seven, eight, nine, or ten amino acid substitutions in a sequence selected from SEQ ID NOS: 2689-2722.

In some embodiments, the CDR1 comprises an amino acid sequence selected from SEQ ID NOS: 1837-2117 or a variant having one, two, three, four, five, six, seven, eight, nine, or ten amino acid substitutions in an amino acid selected from SEQ ID NOS: 1837-2117. In some embodiments, the CDR2 comprises an amino acid sequence selected from SEQ ID NOS: 2280-2560 or a variant having one, two, three, four, five, six, seven, eight, nine, or ten amino acid substitutions in an amino acid sequence selected from SEQ ID NOS: 2280-2560. In some embodiments, the CDR3 comprises an amino acid sequence selected from SEQ ID NOS: 2723-3003 or a variant having one, two, three, four, five, six, seven, eight, nine, or ten amino acid substitutions in a sequence selected from SEQ ID NOS: 2723-3003.

In some embodiments, the CDR1 comprises an amino acid sequence selected from SEQ ID NOS: 2118-2193 or a variant having one, two, three, four, five, six, seven, eight, nine, or ten amino acid substitutions in an amino acid selected from SEQ ID NOS: 2118-2193. In some embodiments, the CDR2 comprises an amino acid sequence selected from SEQ ID NOS: 2561-2636 or a variant having one, two, three, four, five, six, seven, eight, nine, or ten amino acid substitutions in an amino acid sequence selected from SEQ ID NOS: 2561-2636. In some embodiments, the CDR3 comprises an amino acid sequence selected from SEQ ID NOS: 3004-3080 or a variant having one, two, three, four, five, six, seven, eight, nine, or ten amino acid substitutions in a sequence selected from SEQ ID NOS: 3004-3080.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 1308-1750. In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 1360-1393.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 1394-1674.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to SEQ ID No. 1375, or a sequence derived from SEQ ID NO: 1375.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to SEQ ID No. 1382, or a sequence derived from SEQ ID No. 1382.

In certain embodiments, the DLL3 binding domains of the present disclosure preferentially bind membrane bound DLL3 over soluble DLL3. Membrane bound DLL3 refers to the presence of DLL3 in or on the cell membrane surface of a cell that expresses DLL3. Soluble DLL3 refers to DLL3 that is no longer on in or on the cell membrane surface of a cell that expresses or expressed DLL3. In certain instances, the soluble DLL3 is present in the blood and/or lymphatic circulation in a subject. In one embodiment, the DLL3 binding proteins bind membrane-bound DLL3 at least 5 fold, 10 fold, 15 fold, 20 fold, 25 fold, 30 fold, 40 fold, 50 fold, 100 fold, 500 fold, or 1000 fold greater than soluble DLL3. In one embodiment, the antigen binding proteins of the present disclosure preferentially bind membrane-bound DLL3 30 fold greater than soluble DLL3. Determining the preferential binding of an antigen binding protein to membrane bound DLL3 over soluble DLL3 can be readily determined using assays well known in the art.

In some embodiments, any of the foregoing DLL3 binding domains (e.g., anti-DLL3 single domain antibodies of SEQ ID NOS: 1308-1750) are affinity peptide tagged for ease of purification. In some embodiments, the affinity peptide tag is six consecutive histidine residues, also referred to as 6X-his (SEQ ID NO: 3503).

In some embodiments, any of the foregoing DLL3 binding domains (e.g., anti-DLL3 single domain antibodies of SEQ ID NOS: 1308-1750) are affinity peptide tagged for ease of purification. In some embodiments, the affinity peptide tag is six consecutive histidine residues, also referred to as 6X-His (SEQ ID NO: 3503).

### Epidermal Growth Factor Receptor (EGFR) Binding Proteins

Described herein are immune cell engaging proteins that comprise an EGFR binding domain as the target antigen binding domain, pharmaceutical compositions thereof, as well as nucleic acids, recombinant expression vectors and host cells for making such proteins thereof. Also provided are methods of using the disclosed proteins comprising an EGFR binding domain of this disclosure, in the prevention, and/or treatment of diseases, conditions and disorders.

Epidermal growth factor receptor (EGFR) has been causally implicated in human malignancy. Abnormal activity of the Her family of receptors is involved with breast cancer. EGFR, Her-3, and Her-4 are frequently expressed in ovarian granulosa cell tumors (Leibl, S. et al., Gynecol Oncol 101:18-23 (2005). In particular, increased expression of EGFR has been observed in breast, bladder, lung, head, neck and stomach cancer as well as glioblastomas.

Increased EGFR receptor expression may be associated with increased production of a EGFR ligand, transforming growth factor alpha (TGF-α), by the same tumor cells resulting in receptor activation by an autocrine stimulatory pathway.

Cetuximab (ERBITUX™), an anti-EGFR antibody, has been associated with potentially life-threatening infusion reactions (Thomas, M., Clin J Oncol Nurs. 9(3):332-8 (2005)). Gefitinib (IRESSA™) and erlotinib (TARCEVA™), both EGFR specific small molecule inhibitors, are associated with a risk of interstitial lung disease (Sandler A, Oncology 20 (5 Suppl 2):35-40 (2006)). Individual patients may be predisposed to particular types of complications that affect the choice of drug treatment. There is a need for a greater choice of treatment options which allows physicians to select the therapeutic with the best side effect profile for an individual patient. The present disclosure provides novel polypeptides and protein therapeutics useful in methods of treatment, particularly for treatment of conditions associated with abnormal expression of EGFR.

Epidermal growth factor receptor (EGFR, also known as HER1 or ErbB1) is a member of the ErbB/HER family of type 1 receptor tyrosine kinases (RTKs). Other members of this family include ErbB2 (HER2 or Neu), ErbB3 (HER3) and ErbB4 (HER4). Known ligands for EGFR include epidermal growth factor (EGF) and transforming growth factor alpha (TGF-alpha). Ligand binding to EGFR is known to induce tyrosine phosphorylation and receptor dimerization with other ErbB family members.

RTKs such as EGFR function to allow cells to respond to diverse external stimuli. However, aberrant activation and/or overexpression of EGFR is associated with the development and progression of several human cancers. Accordingly, EGFR is a target for anti-cancer therapies. Approved drugs targeting EGFR include small molecule inhibitors such as gefitinib (IRESSA®) and erlotinib (TARCEVA®), and anti-EGFR antibodies such as cetuximab (ERBITUX®) and panitumumab (VECTIBIX®). Anti-EGFR antibodies are mentioned in, e.g., U.S. Pat. Nos. 4,943,533, 5,844,093, 7,060,808, 7,247,301, 7,595,378, 7,723,484, and 7,939,072.

There is still a need for having available further options for the treatment of diseases related to the overexpression of EGFR, including, but not limited to, renal cell carcinoma, pancreatic carcinoma, breast cancer, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer (e.g., EGFR-dependent non-small cell lung cancer), synovial sarcoma, thyroid cancer, or melanoma. The present disclosure provides, in certain embodiments, EGFR binding proteins, EGFR targeting immune cell engaging proteins containing EGFR binding domains which specifically bind to EGFR on the surface of tumor target cells.

In some embodiments, the EGFR binding domain of this disclosure binds to a protein comprising the sequence of SEQ ID NO: 3205 (UniProt Accession NO: Q504U8). In some embodiments, the EGFR binding domain binds to a protein comprising a truncated sequence compared to SEQ ID NO: 3205. In some embodiments, the EGFR binding domain binds to a protein comprising the sequence of SEQ ID NO: 3206 (UniProt Accession NO: Q01279). In some embodiments, the EGFR binding domain binds to a protein comprising a truncated sequence compared to SEQ ID NO: 3206. In some embodiments, the EGFR binding domain binds to a protein comprising the sequence of SEQ ID NO: 3207 (UniProt Accession NO: A0A2K5WK39). In some embodiments, the EGFR binding domain binds to a protein comprising a truncated sequence compared to SEQ ID NO: 3207.

In some embodiments, the EGFR binding domains disclosed herein recognize full-length EGFR. In certain instances, the EGFR binding domains disclosed herein recognize an epitope within EGFR, such as, in some cases the EGFR targeting immune cell engaging proteins interact with one or more amino acids found within the extracellular domain of human EGFR (e.g., within extracellular domain I, II, III, and/or IV). The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within the extracellular domain of EGFR. Alternatively, the epitope may consist of a plurality of non-contiguous amino acids (or amino acid sequences) located within the extracellular domain of EGFR.

In some embodiments, the EGFR binding proteins of this disclosure binds to the full-length EGFR protein or to a fragment thereof, such as epitope containing fragments within the full-length EGFR protein, as described above. In some cases, the epitope containing fragment comprises antigenic or immunogenic fragments and derivatives thereof of the EGFR protein. Epitope containing fragments, including antigenic or immunogenic fragments, are, in some embodiments, 12 amino acids or more, e.g., 20 amino acids or more, 50 or 100 amino acids or more. The EGFR fragments, in some embodiments, comprises 95% or more of the length of the full protein, 90% or more, 75% or 50% or 25% or 10% or more of the length of the full protein. In some embodiments, the epitope-containing fragments of EGFR including antigenic or immunogenic fragments are capable of eliciting a relevant immune response in a patient. Derivatives of EGFR include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the EGFR sequence provided in SEQ ID NO: 3205, 3206, or 3207.

In some embodiments, the EGFR binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the EGFR binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the EGFR binding domain comprises an amino acid sequence selected from SEQ ID NOS: 798-846.

In some embodiments, the EGFR binding domains described herein comprise a polypeptide having a sequence selected from SEQ ID NOS: 798-846, subsequences thereof, and variants thereof. In some embodiments, the EGFR binding protein comprises at least 70%-95% or more identity to a sequence selected from SEQ ID NOS: 798-846, subsequences thereof, and variants thereof. In some embodiments, the EGFR binding protein comprises at least 70%, 75%, 80%, 85%, 90%, 95%, 96%, 97%, 98%, 99%, or more identity to a sequence selected from SEQ ID NOS: 798-846, subsequences thereof, and variants thereof.

In some embodiments, the CDR1 comprises the amino acid sequence as set forth in any one of SEQ ID NOS: 651-699 or a variant having one, two, three, four, five, six, seven, eight, nine, or ten amino acid substitutions in any one of SEQ ID NOS: 651-699. In some embodiments, the CDR2 comprises a sequence as set forth in any one of SEQ ID NOS: 700-748 or a variant having one, two, three, four, five, six, seven, eight, nine, or ten amino acid substitutions in any one of SEQ ID NOS: 700-748. In some embodiments, the CDR3 comprises a sequence as set forth in any one of SEQ ID NOS: 148-196 or a variant having one, two, three, four, five, six, seven, eight, nine, or ten amino acid substitutions in any one of SEQ ID NOS: 749-797.

In various embodiments, the EGFR binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 798-846.

In various embodiments, a complementarity determining region of the EGFR binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence set forth in any one of SEQ ID NOS: 651-699.

In various embodiments, a complementarity determining region of the EGFR binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence set forth in any one of SEQ ID NOS: 700-748.

In various embodiments, a complementarity determining region of the EGFR binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence set forth in any one of SEQ ID NOS: 749-797.

In some embodiments, any of the foregoing EGFR binding domains (e.g., anti-EGFR single domain antibodies of SEQ ID NOS: 798-846) are affinity peptide tagged for ease of purification. In some embodiments, the affinity peptide tag is six consecutive histidine residues, also referred to as 6X-His (SEQ ID NO: 3503).

In certain embodiments, the EGFR binding domains of the present disclosure preferentially bind membrane bound EGFR over soluble EGFR Membrane bound EGFR refers to the presence of EGFR in or on the cell membrane surface of a cell that expresses EGFR. Soluble EGFR refers to EGFR that is no longer on in or on the cell membrane surface of a cell that expresses or expressed EGFR. In certain instances, the soluble EGFR is present in the blood and/or lymphatic circulation in a subject. In one embodiment, the EGFR binding domains bind membrane-bound EGFR at least 5 fold, 10 fold, 15 fold, 20 fold, 25 fold, 30 fold, 40 fold, 50 fold, 100 fold, 500 fold, or 1000 fold greater than soluble EGFR. In one embodiment, the EGFR targeting immune cell engaging proteins of the present disclosure preferentially bind membrane-bound EGFR 30 fold greater than soluble EGFR. Determining the preferential binding of an antigen binding protein to membrane bound EGFR over soluble EGFR can be readily determined using binding assays. It is contemplated that in some embodiments the EGFR binding protein is fairly small and no more than 25 kDa, no more than 20 kDa, no more than 15 kDa, or no more than 10 kDa in some embodiments. In certain instances, the EGFR binding protein is 5 kDa or less if it is a peptide or small molecule entity.

In other embodiments, the EGFR binding proteins described herein comprise small molecule entity (SME) binders for EGFR. SME binders are small molecules averaging about 500 to 2000 Da in size and are attached to the EGFR binding proteins by known methods, such as sortase ligation or conjugation. In these instances, the EGFR binding protein comprises a domain comprising a sortase recognition sequence, e.g., LPETG (SEQ ID NO: 3200). To attach a SME binder to EGFR binding protein comprising a sortase recognition sequence, the protein is incubated with a sortase and a SME binder whereby the sortase attaches the SME binder to the recognition sequence. In yet other embodiments, the EGFR binding proteins described herein comprise a knottin peptide for binding EGFR. Knottins are disulfide-stabilized peptides with a cysteine knot scaffold and have average sizes about 3.5 kDa. Knottins have been contemplated for binding to certain tumor molecules such as EGFR. In further embodiments, the EGFR binding proteins described herein comprise a natural EGFR ligand.

In some embodiments, the EGFR binding protein comprises more than one domain and are of a single-polypeptide design with flexible linkage of the domains. This allows for facile production and manufacturing of the EGFR binding proteins as they can be encoded by single cDNA molecule to be easily incorporated into a vector. Further, in some embodiments where the EGFR binding proteins described herein are a monomeric single polypeptide chain, there are no chain pairing issues or a requirement for dimerization. It is contemplated that, in such embodiments, the EGFR binding proteins described herein have a reduced tendency to aggregate.

In some cases, where the EGFR binding protein comprises more than one domain, the domains within the EGFR binding proteins are conjugated using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See, e.g., Pavel Strp, Veracity of microbial transglutaminase, Bioconjugate Chem. 25, 5, 855-862).

In some examples are provided EGFR binding proteins comprising more than one domain, wherein one of the domains comprises an acceptor glutamine in a constant region, which can then be conjugated to another domain via a lysine-based linker (e.g., any primary amine chain which is a substrate for TGase, e.g. comprising an alkylamine, oxoamine) wherein the conjugation occurs exclusively on one or more acceptor glutamine residues present in the targeting moiety outside of the antigen combining site (e.g., outside a variable region, in a constant region). Conjugation thus does not occur on a glutamine, e.g., an at least partly surface exposed glutamine, within the variable region. The EGFR binding protein, in some examples, is formed by reacting one of the domains with a lysine-based linker in the presence of a TGase.

In some embodiments, where one or more domains within the EGFR binding proteins are directly joined, a hybrid vector is made where the DNA encoding the directly joined domains are themselves directly ligated to each other. In some embodiments, where linkers are used, a hybrid vector is made where the DNA encoding one domain is ligated to the DNA encoding one end of a linker moiety and the DNA encoding another domain is ligated to the other end of the linker moiety.

In some embodiments, the EGFR binding proteins as set forth above are fused to an Fc region from any species, including but not limited to, human immunoglobulin, such as human IgG1, a human IgG2, a human IgG3, human IgG4, to generate Fc-fusion proteins. In some embodiments, the Fc-fusion proteins of this disclosure have extended half-life compared to an otherwise identical EGFR binding protein. In some embodiments, the Fc-fusion EGFR binding proteins of this disclosure contain inter alia one or more additional amino acid residue substitutions, mutations and/or modifications, e.g., in the Fc region, which result in a binding protein with preferred characteristics including, but not limited to: altered pharmacokinetics, extended serum half-life.

In some embodiments, such Fc-fused EGFR binding proteins provide extended half-lives in a mammal, such as in a human, of greater than 5 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-life, in some cases, results in a higher serum titer which thus reduces the frequency of the administration of the EGFR binding proteins and/or reduces the concentration of the antibodies to be administered. Binding to human FcRn in vivo and serum half-life of human FcRn high affinity binding polypeptides is assayed, in some examples, in transgenic mice or transfected human cell lines expressing human FcRn, or in primates to which the polypeptides with a variant Fc region are administered.

The EGFR binding proteins, in some cases, are differentially modified during or after production, e.g., by glycosylation, acetylation, phosphorylation, amidation, derivatization by known protecting/blocking groups, proteolytic cleavage, linkage to an antibody molecule or other cellular ligand, etc. Any of numerous chemical modifications are carried out by techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

Various post-translational modifications of the EGFR binding proteins also encompassed by the disclosure include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. Moreover, the EGFR binding proteins are, in some cases, modified with a detectable label, such as an enzymatic, fluorescent, radioisotopic or affinity label to allow for detection and isolation of the modulator.

In some embodiments, the EGFR binding proteins of the disclosure are monovalent or multivalent bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen).

### FLT3 Binding Proteins

Described herein are immune cell engaging proteins that comprise an FLT3 binding domain as the target antigen binding domain, pharmaceutical compositions thereof, as well as nucleic acids, recombinant expression vectors and host cells for making such proteins thereof. Also provided are methods of using the disclosed proteins comprising an FLT3 binding domain of this disclosure, in the prevention, and/or treatment of diseases, conditions and disorders.

FLT3, also known as fetal liver kinase 2 (FLK-2), stem cell tyrosine kinase 1 (STK-1) and CD135, is a member of the class III receptor tyrosine kinases. Normally, FLT3 is expressed on immature myeloid-lymphocytic precursor cells and dendritic cell precursors, but rarely on mature adult cells. FLT3 is overexpressed in approximately 90% of acute myeloid leukemia (AML), a majority of acute lymphocytic leukemia (ALL) and the blast-crisis phase of chronic myeloid leukemia (BC-CML). Stimulation by FLT3 ligand (FL) enhances the proliferation and survival of leukemia cells. Inhibition of FLT3 signaling leads to apoptosis in dendritic cells and inhibition of immune responses. The MAPK, P13K and Stat5 pathways have been identified to be involved in the downstream signaling of activated FLT3 (See e.g., Stirewalt D L and J P, Radich, J P. Nat Rev Cancer 3:650-665 (2003)).

Described herein are immune cell engaging proteins that comprise an FLT3 binding domain, pharmaceutical compositions thereof, as well as nucleic acids, recombinant expression vectors and host cells for making such proteins thereof. Also provided are methods of using the disclosed proteins comprising an FLT3 binding domain of this disclosure, in the prevention, and/or treatment of diseases, conditions and disorders. In some embodiments, an FLT3 binding domain of this disclosure inhibits FL-induced phosphorylation of wild-type FLT3 and downstream kinases of MPK, P13K, and STAT5 pathways in a disease such as leukemia. In some embodiments, an FLT3 binding domain of this disclosure has an improved ability to activate downstream immune effector functions such as antibody dependent cellular cytotoxicity (ADCC).

In some embodiments, the FLT3 binding domain binds to a human FLT3 protein comprising a sequence as set forth in SEQ ID NO: 3215 (UniProt ID: P36888). In some embodiments, the FLT3 binding domain binds to a protein comprising a truncated sequence compared to SEQ ID NO: 3215 (UniProt ID: P36888).

In some embodiments, the FLT3 binding domains disclosed herein recognize full-length FLT3 (e.g., an FLT3 protein comprising the sequence of SEQ ID NO: 3215 (UniProt ID: P36888). In certain instances, the FLT3 binding domains disclosed herein recognize an epitope within FLT3, such as, in some cases the FLT3 binding proteins interact with one or more amino acids found within a domain of human FLT3. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within a domain of FLT3 (e.g., an FLT3 protein comprising the sequence of SEQ ID NO: 3215 (UniProt ID: P36888). Alternatively, the epitope may consist of a plurality of non-contiguous amino acids (or amino acid sequences) located within a domain of FLT3 (e.g., an FLT3 protein comprising the sequence of SEQ ID NO: 3215 (UniProt ID: P36888).

In some embodiments, the FLT3 binding proteins of this disclosure binds to the full length of an FLT3 protein or to a fragment thereof, such as epitope containing fragments within the full length FLT3 protein, as described above. In some cases, the epitope containing fragment comprises antigenic or immunogenic fragments and derivatives thereof of the FLT3 protein. Epitope containing fragments, including antigenic or immunogenic fragments, are, in some embodiments, 12 amino acids or more, e.g., 20 amino acids or more, 50 or 100 amino acids or more. The FLT3 fragments, in some embodiments, comprises 95% or more of the length of the full protein, 90% or more, 75% or 50% or 25% or 10% or more of the length of the full protein. In some embodiments, the epitope-containing fragments of FLT3 including antigenic or immunogenic fragments are capable of eliciting a relevant immune response in a patient. Derivatives of FLT3 include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the FLT3 sequence (e.g., an FLT3 protein comprising the sequence of SEQ ID NO: 3215 (UniProt ID: P36888).

In some embodiments, the affinity matured FLT3 binding molecule comprises an equivalent or better affinity to a FLT3 protein (such as human FLT3 protein) as that of a FLT3 binding parental molecule, but that has reduced cross reactivity, or in some embodiments, increased cross reactivity, with selected substances, such as ligands, proteins, antigens, or the like, other than the FLT3 epitope for which the FLT3 binding parental molecule is specific, or is designed to be specific for. In regard to the latter, an affinity matured FLT3 binding molecule, in some embodiments, is more successfully tested in animal models if the affinity matured FLT3 binding molecule is reacted with both human FLT3 and the corresponding target of the animal model, e.g. mouse FLT3 or cynomolgus FLT3.

In another example of a substitution to create a variant anti-FLT3 antibody or antigen binding fragments thereof, one or more hypervariable region residues of a parent antibody or antigen binding fragments thereof are substituted. In general, variants are then selected based on improvements in desired properties compared to a parent antibody, for example, increased affinity, reduced affinity, reduced immunogenicity, increased pH dependence of binding.

In some embodiments, the FLT3 binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the FLT3 binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues.

In some embodiments, the binding proteins described herein comprise a polypeptide having a sequence selected from SEQ ID NOS: 1004-1079 and 3495-3496, subsequences thereof, and variants thereof. In some embodiments, the FLT3 binding protein comprises at least 75%-95% or more homology to a sequence selected from SEQ ID NOS: 1004-1079 and 3495-3496, subsequences thereof, and variants thereof. In some embodiments, the FLT3 binding protein comprises at least 75%, 80%, 85%, 90%, 95%, 96%, 97%, 98%, 99%, or more identity to a sequence selected from SEQ ID NOS: 1004-1079 and 3495-3496, subsequences thereof, and variants thereof. In some embodiments, the FLT3 binding protein comprises at least 60%-95% or more identity to a sequence selected from SEQ ID NOS: 1004-1079 and 3495-3496, subsequences thereof, and variants thereof. In some embodiments, the FLT3 binding protein comprises at least 75%, 80%, 85%, 90%, 95%, 96%, 97%, 98%, 99%, or more identity to a sequence selected from SEQ ID NOS: 1004-1079, subsequences thereof, and variants thereof.

In some embodiments, the CDR1 comprises the amino acid sequence selected from the group consisting of SEQ ID NOS: 1080-1155, and 3497-3498, or a sequence comprising one or more amino acid substitutions in a sequence selected from the group consisting of SEQ ID NOS: 1080-1155, and 3497-3498. In some embodiments, the CDR2 comprises the amino acid sequence selected from the group consisting of SEQ ID NOS: 1156-1231, and 3499-3500 or a sequence comprising one or more amino acid substitutions in a sequence selected from the group consisting of SEQ ID NOS: 1156-1231, and 3499-3500. In some embodiments, the CDR3 comprises the amino acid sequence selected from the group consisting of SEQ ID NOS: 1232-1307, and 3501-3502 or a sequence comprising one or more amino acid substitutions in a sequence selected from the group consisting of SEQ ID NOS: 1232-1307, and 3501-3502. In some embodiments, the CDR1 comprises the amino acid sequence selected from the group consisting of SEQ ID NOS: 1150, 1152, 3497, and 3498, or a sequence comprising one or more amino acid substitutions in a sequence selected from the group consisting of SEQ ID NOS: 1150, 1152, 3497, and 3498. In some embodiments, the CDR2 comprises the amino acid sequence selected from the group consisting of SEQ ID NOS: 1226, 1228, 3499, and 3500, or a sequence comprising one or more amino acid substitutions in a sequence selected from the group consisting of SEQ ID NOS: 1226, 1228, 3499, and 3500. In some embodiments, the CDR3 comprises the amino acid sequence selected from the group consisting of SEQ ID NOS: 1302, 1304, 3501, and 3502 or a sequence comprising one or more amino acid substitutions in a sequence selected from the group consisting of SEQ ID NOS: 1293 or 1302.

In various embodiments, the FLT3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 1004-1079, and 3495-3496.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 1080-1155, and 3497-3498.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 1156-1231 and 3499-3500.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 1232-1307 and 3501-3502.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 1074, 1076, 3495, and 3496 and wherein the FLT3 binding domain comprises a humanized FLT3 binding domain.

In some embodiments, any of the foregoing FLT3 binding domains (e.g., anti-FLT3 single domain antibodies of SEQ ID NOS: 1004-1079) are affinity peptide tagged for ease of purification. In some embodiments, the affinity peptide tag is six consecutive histidine residues, also referred to as 6X-his (SEQ ID NO: 3503). In certain embodiments, the FLT3 binding domains of the present disclosure preferentially bind membrane bound FLT3 over soluble FLT3 Membrane bound FLT3 refers to the presence of FLT3 in or on the cell membrane surface of a cell that expresses FLT3. Soluble FLT3 refers to FLT3 that is no longer on in or on the cell membrane surface of a cell that expresses or expressed FLT3. In certain instances, the soluble FLT3 is present in the blood and/or lymphatic circulation in a subject. In one embodiment, the FLT3 binding domains bind membrane-bound FLT3 at least 5 fold, 10 fold, 15 fold, 20 fold, 25 fold, 30 fold, 40 fold, 50 fold, 100 fold, 500 fold, or 1000 fold greater than soluble FLT3. In one embodiment, the FLT3 binding proteins of the present disclosure preferentially bind membrane-bound FLT3 30 fold greater than soluble FLT3. Determining the preferential binding of an antigen binding protein to membrane bound FLT3 over soluble FLT3 can be readily determined using binding assays.

It is contemplated that in some embodiments the FLT3 binding protein is fairly small and no more than 25 kDa, no more than 20 kDa, no more than 15 kDa, or no more than 10 kDa in some embodiments. In certain instances, the FLT3 binding protein is 5 kDa or less if it is a peptide or small molecule entity.

In other embodiments, the FLT3 binding proteins described herein comprise small molecule entity (SME) binders for FLT3. SME binders are small molecules averaging about 500 to 2000 Da in size and are attached to the FLT3 binding proteins by known methods, such as sortase ligation or conjugation. In these instances, the FLT3 binding protein comprises a domain comprising a sortase recognition sequence, e.g. LPETG (SEQ ID NO: 3200). To attach a SME binder to FLT3 binding protein comprising a sortase recognition sequence, the protein is incubated with a sortase and a SME binder whereby the sortase attaches the SME binder to the recognition sequence. In yet other embodiments, the FLT3 binding proteins described herein comprise a knottin peptide for binding FLT3. Knottins are disulfide-stabilized peptides with a cysteine knot scaffold and have average sizes about 3.5 kDa. Knottins have been contemplated for binding to certain tumor molecules such as FLT3. In further embodiments, the FLT3 binding proteins described herein comprise a natural FLT3 ligand.

In some embodiments, the FLT3 binding protein comprises more than one domain and are of a single-polypeptide design with flexible linkage of the domains. This allows for facile production and manufacturing of the FLT3 binding proteins as they can be encoded by single cDNA molecule to be easily incorporated into a vector. Further, in some embodiments where the FLT3 binding proteins described herein are a monomeric single polypeptide chain, there are no chain pairing issues or a requirement for dimerization. It is contemplated that, in such embodiments, the FLT3 binding proteins described herein have a reduced tendency to aggregate.

In some cases, where the FLT3 binding protein comprises more than one domain, the domains within the FLT3 binding proteins are conjugated using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See e.g., Pavel Strp, Veracity of microbial transglutaminase, Bioconjugate Chem. 25, 5, 855-862.

In some examples are provided FLT3 binding proteins comprising more than one domain, wherein one of the domains comprises an acceptor glutamine in a constant region, which can then be conjugated to another domain via a lysine-based linker (e.g. any primary amine chain which is a substrate for TGase, e.g. comprising an alkylamine, oxoamine) wherein the conjugation occurs exclusively on one or more acceptor glutamine residues present in the targeting moiety outside of the antigen combining site (e.g., outside a variable region, in a constant region). Conjugation thus does not occur on a glutamine, e.g. an at least partly surface exposed glutamine, within the variable region. The FLT3 binding protein, in some examples, is formed by reacting one of the domains with a lysine-based linker in the presence of a TGase.

In some embodiments, where one or more domains within the FLT3 binding proteins are directly joined, a hybrid vector is made where the DNA encoding the directly joined domains are themselves directly ligated to each other. In some embodiments, where linkers are used, a hybrid vector is made where the DNA encoding one domain is ligated to the DNA encoding one end of a linker moiety and the DNA encoding another domain is ligated to the other end of the linker moiety.

In certain embodiments, the FLT3 binding proteins according to the present disclosure may be incorporated into immune cell engaging proteins. In some embodiments, the immune cell engaging proteins comprise a CD3 binding domain, a half-life extension domain, and an FLT3 binding domain according to this disclosure. In some embodiments, the FLT3 binding trispecific protein comprises a trispecific antibody.

### EpCAM Binding Proteins

Described herein are immune cell engaging proteins that bind EpCAM, pharmaceutical compositions thereof, as well as nucleic acids, recombinant expression vectors and host cells for making such proteins thereof. Also provided are methods of using the disclosed EpCAM binding proteins in the prevention, and/or treatment of diseases, conditions and disorders. In some embodiments, the EpCAM binding proteins are part of immune cell engaging proteins that comprise an EpCAM binding domain as described herein.

The epithelial cell adhesion molecule (EpCAM) is a membrane glycoprotein that is expressed in most normal human epithelia and overexpressed in most carcinomas. This molecule is responsible for cell-to-cell adhesion and additionally participates in signaling, cell migration, proliferation and differentiation. Therefore, EpCAM has been the target of immunotherapy in clinical trials of several solid tumors. It has been found to play an important role in the detection and isolation of circulating tumor cells (CTCs). EpCAM has been shown in various studies to be beneficial in diagnosis and therapy of various carcinomas. Furthermore, in many cases, tumor cells were observed to express EpCAM to a much higher degree than their parental epithelium or less aggressive forms of said cancers. For example, EpCAM expression was shown to be significantly higher on neoplastic tissue and in adenocarcinoma than on normal prostate epithelium (n=76; p<0.0001), suggesting that increased EpCAM expression represents an early event in the development of prostate cancer. Seem Poczatek, J Urol., 1999, 162, 1462-1644. In addition, in the majority of both squamous and adenocarcinomas of the cervix a strong EpCAM expression has been shown to correlate with an increased proliferation and the disappearance of markers for terminal differentiation. See Litvinov, Am. J Pathol. 1996, 148, 865-75. One example is breast cancer where overexpression of EpCAM on tumor cells is a predictor of survival. See Gastl, Lancet. 2000, 356, 1981-1982. Furthermore, EpCAM has been described as a marker for the detection of disseminated tumor cells in patients suffering from squamous cell carcinoma of the head, neck and lung. See Chaubal, Anticancer Res 1999, 19, 2237-2242, Piyathilake, Hum Pathol. 2000, 31, 482-487. Normal squamous epithelium, as found in epidermis, oral cavity, epiglottis, pharynx, larynx and esophagus did not significantly express EpCAM. See Quak, Hybridoma, 1990, 9, 377-387.

EpCAM is contemplated to serve to adhere epithelial cells in an oriented and highly ordered fashion. See Litvinov, J Cell Biol. 1997, 139, 1337-1348. Upon malignant transformation of epithelial cells the rapidly growing tumor cells are believed to abandon the high cellular order of epithelia. Consequently, the surface distribution of EpCAM is contemplated to become less restricted and the molecule better exposed on tumor cells. Due to their epithelial cell origin, tumor cells from most carcinomas are expected to express EpCAM on their surface.

EpCAM, is a 40-kDa membrane-integrated glycoprotein of 314 amino acids with specific expression in certain epithelia and on many human carcinomas. See, e.g., in Balzar, J. Mol. Med. 1999, 77, 699-712). EpCAM was discovered and subsequently cloned through its recognition by the murine monoclonal antibody 17-1A/edrecolomab. See Goettlinger, Int J Cancer. 1986; 38, 47-53 and Simon, Proc. Natl. Acad. Sci. USA. 1990; 87, 2755-2759. Monoclonal antibody 17-1A was generated by immunization of mice with human colon carcinoma cells. See Koprowski, Somatic Cell Genet. 1979, 5, 957-971. The EGF-like repeats of EpCAM were shown to mediate lateral and reciprocal interactions in homophilic cell adhesion. See, e.g., Balzar, Mol. Cell. Biol. 2001, 21, 2570-2580) and, for that reason, is predominantly located between epithelial cells (Litvinov, J Cell Biol. 1997, 139, 1337-1348, Balzar, J Mol Med. 1999, 77, 699-712 and Trebak, J Biol Chem. 2001, 276, 2299-2309).

EpCAM is also known by the following alternate names: Epithelial Cell Adhesion Molecule, Tumor-Associated Calcium Signal Transducer, Major Gastrointestinal Tumor-Associated Protein GA733-2, Adenocarcinoma-Associated Antigen, Cell Surface Glycoprotein Trop-1, Epithelial Glycoprotein 314, TACSTD1, EGP314, MIC18, TROP1, M4S1, KSA, Membrane Component Chromosome 4 Surface marker (35 kD glycoprotein), Antigen identified by monoclonal antibody AUA-1, human epithelial glycoprotein-2, epithelial cell surface antigen, epithelial glycoprotein, KS 1/4Antigen, CD326 Antigen, GA722-2, HEGP314, HNPCC8, Ep-CAM, DIAR5, EGP-2, EGP40, KS ¼, MK-1, M1S2, ESA, and EGP. Exemplary protein sequences for EpCAM is provided in UniProtkB ID NOS: P16422 and B5MCA4. In some embodiments, the EpCAM binding proteins of this disclosure binds to an EpCAM sequence provided in UniProtkB ID NOS: P16422 (SEQ ID NO: 478) or B5MCA4 (SEQ ID NO: 475).

In some embodiments, the EpCAM binding domain binds to an extracellular domain of the mature EpCAM protein. The human extracellular domain sequence is provided in SEQ ID NO: 3212; the cynomolgus extracellular domain sequence is provided in SEQ ID NO: 3213; and the mouse extracellular domain sequence is provided in SEQ ID NO: 3214.

In some embodiments, the EpCAM binding domain binds to a protein comprising a truncated sequence compared to SEQ ID NO: 3208. In some embodiments, the EPCAM binding domain binds to a protein comprising the sequence of SEQ ID NO: 3208. In some embodiments, the EpCAM binding domain binds to a protein comprising a truncated sequence compared to SEQ ID NO: 3209. In some embodiments, the EpCAM binding domain binds to a protein comprising the sequence of SEQ ID NO: 3209. In some embodiments, the EpCAM binding domain binds to a protein comprising a truncated sequence compared to SEQ ID NO: 3210. In some embodiments, the EpCAM binding domain binds to a protein comprising a truncated sequence compared to SEQ ID NO: 3210. In some embodiments, the EpCAM binding domain binds to a protein comprising a truncated sequence compared to SEQ ID NO: 478. In some embodiments, the EpCAM binding domain binds to a protein comprising a truncated sequence compared to SEQ ID NO: 3211. In some embodiments, the EpCAM binding domain binds to a protein comprising a truncated sequence compared to SEQ ID NO: 3212. In some embodiments, the EpCAM binding domain binds to a protein comprising a truncated sequence compared to SEQ ID NO: 3212. In some embodiments, the EpCAM binding domain binds to a protein comprising a truncated sequence compared to SEQ ID NO: 3213. In some embodiments, the EpCAM binding domain binds to a protein comprising a truncated sequence compared to SEQ ID NO: 3213. In some embodiments, the EpCAM binding domain binds to a protein comprising a truncated sequence compared to SEQ ID NO: 3214. In some embodiments, the EpCAM binding domain binds to a protein comprising a truncated sequence compared to SEQ ID NO: 3214.

In some embodiments, the EpCAM binding domains disclosed herein recognize full-length EpCAM. In certain instances, the EpCAM binding domains disclosed herein recognize an epitope within EpCAM, such as, in some cases the EpCAM binding proteins interact with one or more amino acids found within a domain of human EpCAM. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within a domain of EpCAM. Alternatively, the epitope may consist of a plurality of non-contiguous amino acids (or amino acid sequences) located within a domain of EpCAM.

In some embodiments, the EpCAM binding proteins of this disclosure binds to the full length EpCAM protein or to a fragment thereof, such as epitope containing fragments within the full length EpCAM protein, as described above. In some cases, the epitope containing fragment comprises antigenic or immunogenic fragments and derivatives thereof of the EpCAM protein. Epitope containing fragments, including antigenic or immunogenic fragments, are, in some embodiments, 12 amino acids or more, e.g., 20 amino acids or more, 50 or 100 amino acids or more. The EpCAM fragments, in some embodiments, comprises 95% or more of the length of the full protein, 90% or more, 75% or 50% or 25% or 10% or more of the length of the full protein. In some embodiments, the epitope-containing fragments of EpCAM including antigenic or immunogenic fragments are capable of eliciting a relevant immune response in a patient. Derivatives of EpCAM include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the EpCAM sequence provided in SEQ ID NOS: 3208-3214.

In another example of a substitution to create a variant anti-EpCAM antibody or antigen binding fragments thereof, one or more hypervariable region residues of a parent antibody are substituted. In general, variants are then selected based on improvements in desired properties compared to a parent antibody or antigen binding fragments thereof, for example, increased affinity, reduced affinity, reduced immunogenicity, increased pH dependence of binding.

In some embodiments, the EpCAM binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the EpCAM binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the EpCAM binding domain comprises an amino acid sequence selected from SEQ ID NOS: 961-1003.

In some embodiments, the EpCAM binding protein comprises at least 70%, 75%, 80%, 85%, 90%, 95%, 96%, 97%, 98%, 99%, or more identity to a sequence selected from SEQ ID NOS: 961-1003, subsequences thereof, and variants thereof. In some embodiments, the EpCAM binding protein comprises at least 70%-95% or more identity to a sequence selected from SEQ ID NOS: 961-1003, subsequences thereof, and variants thereof.

In some embodiments, the CDR1 comprises the amino acid sequence as set forth in any one of SEQ ID NOS: 847-884 or a sequence comprising one or more substitutions compared to a sequence selected from the group consisting of SEQ ID NOS: 847-884. In some embodiments, the CDR2 comprises a sequence as set forth in any one of SEQ ID Nos. 885-922 or a sequence comprising one or more substitutions compared to a sequence selected from the group consisting of SEQ ID NOS: 885-922. In some embodiments, the CDR3 comprises a sequence as set forth in any one of SEQ ID NOS: 923-960 a sequence comprising one or more substitutions compared to a sequence selected from the group consisting of SEQ ID NOS: 923-960.

In various embodiments, a complementarity determining region of the EpCAM binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 847-884.

In various embodiments, a complementarity determining region of the EpCAM binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 885-922.

In various embodiments, a complementarity determining region of the EpCAM binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 923-960.

In some embodiments, any of the foregoing EpCAM binding domains (e.g., anti-EpCAM single domain antibodies of SEQ ID NOS: 961-1003) are affinity peptide tagged for ease of purification. In some embodiments, the affinity peptide tag is six consecutive histidine residues, also referred to as 6X-his (SEQ ID NO: 3503).

In certain embodiments, the EpCAM binding domains of the present disclosure preferentially bind membrane bound EpCAM over soluble EpCAM Membrane bound EpCAM refers to the presence of EpCAM in or on the cell membrane surface of a cell that expresses EpCAM. Soluble EpCAM refers to EpCAM that is no longer on in or on the cell membrane surface of a cell that expresses or expressed EpCAM. In certain instances, the soluble EpCAM is present in the blood and/or lymphatic circulation in a subject. In one embodiment, the EpCAM binding domains bind membrane-bound EpCAM at least 5 fold, 10 fold, 15 fold, 20 fold, 25 fold, 30 fold, 40 fold, 50 fold, 100 fold, 500 fold, or 1000 fold greater than soluble EpCAM. In one embodiment, the EpCAM binding proteins of the present disclosure preferentially bind membrane-bound EpCAM 30 fold greater than soluble EpCAM. Determining the preferential binding of an antigen binding protein to membrane bound EpCAM over soluble EpCAM can be readily determined using binding assays.

It is contemplated that in some embodiments the EpCAM binding protein is fairly small and no more than 25 kDa, no more than 20 kDa, no more than 15 kDa, or no more than 10 kDa in some embodiments. In certain instances, the EpCAM binding protein is 5 kDa or less if it is a peptide or small molecule entity.

In other embodiments, the EpCAM binding proteins described herein comprise small molecule entity (SME) binders for EpCAM. SME binders are small molecules averaging about 500 to 2000 Da in size and are attached to the EpCAM binding proteins by known methods, such as sortase ligation or conjugation. In these instances, the EpCAM binding protein comprises a domain comprising a sortase recognition sequence, e.g., LPETG (SEQ ID NO: 3200). To attach a SME binder to EpCAM binding protein comprising a sortase recognition sequence, the protein is incubated with a sortase and a SME binder whereby the sortase attaches the SME binder to the recognition sequence. In yet other embodiments, the EpCAM binding proteins described herein comprise a knottin peptide for binding EpCAM. Knottins are disulfide-stabilized peptides with a cysteine knot scaffold and have average sizes about 3.5 kDa. Knottins have been contemplated for binding to certain tumor molecules such as EpCAM. In further embodiments, the EPCAM binding proteins described herein comprise a natural EpCAM ligand.

In some embodiments, the EpCAM binding protein comprises more than one domain and are of a single-polypeptide design with flexible linkage of the domains. This allows for facile production and manufacturing of the EpCAM binding proteins as they can be encoded by single cDNA molecule to be easily incorporated into a vector. Further, in some embodiments where the EpCAM binding proteins described herein are a monomeric single polypeptide chain, there are no chain pairing issues or a requirement for dimerization. It is contemplated that, in such embodiments, the EpCAM binding proteins described herein have a reduced tendency to aggregate.

In the EpCAM binding proteins comprising more than one domain, the domains are linked by one or more internal linker. In certain embodiments, the internal linkers are “short,” i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, the internal linkers are “long,” i.e., consist of 15, 20 or 25 amino acid residues. In some embodiments, the internal linkers consist of about 3 to about 15, for example 8, 9 or 10 contiguous amino acid residues. Regarding the amino acid composition of the internal linkers, peptides are selected with properties that confer flexibility to the EpCAM binding proteins, do not interfere with the binding domains as well as resist cleavage from proteases. For example, glycine and serine residues generally provide protease resistance. Examples of internal linkers suitable for linking the domains in the EpCAM binding proteins include but are not limited to (GS)ₙ (SEQ ID NO: 3859), (GGS)ₙ (SEQ ID NO: 3860), (GGGS)ₙ (SEQ ID NO: 3861), (GGSG)ₙ (SEQ ID NO: 3862), (GGSGG)ₙ (SEQ ID NO: 3863), (GGGGS)ₙ (SEQ ID NO: 3864), (GGGGG)ₙ (SEQ ID NO: 3865), or (GGG)ₙ (SEQ ID NO: 3866), wherein n is 1, 2, 3, 4, 5, 6, 7, 8, 9, or 10. In one embodiment, the linker is (GGGGSGGGGSGGGGSGGGGS)(SEQ ID NO: 3867), (GGGGSGGGGSGGGGS) (SEQ ID NO: 3868), LPETG (SEQ ID NO: 3869), (GGGGSGGGS) (SEQ ID NO: 3871) or SGGG (SEQ ID NO: 3872).

In some cases, where the EpCAM binding protein comprises more than one domain, the domains within the EpCAM binding proteins are conjugated using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See, e.g., Pavel Strp, Veracity of microbial transglutaminase, Bioconjugate Chem. 25, 5, 855-862.

In some examples are provided EpCAM binding proteins comprising more than one domain, wherein one of the domains comprises an acceptor glutamine in a constant region, which can then be conjugated to another domain via a lysine-based linker (e.g. any primary amine chain which is a substrate for TGase, e.g. comprising an alkylamine, oxoamine) wherein the conjugation occurs exclusively on one or more acceptor glutamine residues present in the targeting moiety outside of the antigen combining site (e.g., outside a variable region, in a constant region). Conjugation thus does not occur on a glutamine, e.g. an at least partly surface exposed glutamine, within the variable region. The EpCAM binding protein, in some examples, is formed by reacting one of the domains with a lysine-based linker in the presence of a TGase.

In some embodiments, where one or more domains within the EpCAM binding proteins are directly joined, a hybrid vector is made where the DNA encoding the directly joined domains are themselves directly ligated to each other. In some embodiments, where linkers are used, a hybrid vector is made where the DNA encoding one domain is ligated to the DNA encoding one end of a linker moiety and the DNA encoding another domain is ligated to the other end of the linker moiety.

In certain embodiments, the EpCAM binding proteins according to the present disclosure may be incorporated into immune cell engaging proteins. In some embodiments, the immune cell engaging proteins comprise a CD3 binding domain, a half-life extension domain, and an EpCAM binding domain according to this disclosure. In some embodiments, the immune cell engaging protein comprises a trispecific antibody.

### CD3 Binding Domain

The immune cell engaging protein described herein comprises an immune cell engaging domain. In some embodiments, the immune cell engaging domain comprises a natural killer (NK) cell engaging domain, a T cell engaging domain, a B cell engaging domain, a dendritic cell engaging domain, a macrophage cell engaging domain, or a combination thereof. In some embodiments, the immune cell engaging protein comprises a T-cell engaging domain. In some embodiments, the T cell engaging domain is a CD3 binding domain.

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the T cell receptor (TCR) as well as and CD3 ζ (zeta) altogether to comprise the T cell receptor complex. Clustering of CD3 on T cells, such as by immobilized anti-CD3 antibodies leads to T cell activation similar to the engagement of the T cell receptor but independent of its clone-typical specificity.

In one aspect, the single chain variable fragment CD3 binding proteins described herein comprise a domain which specifically binds to CD3. In one aspect, the single chain variable fragment CD3 binding proteins described herein comprise a domain which specifically binds to human CD3. In one aspect, the single chain variable fragment CD3 binding proteins described herein comprise a domain which specifically binds to cynomolgus CD3. In one aspect, the single chain variable fragment CD3 binding proteins described herein comprise a domain which binds to human CD3 and cynomolgus CD3. In some embodiments, the single chain variable fragment CD3 binding proteins described herein comprise a domain which specifically binds to CD3γ. In some embodiments, the single chain variable fragment CD3 binding proteins described herein comprise a domain which specifically binds to CD36. In some embodiments, the single chain variable fragment CD3 binding proteins described herein comprise a domain which specifically binds to CD3ε.

In another aspect is provided an immune cell engaging protein comprising a single chain variable fragment CD3 binding protein according to the present disclosure. In some embodiments, the immune cell engaging protein comprising a single chain variable fragment CD3 binding protein according to the present disclosure specifically binds to the T cell receptor (TCR). In certain instances, the immune cell engaging protein comprising a single chain variable fragment CD3 binding protein according to the present disclosure binds the α chain of the TCR. In certain instances, the immune cell engaging protein comprising a single chain variable fragment CD3 binding protein according to the present disclosure binds the β chain of the TCR.

In certain embodiments, the CD3 binding domain of the immune cell engaging protein described herein exhibit not only potent CD3 binding affinities with human CD3, but show also excellent crossreactivity with the respective cynomolgus monkey CD3 proteins. In some instances, the CD3 binding domain of the immune cell engaging protein binding proteins are cross-reactive with CD3 from cynomolgus monkey. In certain instances, the Kd for binding human CD3 (hKd) is about the same as the Kd for binding cynomolgus CD3 (cKd). In certain instances, the ratio between hKd and cKd (hKd:cKd) is between about 20:1 to about 1:2.

In some embodiments, the CD3 binding domain of immune cell engaging protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the immune cell engaging protein will ultimately be used. For example, for use in humans, it may be beneficial for the CD3 binding domain to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (CDR2), and heavy chain complementary determining region 3 (CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (NUVION®), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD3 binding domain is a single chain variable fragment (scFv) comprising a light chain and a heavy chain of an amino acid sequence provided herein. As used herein, “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g. substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following configurations: light chain variable region-scFv linker-heavy chain variable region or heavy chain variable region-scFv linker-light chain variable region.

In some instances, scFvs which bind to CD3 are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the CD3 binding site. In certain embodiments, such scFv linkers are “short”, i.e. consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 10 or 15 contiguous amino acid residues. Regarding the amino acid composition of the scFv linkers, peptides are selected that confer flexibility, do not interfere with the variable domains as well as allow inter-chain folding to bring the two variable domains together to form a functional CD3 binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD3 binding and production yield of the scFv. Examples of peptide scFv linkers suitable for linking a variable light chain domain and a variable heavy chain domain in a scFv include but are not limited to (GS)ₙ (SEQ ID NO: 3859), (GGS)ₙ (SEQ ID NO: 3860), (GGGS)ₙ (SEQ ID NO: 3861), (GGSG)ₙ (SEQ ID NO: 3862), (GGSGG)ₙ (SEQ ID NO: 3863), (GGGGS)ₙ (SEQ ID NO: 3864), (GGGGG)ₙ (SEQ ID NO: 3865), or (GGG)ₙ (SEQ ID NO: 3866), wherein n is 1, 2, 3, 4, 5, 6, 7, 8, 9, or 10. In one embodiment, the linker is (GGGGSGGGGSGGGGSGGGGS)(SEQ ID NO: 3867), (GGGGSGGGGSGGGGS) (SEQ ID NO: 3868), LPETG (SEQ ID NO: 3869), (GGGGSGGGS) (SEQ ID NO: 3871) or SGGG (SEQ ID NO: 3872). Variation in the linker length may retain or enhance activity, giving rise to superior efficacy in activity studies.

In some embodiments, CD3 binding domain of an immune cell engaging protein has an affinity to CD3 on CD3 expressing cells with a K_{d} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of a single chain variable fragment CD3 binding protein has an affinity to CD3ε, γ, or δ with a K_{d} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, CD3 binding domain of a single chain variable fragment CD3 binding protein has low affinity to CD3, i.e., about 100 nM or greater.

In certain embodiments, the single chain variable fragment CD3 binding proteins described herein bind to human CD3 with a human Kd (hKd) and to cynomolgus CD3 with a cyno Kd (cKd). In some embodiments, hKd and cKd are between about between about 1 nM to about 2 nM, about 3 nM to about 5 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM.

In some embodiments, the hKd and cKd of the single chain variable fragment CD3 binding proteins is about the same as the Kd of a CD3 binding protein having the sequence as set forth is SEQ ID NO: 3167. In some embodiments, the hKd and cKd of the single chain variable fragment CD3 binding proteins is about 1.1 fold to about 1.5 fold the Kd of a CD3 binding protein having the sequence as set forth is SEQ ID NO: 3167. In some embodiments, the hKd and cKd of the single chain variable fragment CD3 binding proteins is about 1.5 fold to about 2 fold the Kd of a CD3 binding protein having the sequence as set forth is SEQ ID NO: 3167. In some embodiments, the hKd and cKd of the single chain variable fragment CD3 binding proteins is about 2.5 fold to about 3 fold the Kd of a CD3 binding protein having the sequence as set forth is SEQ ID NO: 3167. In some embodiments, the hKd and cKd of the single chain variable fragment CD3 binding proteins is about 3 fold to about 5 fold the Kd of a CD3 binding protein having the sequence as set forth is SEQ ID NO: 3167. In some embodiments, the hKd and cKd of the single chain variable fragment CD3 binding proteins is about 6 fold to about 15 fold the Kd of a CD3 binding protein having the sequence as set forth is SEQ ID NO: 3167. In some embodiments, the hKd and cKd of the single chain variable fragment CD3 binding proteins is about 15 fold to about 20 fold the Kd of a CD3 binding protein having the sequence as set forth is SEQ ID NO: 3167. In some embodiments, the hKd and cKd of the single chain variable fragment CD3 binding proteins is about 20 fold to about 50 fold the Kd of a CD3 binding protein having the sequence as set forth is SEQ ID NO: 3167. In some embodiments, the hKd and cKd of the single chain variable fragment CD3 binding proteins is about 55 fold to about 70 fold the Kd of a CD3 binding protein having the sequence as set forth is SEQ ID NO: 3167. In some embodiments, the hKd and cKd of the single chain variable fragment CD3 binding proteins is about 75 fold to about 100 fold the Kd of a CD3 binding protein having the sequence as set forth is SEQ ID NO: 3167. In some embodiments, the hKd and cKd of the single chain variable fragment CD3 binding proteins is about 120 fold to about 200 fold the Kd of a CD3 binding protein having the sequence as set forth is SEQ ID NO: 3167.

In some embodiments, the ratio between the hKd and cKd (hKd: cKd) ranges from about 20:1 to about 1:2. The affinity to bind to CD3 can be determined, for example, by the ability of the single chain variable fragment CD3 binding protein itself or its CD3 binding domain to bind to CD3 coated on an assay plate; displayed on a microbial cell surface; in solution; etc. The binding activity of the single chain variable fragment CD3 binding protein itself or its CD3 binding domain of the present disclosure to CD3 can be assayed by immobilizing the ligand (e.g., CD3) or the single chain variable fragment CD3 binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high or low pH, and the like and analyzed, for example, by Surface Plasmon Resonance (SPR).

In some embodiments, the single chain variable fragment CD3 binding protein has an amino acid sequence selected from SEQ ID NOS: 3153-3169. In various embodiments, the single chain variable fragment CD3 binding protein comprises an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 3153-3169.

### Single Domain Serum Albumin Binding Protein

The immune cell engaging protein described herein is a half-life extended protein. In some embodiments, the immune cell engaging protein comprises a domain binds to serum albumin. In some embodiments, the serum albumin is human serum albumin (HSA).

Serum albumin is produced by the liver, occurs dissolved in blood plasma and is the most abundant blood protein in mammals. Albumin is essential for maintaining the oncotic pressure needed for proper distribution of body fluids between blood vessels and body tissues; without albumin, the high pressure in the blood vessels would force more fluids out into the tissues. It also acts as a plasma carrier by non-specifically binding several hydrophobic steroid hormones and as a transport protein for hemin and fatty acids. Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma. In some embodiments, the single domain serum albumin binding proteins bind to HSA. In some embodiments, the single domain serum albumin binding proteins bind to serum albumin protein from cynomolgus monkeys. In some embodiments, the single domain serum albumin binding proteins bind to HSA and serum albumin protein from cynomolgus monkeys. In some embodiments, the single domain serum albumin binding proteins also bind to mouse serum albumin protein. In some embodiments, the binding affinity towards mouse serum albumin is about 1.5-fold to about 20-fold weaker than that towards human or cynomolgus serum albumin.

Noncovalent association with albumin extends the elimination half-time of short-lived proteins. For example, a recombinant fusion of an albumin binding domain to a Fab fragment resulted in a decrease in in vivo clearance by 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action/serum half-life.

In some embodiments, the single-domain serum albumin binding proteins described herein is a single domain antibody such as a heavy chain variable domain (VH), a variable domain (VHH) of camelid derived sdAb, peptide, ligand or small molecule entity specific for serum albumin. In some embodiments, the single-domain serum albumin binding proteins described herein is a single domain antibody such as a heavy chain variable domain (VH), a variable domain (VHH) of camelid derived sdAb, peptide, ligand or small molecule entity specific for HSA. In some embodiments, the serum albumin binding domain of a single domain serum albumin binding protein described herein is any domain that binds to serum albumin including but not limited to domains from a monoclonal antibody, a polyclonal antibody, a recombinant antibody, a human antibody, a humanized antibody. In certain embodiments, the serum albumin binding domain is a single-domain antibody. In other embodiments, the serum albumin binding domain is a peptide. In further embodiments, the serum albumin binding domain is a small molecule. It is contemplated that the single domain serum albumin binding protein is fairly small and no more than 25 kD, no more than 20 kD, no more than 15 kD, or no more than 10 kD in some embodiments. In certain instances, the single domain serum albumin binding protein binding is 5 kD or less if it is a peptide or small molecule entity.

In some embodiments, the single domain serum albumin binding protein described herein is a half-life extension domain which provides for altered pharmacodynamics and pharmacokinetics of the single domain serum albumin binding protein itself. As above, the half-life extension domain extends the elimination half-time. The half-life extension domain also alters pharmacodynamic properties including alteration of tissue distribution, penetration, and diffusion of the single domain serum albumin binding protein. In some embodiments, the half-life extension domain provides for improved tissue (including tumor) targeting, tissue distribution, tissue penetration, diffusion within the tissue, and enhanced efficacy as compared with a protein without a half-life extension domain. In one embodiment, therapeutic methods effectively and efficiently utilize a reduced amount of the single domain serum albumin binding protein, resulting in reduced side effects, such as reduced non-tumor cell cytotoxicity.

Further, the binding affinity of the single domain serum albumin binding protein towards its binding target can be selected so as to target a specific elimination half-time in a particular single domain serum albumin binding protein. Thus, in some embodiments, the single domain serum albumin binding protein has a high binding affinity towards its binding target. In other embodiments, the single domain serum albumin binding protein has a medium binding affinity towards its binding target. In yet other embodiments, the single domain serum albumin binding protein has a low or marginal binding affinity towards its binding target. Exemplary binding affinities include K_{D} of 10 nM or less (high), between 10 nM and 100 nM (medium), and greater than 100 nM (low). As above, binding affinities of the single domain serum albumin binding proteins towards binding targets are determined by known methods such as Surface Plasmon Resonance (SPR).

In some embodiments, the single domain serum albumin binding protein has an amino acid sequence selected from SEQ ID NOS: 3185-3193. In various embodiments, the single chain variable fragment CD3 binding protein comprises an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 3185-3193.

In some embodiments, the single domain serum albumin binding protein has an elimination half-time of at least 1 hour, at least 2 hours, at least 4 hours, at least 6 hours, at least 12 hours, at least 20 hours, at least 25 hours, at least 30 hours, at least 35 hours, at least 40 hours, at least 45 hours, at least 50 hours, or at least 100 hours.

### Immune Cell Engaging Protein Modifications

The immune cell engaging protein described herein, including antigen binding domains and immune cell engaging domains encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the immune cell engaging protein described herein, including the peptide backbone, the amino acid side-chains, and the amino or carboxyl termini. Certain common peptide modifications that are useful for modification of the FLT3 binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, derivatives of the immune cell engaging protein as described herein comprise immunoreactive modulator derivatives and antigen binding molecules comprising one or more modifications.

In some embodiments, the immune cell engaging protein of the disclosure are monovalent or multivalent bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen).

In some embodiments, the immune cell engaging protein as set forth above are fused to an Fc region from any species, including but not limited to, human immunoglobulin, such as human IgG1, a human IgG2, a human IgG3, human IgG4, to generate Fc-fusion FLT3 binding proteins. In some embodiments, the Fc-fusion immune cell engaging protein of this disclosure have extended half-life compared to an otherwise identical immune cell engaging protein. In some embodiments, the Fc-fusion immune cell engaging protein of this disclosure contain inter alia one or more additional amino acid residue substitutions, mutations and/or modifications, e.g., in the Fc region, which result in a binding protein with preferred characteristics including, but not limited to: altered pharmacokinetics, extended serum half-life.

In some embodiments, such Fc-fused immune cell engaging protein provide extended half-lives in a mammal, such as in a human, of greater than 5 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-life, in some cases, results in a higher serum titer which thus reduces the frequency of the administration of the immune cell engaging protein and/or reduces the concentration of the antibodies to be administered. Binding to human FcRn in vivo and serum half-life of human FcRn high affinity binding polypeptides is assayed, in some examples, in transgenic mice or transfected human cell lines expressing human FcRn, or in primates to which the polypeptides with a variant Fc region are administered.

The immune cell engaging protein, in some cases, are differentially modified during or after production, e.g., by glycosylation, acetylation, phosphorylation, amidation, derivatization by known protecting/blocking groups, proteolytic cleavage, linkage to an antibody molecule or other cellular ligand, etc. Any of numerous chemical modifications are carried out by techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

Various post-translational modifications of the immune cell engaging protein also encompassed by the disclosure include, for example, N-linked or O-linked carbohydrate chains, processing of N-terminal or C-terminal ends, attachment of chemical moieties to the amino acid backbone, chemical modifications of N-linked or 0-linked carbohydrate chains, and addition or deletion of an N-terminal methionine residue as a result of prokaryotic host cell expression. Moreover, the FLT3 binding proteins are, in some cases, modified with a detectable label, such as an enzymatic, fluorescent, radioisotopic or affinity label to allow for detection and isolation of the modulator.

### Peptide Mask

The extended-release binding protein described herein, in some embodiments, comprises a masking peptide. When bound to the antigen binding domain(s) of the immune cell engaging protein, the masking peptide, in some embodiments, blocks, occludes, inhibits (e.g., decreases) or otherwise prevents (e.g., masks) the activity or binding of the antigen binding domain(s) to the target(s). In some embodiments, the masking peptide interferes with the binding of the antigen binding domain to a target antigen. In some embodiments, the immune cell engaging protein comprises a CD3 binding domain and the masking peptide is specific for the CD3 binding domain and interferes with binding of the CD3 binding domain to its target.

The masking peptide, in certain instances, is covalently linked to the N-terminus or C-terminus of immune cell engaging protein, for example, through a cleavable linker. In some embodiments, the masking peptide comprises a sequence selected from the group consisting of SEQ ID NOS: 3663-3682, or a sequence comprising one or more amino acid substitutions relative to a sequence selected from the group consisting of SEQ ID NOS: 3663-3682.

In some embodiments, the masking peptide is a peptide that is 5 amino acids in length, 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, or longer. In some embodiments, the masking peptide is at least 3 amino acids in length. In some embodiments, the masking peptide is 3 to 12 amino acids, 4 to 15 amino acids, 5 to 20 amino acids, or 6 to 25 amino acids, in length. In some embodiments, the masking peptide is 6 amino acids in length. In some embodiments, the masking peptide is a linear peptide or a cyclic peptide. In some embodiments, the cyclized peptide is formed by a di-sulfide bond connecting two cysteine amino acid residues. In some embodiments, the cysteine amino acid residues are terminal cysteines, located at or near the N-terminus and/or the C-terminus of the masking peptide. In some embodiments, the di-sulfide bond connects an N-terminal cysteine with a C-terminal cysteine.

In some embodiments, the masking peptide comprises a sequence selected from the group consisting of SEQ ID NOS: 3663-3682, or a sequence comprising one or more amino acid substitutions (e.g., two or three amino acid substitutions) relative to a sequence selected from the group consisting of SEQ ID NOS: 3663-3682.

### Cleavable Linker

As described above, in some embodiments, the masking peptide and the half-life extended immune cell engaging protein are connected through a cleavable linker. The cleavable linker, in some embodiments, facilitates release of an active immune cell engaging protein in a cell. Examples of cleavable linker include but are not limited to: an acid-labile linker, a peptidase-sensitive linker, a photolabile linker, a dimethyl linker or disulfide-containing linker (see, e.g., Chari et al., Cancer Res. 52:127-131 (1992); U.S. Pat. No. 5,208,020). The cleavable linker, in some embodiments, comprises a sequence that is recognized by a protease. Examples of proteases include, but are not limited to: ABHD12, ADAM12, ABHD12B, ABHD13, ABHD17A, ADAM 19, ADAM20, ADAM21, ADAM28, ADAM30, ADAM33, ADAM8, ABHD17A, ADAMDEC1, AD AMTS 1, AD AMTS 10, AD AMTS 12, AD AMTS 13, AD AMTS 14, AD AMTS 15, AD AMTS 16, AD AMTS 17, AD AMTS 18, AD AMTS 19, ADAMTS2, ADAMTS20, AD AMTS 3, AD AMTS 4, ABHD17B, AD AMTS 5, AD AMTS 6, ADAMTS 7, ADAMTS 8, ADAMTS 9, ADAMTSL1, ADAMTSL2, ADAMTSL3, ABHD17C, ADAMTSL5, ASTL, BMP1, CELA1, CELA2A, CELA2B, CELA3A, CELA3B, ADAM 10, ADAM 15, ADAM 17, ADAM9, ADAMTS4, CTSE, CTSF, ADAMTSL4, CMA1, CTRB 1, CTRC, CTSO, CTR1, CTSA, CTSW, CTSB, CTSC, CTSD, ESP1, CTSG, CTSH, GZMA, GZMB, GZMH, CTSK, GZMM, CTSL, CTSS, CTSV, CTSZ, HTRA4, KLK10, KLK11, KLK13, KLK14, KLK2, KLK4, DPP4, KLK6, KLK7, KLKB 1, ECE1, ECE2, ECEL1, MASP2, MEP1A, MEP1B, ELANE, FAP, GZMA, MMP11, GZMK, HGFAC, HPN, HTRA1, MMP11, MMP 16, MMP17, MMP 19, HTRA2, MMP20, MMP21, HTRA3, HTRA4, KEL, MMP23B, MMP24, MMP25, MMP26, MMP27, MMP28, KLK5, MMP3, MMP7, MMP8, MMP9, LGMN, LNPEP, MASP1, PAPPA, PAPPA2, PCSK1, NAPSA, PCSK5, PCSK6, MME, MMP1, MMP10, PLAT, PLAU, PLG, PRSS 1, PRSS 12, PRSS2, PRSS21, PRSS3, PRSS33, PRSS4, PRSS55, PRSS57, MMP 12, PRSS8, PRSS9, PRTN3, MMP13, MMP14, ST14, TMPRSS10, TMPRSS11A, TMPRSS11D, TMPRSS11E, TMPRSS11F, TMPRSS12, TMPRSS13, MMP15, TMPRSS15, MMP2, TMPRSS2, TMPRSS3, TMPRSS4, TMPRSS5, TMPRSS6, TMPRSS7, TMPRSS9, NRDC, OVCH1, PAMR1, PCSK3, PHEX, TINAG, TPSAB1, TPSD1, and TPSGE In some embodiments, the cleavable peptide is cleaved by one or more enzyme selected from the group consisting of: ADAM 17, HTRA1, PRSS1, FAP, GZMK, NAPSA, MMP1, MMP2, MMP9, MMP10, MMP7, MMP12, MMP28, AD AMTS 9, HGFAC, and HTRA3.

Exemplary protease recognition sequences are provided Table 1:

| SEQ | | |
|---|---|---|
| ID | | |
| No. | Description | Sequence |
| 3716 | protease cleavage site | VANLA |
| 3717 | protease cleavage site | PLGL |
| 3718 | protease cleavage site | PQAS |
| 3719 | protease cleavage site | PKPQA |
| 3720 | protease cleavage site | PTGLE |
| 3721 | protease cleavage site | PENFF |
| 3722 | protease cleavage site | AATLK |
| 3723 | protease cleavage site | PQDLL |
| 3724 | protease cleavage site | PAGL |
| 3725 | protease cleavage site | VLGL |
| 3726 | protease cleavage site | RLQLKL |
| 3727 | protease cleavage site | RTRYED |
| 3728 | protease cleavage site | KPLGL |
| 3729 | Protease: MMP7 | KRALGLPG |
| 3730 | Protease: MMP7 | (DE)8RPLALWRS(DR)8 |
| | Protease: MMP9 | PR(S/T)(L/I)(S/T) |
| 3732 | Protease: MMP9 | LEATA |
| 3733 | Protease: MMP11 | GGAANLVRGG |
| 3734 | Protease: MMP14 | SGRIGFLRTA |
| 3735 | Protease: MMP | PLGLAG |
| 3736 | Protease: MMP | PLGLAX |
| 3737 | Protease: MMP | PLGC(me)AG |
| 3738 | Protease: MMP | ESPAYYTA |
| 3739 | Protease: MMP | RLQLKL |
| 3740 | Protease: MMP | RLQLKAC |
| 3741 | Protease: MMP2, MMP9, | EP(Cit)G(Hof)YL |
| | MMP14 | |
| 3742 | Protease: Urokinase | SGRSA |
| | plasminogen | |
| | activator (uPA) | |
| 3743 | Protease: Urokinase | DAFK |
| | plasminogen | |
| | activator (uPA) | |
| 3744 | Protease: Urokinase | GGGRR |
| | plasminogen | |
| | activator (uPA) | |
| 3745 | Protease: Lysosomal Enzyme | GFLG |
| 3746 | Protease: Lysosomal Enzyme | ALAL |
| | Protease: Lysosomal Enzyme | FK |
| | Protease: Cathepsin B | NLL |
| 3749 | Protease: Cathepsin D | PIC(Et)FF |
| 3750 | Protease: Cathepsin K | GGPRGLPG |
| 3751 | Protease: Prostate | HSSKLQ |
| | Specific Antigen | |
| 3752 | Protease: Prostate | HSSKLQL |
| | Specific Antigen | |
| 3753 | Protease: Prostate | HSSKLQEDA |
| | Specific Antigen | |
| 3754 | Protease: Herpes Simplex | LVLASSSFGY |
| | Virus Protease | |
| 3755 | Protease: HIV Protease | GVSQNYPIVG |
| 3756 | Protease: CMV Protease | GVVQASCRLA |
| | Protease: Thrombin | F(Pip)RS |
| 3758 | Protease: Thrombin | DPRSFL |
| 3759 | Protease: Thrombin | PPRSFL |
| 3760 | Protease: Caspase-3 | DEVD |
| 3761 | Protease: Caspase-3 | DEVDP |
| 3762 | Protease: Caspase-3 | KGSGDVEG |
| 3763 | Protease: Interleukin | GWEHDG |
| | 1β converting | |
| | enzyme | |
| 3764 | Protease: Enterokinase | EDDDDKA |
| 3765 | Protease: FAP | KQEQNPGST |
| 3766 | Protease: Kallikrein 2 | GKAFRR |
| 3767 | Protease: Plasmin | DAFK |
| 3768 | Protease: Plasmin | DVLK |
| 3769 | Protease: Plasmin | DAFK |
| 3770 | Protease: TOP | ALLLALL |

In some embodiments, a linker as described herein comprises a sequence selected from the sequences provided in Table 2. In some embodiments, a linker as described herein further comprises sequences flanked on the N-terminal and/or C-terminal. In some embodiments, the flanked sequences may be (but are not limited to) the following: GGGG (SEQ ID NO: 3883), GGGS (SEQ ID NO: 3884), GGGT (SEQ ID NO: 3885), GGGGG (SEQ ID NO: 3886), GGGGS (SEQ ID NO: 3887), and/or GGGGT (SEQ ID NO: 3888).

TABLE 2
Selected linker sequences

| SEQ ID | | |
|---|---|---|
| NO: | Description | Sequence |
| 3683 | non-cleavable | SGGGGSGGVV |
| 3684 | non-cleavable | SGGGGSGGGGSGGGGS |
| 3685 | non-cleavable | SGGGGSGGGGSGGGGGS |
| 3686 | non-cleavable | SGGGGSGGGS |
| 3687 | non-cleavable | GGGGSGGGGSGGGGSGGG |
| 3688 | L001 | KPLGLQARVV |
| 3689 | L040 | PQASTGRSGG |
| 3690 | L041 | PQGSTGRAAG |
| 3691 | L042 | PPASSGRAGG |
| 3692 | L043 | PQGSTARSAG |
| 3693 | L045 | PIPVQGRAH |
| 3694 | L054 | VANLASTGRA |
| 3695 | L055 | PLGLSTGRA |
| 3696 | L056 | PQASTGRA |
| 3697 | L057 | PKPQASTGRA |
| 3698 | L058 | PTGLESTGRA |
| 3699 | L059 | PENFFSTGRA |
| 3700 | L060 | AATLKSTGRA |
| 3701 | L061 | PQDLLSTGRA |
| 3702 | L062 | PAGLSTGRA |
| 3703 | L063 | VLGLSTGRA |
| 3704 | L064 | VANLASTGA |
| 3705 | L065 | PLGLSTGA |
| 3706 | L066 | PQASTGA |
| 3707 | L067 | PKPQASTGA |
| 3708 | L068 | PTGLESTGA |
| 3709 | L069 | PENFFSTGA |
| 3710 | L070 | AATLKSTGA |
| 3711 | L071 | PQDLLSTGA |
| 3712 | L072 | PAGLSTGA |
| 3713 | L073 | VLGLSTGA |
| 3714 | L075 | RTRYEDGGS |
| 3715 | L076 | PQASTGGSG |
| 3878 | L085 | KPLGLQAGVV |

### Polynucleotides Encoding the Extended-Release Binding Protein

Also provided, in some embodiments, are polynucleotide molecules encoding the extended-release binding protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the immune cell engaging protein or gene encoding various domains of the immune cell engaging protein comprising more than one domain. In some embodiments, the gene encoding the domains are either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide coding for an extended-release binding protein as described herein is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described immune cell engaging protein. Examples of expression vectors for expression in E. coli are pSKK (Le Gall et al., J Immunol Methods. (2004) 285(1):111-27) or pcDNA5 (Invitrogen) for expression in mammalian cells.

Thus, the extended-release binding protein as described herein, in some embodiments, are produced by introducing a vector encoding the protein as described above into a host cell and culturing said host cell under conditions whereby the protein domains are expressed, may be isolated and, optionally, further purified.

Protein with Masking Peptide

Described herein is a pharmaceutical composition comprising a protein which comprises a binding moiety, a masking peptide, and a cleavable linker. Without being bound by any theory, it is contemplated that the protein with the masking peptide described herein is able to reduce target-mediated drug disposition when administered to a subject by gradually releasing the active form of the binding moiety when it is in systemic circulation.

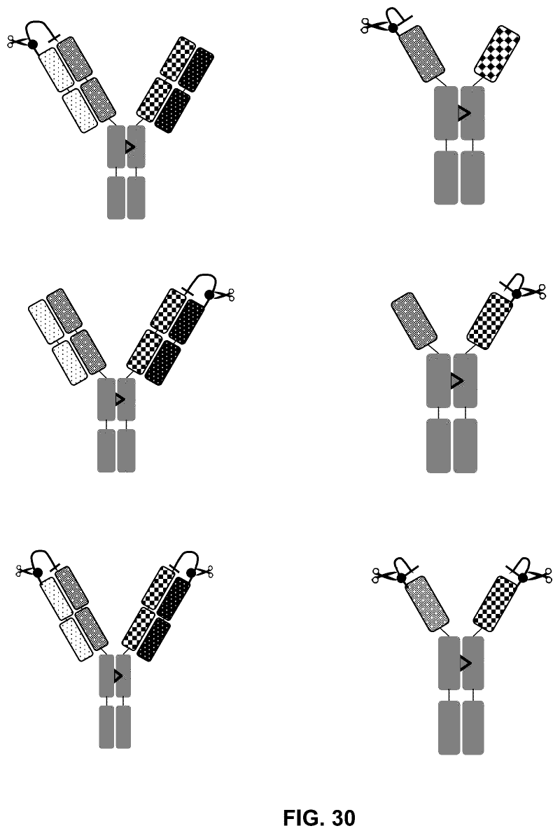

In some aspects, the cleavable linker is significantly cleaved in systemic circulation. In some aspects, the half-life of the protein described herein in systemic circulation is longer than a comparable protein without the masking peptide. In some aspects, a comparable protein without the masking peptide has a nonlinear pharmacokinetics (PK) across a dose range evaluated, and the protein described herein has an improved linearity of PK across the dose range evaluated compared to the comparable protein. In some aspects, the molar amount of the protein's binding moiety bound to the target when administered to the subject is lower compared to the molar amount of a binding moiety of a comparable protein without the masking peptide when administered to the subject at a same dose level. In some aspects, the binding rate between the protein's binding moiety and the target when administered to the subject is lower compared to the binding rate between a binding moiety of a comparable protein without the masking peptide and the target. FIG. 30 provides exemplary constructs of the protein with masking peptide described herein.

### Targets

The binding moiety of the protein with masking peptide descried herein may bind to various targets. A non-exhaustive list of the targets is ICOS (inducible T cell co-stimulator, CD278), OX40 (CD134, TNFRSF4, tumor necrosis factor receptor superfamily member 4), CD40 (TNFRSF5, tumor necrosis factor receptor superfamily member 5), DR5 (death receptor 5, TRAIL receptor 2), GITR (glucocorticoid-induced TNFR-related protein, TNFRSF18, tumor necrosis factor receptor superfamily member 18), and 4-1BB (CD137, TNFRSF9, tumor necrosis factor receptor superfamily member 9).

In some embodiments, the target may be ICOS. ICOS is a T-cell specific, CD28-superfamily costimulatory molecule and immune checkpoint protein. ICOS is normally expressed on certain activated T cells and plays a key role in the proliferation and activation of T cells.

In some embodiments, the target may be OX40. OX40 is a cell surface glycoprotein and member of the tumor necrosis factor receptor superfamily (TNFRSF). OX40 is expressed on T lymphocytes and plays an essential role in T-cell activation. Co-stimulation of activated T cells with agonistic monoclonal antibodies (mAb) against the tumor necrosis factor receptor superfamily member OX40 offers a novel immunotherapeutic approach to cancer. OX40 engagement may co-stimulate effector T cells and deplete regulatory T cells, resulting in enhanced tumor immunity.

In some embodiments, the target may be CD40. CD40 is a stimulatory receptor and a member of the tumor necrosis factor (TNF) receptor superfamily. CD40 is expressed on various immune cells, such as macrophages, dendritic cells and various tumor cell types, such as many B-cell malignancies, and some solid tumors. CD40 plays a key role in the activation of the immune system, mediates both indirect tumor cell killing through the activation of the immune system and direct tumor cell apoptosis. CD40 is highly expressed on most B-lineage hematologic malignancies including multiple myeloma, non-Hodgkin lymphoma, chronic lymphocytic leukemia, Hodgkin disease and acute lymphoblastic leukemia.

In some embodiments, the target may be DR5. DR5, also known as TRAIL receptor 2 (TRAILR2) and tumor necrosis factor receptor superfamily member 10B (TNFRSF10B), is a cell surface receptor of the TNF-receptor superfamily that binds TRAIL and mediates apoptosis. DR5 contains an intracellular death domain. DR5 can be activated by tumor necrosis factor-related apoptosis inducing ligand (TNFSF10/TRAIL/APO-2L), and transduces apoptosis signal. TRAIL, a member of the TNF superfamily of cytokines, plays a key role in the induction of apoptosis through TRAIL-mediated death receptor pathways.

In some embodiments, the target may be GITR (glucocorticoid-induced tumor necrosis factor receptor; tumor necrosis factor superfamily, member 18; TNFRSF18). GITR is a TNF receptor superfamily costimulatory molecule expressed primarily by regulatory T cells (Treg), effector T cells, and natural killer cells that inhibits the suppressive activity of Tregs. Agonistic antibodies or GITR ligand binding to GITR in concert with T cell receptor (TCR) stimulation causes activation of the MAPK/ERK pathway and NFkB, resulting in augmentation of T cell proliferation and proinflammatory cytokine production and enhanced anti-tumor effector function, as well as resistance of CD4+ and CD8+ T cells to Treg suppression. In tumor models, signaling through GITR has been shown to inhibit Treg proliferation, induce Treg depletion, and cause tumor regression.

In some embodiments, the target may be 4-1BB. 4-1BB is a member of the tumor necrosis factor (TNF)/nerve growth factor (NGF) family of receptors and is expressed by activated T- and B-lymphocytes and monocytes. 4-1BB's ligand has been found to play an important role in the regulation of immune responses.

### Antibodies

The protein with the masking peptide described herein may include an antibody. A non-exhaustive list of the antibody is GSK3359609 (GSK609, feladilimab), PF-8600 (PF-04518600, ivuxolimab), JNJ-64457107 (JNJ-107, JNJ 7107, ADC-1013, mitazalimab), CP-870,893, SGN-40 (huS2C6, Dacetuzumab), MEDI3039, ABBV-621 (eftozanermin alfa, APG880), MED11873 (efgivanermin alfa), AMG 228, PF-05082566 (Utomilumab, uto), and urelumab (BMS-663513).

In some embodiments, the antibody may be GSK3359609 (GSK609, feladilimab). GSK3359609 is an agonistic antibody for the inducible T-cell co-stimulator (ICOS; CD278), with potential immune checkpoint inhibitory and antineoplastic activities. Upon administration, GSK3359609 targets and binds to ICOS expressed on tumor infiltrating CD4-positive T cells. This stimulates ICOS-positive T-cell proliferation, enhances cytotoxic T-lymphocyte (CTL) survival and increases CTL-mediated immune responses against tumor cells.

In some embodiments, the antibody may be PF-8600 (PF-04518600, ivuxolimab). PF-8600 is a fully human agonist IgG2 mAb that targets the co-stimulatory receptor OX40 (CD134; TNFRSF4), with potential immunostimulatory activity. Upon administration, PF-8600 selectively binds to and activates OX40; which induces proliferation of memory and effector T lymphocytes. In the presence of tumor-associated antigens (TAAs), this may promote a T-cell-mediated immune response against TAA-expressing tumor cells.

In some embodiments, the antibody may be JNJ-64457107 (JNJ-107, JNJ 7107, ADC-1013, mitazalimab). JNJ-64457107 is a human immunoglobulin (Ig) G1 monoclonal antibody directed against the cell surface receptor CD40 with potential immunostimulatory and antineoplastic activities. Upon intratumoral administration, JNJ-64457107 binds to CD40 on antigen-presenting dendritic cells, which leads to the activation and proliferation of effector and memory T cells, and enhances the immune response against tumor cells. In addition, this agent binds to the CD40 antigen present on the surfaces of tumor cells, which induces antibody-dependent cytotoxicity (ADCC). This eventually inhibits the proliferation of CD40-expressing tumor cells.

In some embodiments, the antibody may be CP-870,893. CP-870,893 is a fully human monoclonal antibody (mAb) agonist of the cell surface receptor CD40 with potential immunostimulatory and antineoplastic activities. Similar to the CD40 ligand (CD40L or CD154), CP-870,893 binds to CD40 on a variety of immune cell types, triggering the cellular proliferation and activation of antigen-presenting cells (APCs), activating B cells and T cells, and enhancing the immune response; in addition, this agent may activate CD40 present on the surfaces of some solid tumor cells, resulting in apoptosis and decreased tumor growth.

In some embodiments, the antibody may be SGN-40 (huS2C6, Dacetuzumab). SGN-40 is a humanized monoclonal antibody directed against the CD40 receptor with potential antineoplastic activity. SGN-40 specifically binds to and inhibits the CD40 receptor, thereby inducing apoptosis and inhibiting cellular proliferation via antibody-dependent cellular cytotoxicity (ADCC) in cells that overexpress this receptor.

In some embodiments, the antibody may be MEDI3039. MEDI3039 is a highly potent multivalent DR5 agonist. MEDI3039 is a modified protein derived from the third fibronectin type III domain of the glycoprotein tenascin C, which possesses a region similar to the variable region characteristic of antibodies. An optimized multivalent DR5 agonist was highly potent in triggering cell death in multiple TRAIL-sensitive cell lines, was one to two orders of magnitude more potent than TRAIL, and showed promising results in multiple cancer cells (colon, lung, leukemia, liver cancers) and in vivo colon cancer models.

In some embodiments, the antibody may be ABBV-621 (eftozanermin alfa, APG880). ABBV-621 is a fusion protein composed of a tumor necrosis factor (TNF)-related apoptosis-inducing ligand (TRAIL) receptor agonist consisting of six receptor binding domains (RBDs) of TRAIL fused to the Fc-domain of a human immunoglobulin G1 (IgG1) antibody, with potential pro-apoptotic and antineoplastic activities. Upon administration, ABBV-621 binds to TRAIL-receptors, pro-apoptotic death receptors (DRs) TRAIL-R1 (death receptor 4; DR4) and TRAIL-R2 (death receptor 5; DR5), expressed on tumor cells, thereby inducing tumor cell apoptosis. ABBV-621 is designed to maximize receptor clustering for optimal efficacy.

In some embodiments, the antibody may be MEDI1873 (efgivanermin alfa). MEDI1873 is a homogenous hexameric agonist fusion protein composed of the extracellular domain (ECD) of the T-cell costimulatory receptor human GITR ligand (GITRL) and an immunoglobulin (Ig) G1 Fc domain, with potential immunomodulating and antineoplastic activities. Upon administration, MEDI1873 binds to and activates GITR found on multiple types of T cells, thereby inducing both the activation and proliferation of tumor antigen-specific T-effector cells. This abrogates the suppression of T-effector cells which is induced by inappropriately activated T-regulatory cells (Tregs), suppresses Tregs and decreases Treg tumor infiltration, and activates the immune system to help eradicate tumor cells.

In some embodiments, the antibody may be AMG 228. AMG 228 is an agonistic human IgG1 monoclonal antibody that binds to human GITR similar to MEDI1873.

In some embodiments, the antibody may be PF-05082566 (Utomilumab, uto). PF-05082566 is a humanized agonist IgG2 monoclonal antibodies for the tumor necrosis factor superfamily receptors 4-1BB. PF-05082566's binding to human 4-1BB results in NF-κB activation and downstream cytokine production in cell lines and primary lymphocytes. PF-05082566 can induce human leukocyte proliferation and has demonstrated significant antitumor activity as a single agent in human peripheral blood lymphocyte (PBL) SCID xenograft tumor models.

In some embodiments, the antibody may be urelumab. Urelumab is a humanized agonistic monoclonal antibody targeting the 4-1BB receptor with potential immunostimulatory and antineoplastic activities. Urelumab specifically binds to and activates 4-1BB-expressing immune cells, stimulating an immune response, in particular a cytotoxic T cell response, against tumor cells.

### Methods of Treatment

Also provided herein, in some embodiments, are methods and uses for stimulating the immune system of an individual in need thereof comprising administration of an extended-release binding protein and the protein with masking peptide as described herein. In some instances, administration induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue. In some embodiments, the target antigen is an immune checkpoint protein.

Also provided herein are methods and uses for a treatment of a disease, disorder or condition associated with a target antigen comprising administering to an individual in need thereof an extended-release binding protein and the protein with masking peptide as described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one embodiment, the cancer is a hematological cancer. In one embodiment, the cancer is a melanoma. In one embodiment, the cancer is lung cancer. In one embodiment, the cancer is ovarian cancer. In one embodiment, the cancer is prostate cancer. In one embodiment, the cancer is pancreatic cancer. In one embodiment, the cancer is mesothelioma. In one embodiment, the cancer is neuroendocrine cancers. In one embodiment, the cancer is multiple myeloma. In one embodiment, the cancer is breast cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the extended-release binding proteins and the protein with masking peptide described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the extended-release binding proteins described herein are administered in combination with anti-diarrheal agents, anti-emetic agents, analgesics, opioids and/or non-steroidal anti-inflammatory agents. In some embodiments, the conditionally active binding proteins described herein is administered before, during, or after surgery.

Administration of the extended-release binding protein and the protein with masking peptide described herein can be effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. In some embodiments, the extended-release binding protein is injected or injectable.

### EXAMPLES

The examples below further illustrate the described embodiments without limiting the scope of the disclosure.

### Example 1: Exemplary Constructs of an Extended-Release Binding Protein

An exemplary construct of an extended-release binding protein is shown in FIG. 1. TriTAC-XR is a tri-specific binding protein with anti-target, anti-albumin and anti-CD3 binding capabilities. The intact form of the extended-release binding protein (FIG. 1A) comprises a CD3 mask linked to the binding protein with a cleavable linker. When the cleavable linker is gradually cleaved in the subject, it releases the active version of the extended-release binding protein (FIG. 1B). FIG. 1C shows the predicted concentrations of the intact version and the active version in systemic circulation after multiple administration events. FIG. 1D shows the predicted concentration of the active version of a corresponding antigen binding protein without the masking peptide in systemic circulation after multiple administration events.

Additional extended-release binding protein constructs are shown in FIGS. 2A-L with various configurations of the anti-target, anti-albumin and anti-CD3 binding domains. FIGS. 2A-C and FIGS. 2G-I illustrate the intact versions and FIGS. 2D-F and FIGS. 2J-L illustrate the active versions after cleavage of the linker.

### Example 2: Production and Purification of the Extended-Release Binding Proteins Used in the Present Study

Protein Production: Sequences of exemplary TriTAC-XR molecules were cloned into mammalian expression vector pcDNA3.4 (Invitrogen) preceded by a leader sequence and followed by a 6× histidine tag (SEQ ID NO: 3870). Expi293F cells (Life Technologies A14527) were maintained in suspension in between 0.2 to 8×10⁶ cells/ml in Expi293 media. Purified plasmid DNA was transfected into Expi293 cells in accordance with Expi293 Expression System Kit (Life Technologies, A14635) protocols, and maintained for 4-6 days post transfection. The amount of the exemplary TriTAC-XR proteins in conditioned media was quantitated using an Octet RED96 instrument with Protein A tips (ForteBio/Sartorius) using a control trispecific protein for a standard curve.

Protein Purification: Conditioned media from either host cell was filtered and partially purified by affinity and desalting chromatography. TriTAC-XR proteins were subsequently polished by ion exchange and upon fraction pooling formulated in a neutral buffer containing excipients. Final purity was assessed by SDS-PAGE and analytical SEC using an Acquity BEH SEC 200 1.7u 4.6×150 mm column (Waters Corporation) resolved in an aqueous/organic mobile phase with excipients at neutral pH on a 1290 LC system and peaks integrated with Chemstation CDS software (Agilent).

### Example 3: PSMA Targeting Extended-Release Binding Proteins Induced T Cell-Dependent Cellular Cytotoxicity Assay

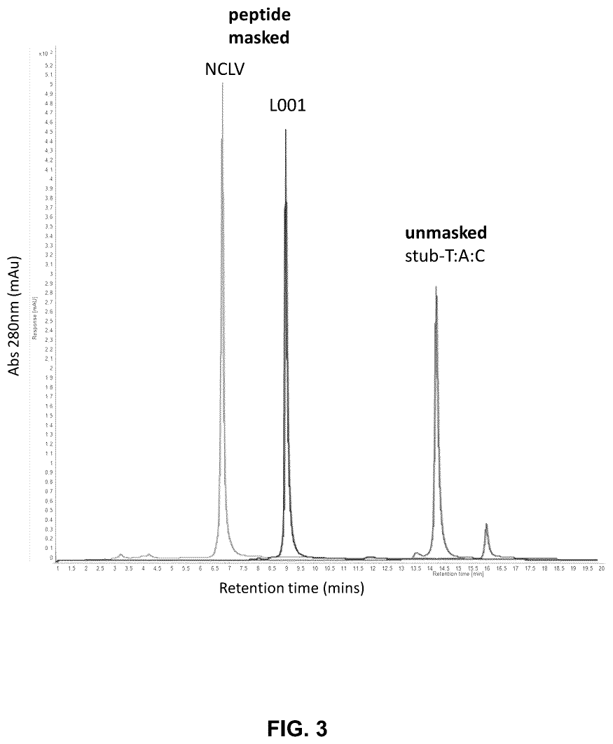

The binding proteins used in this study are summarized in Table 3. Stub:T:A:C is a PSMA binding protein without the masking domain. As described herein, stub refers to the residual linker remaining on the active drug fragment after protease cleavage. Peptide:NCLV is a PSMA binding protein with a peptide mask and non-cleavable linker. Peptide:L001 is a PSMA targeting extended-release binding protein with a peptide mask and linker L001, which is a cleavable linker. Ion exchange chromatography profiles (FIG. 3) demonstrate the purity of the PSMA targeting proteins and also show that there is little or no active drug present (stub:T:A:C) in the purified peptide:L001 protein.

TABLE 3
PSMA Binding Proteins and EC₅₀ values

| | | | Fold over |
|---|---|---|---|
| Protein | SEQ ID NO | EC₅₀ (pM) | stub:T:A:C |
| stub:T:A:C | 3609 | 5 | 1 |
| peptide:NCLV | 3601 | 6256 | 1249 |
| peptide:L001 | 3600 | 81 | 16 |

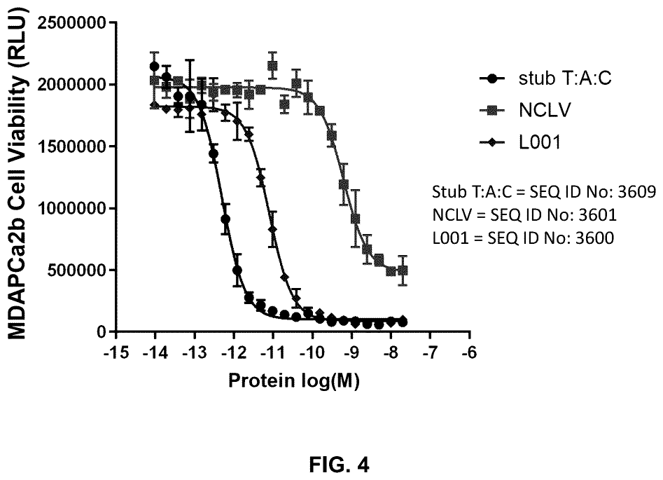

TDCC Assay: For functional potency in a T cell-dependent cellular cytotoxicity assays, soluble test molecules shown in Table 3 were incubated in multi-well plates, with purified resting human T cells (effector cell) and MDAPCa2b cancer cells at 10:1 effector: target cell ratio for 48 h at 37° C. in the presence of 15 mg/ml human serum albumin (HSA). The MDAPCa2b cells had been stably transduced with a luciferase reporter gene to allow specific T cell-mediated cell killing measurement by Steady-Glo (Promega). Results shown in FIG. 4 demonstrates slow activation of peptide masked PSMA targeting extended-release binding proteins in TDCC assay. The EC₅₀ values are provided in Table 3. The efficiency of masking is quantitated by the fold increase in EC₅₀ of peptide:NCLV (SEQ ID NO: 3601) or peptide:L001 (SEQ ID NO: 3600) over stub:T:A:C (SEQ ID NO: 3609) (Table 3).

### Example 4: PSMA Targeting Extended-Release Binding Proteins with Different Peptide Mask Sequences Induced T Cell-Dependent Cellular Cytotoxicity Assay

The binding proteins with various masking peptides used in this study are summarized in Table 4. The configuration of the anti-target, anti-albumin and anti-CD3 binding domains are according to FIG. 2A (SEQ ID NOS: 3601 to 3608) and FIG. 2D (SEQ ID NO: 3609) except SEQ ID NO: 3610, which is a ProTriTAC molecule. Description a ProTriTAC molecule's structure can be found in WO2019222283A1, which is incorporated herein by reference.

TABLE 4
PSMA Binding Proteins and EC₅₀ values

| | | | Fold |
|---|---|---|---|
| | | | in- |
| | | | crease |
| | | | in |
| | | | EC₅₀ |
| SEQ | | | over |
| ID | | EC₅₀ | SEQ ID |
| NO | Protein | (M) | NO 3609 |
| 3601 | QDGNEE:NCLV:αPSMA:αALB:αCD3 | 1E−09 | 720 |
| 3602 | LDGNEE:NCLV:αPSMA:αALB:αCD3 | 7E−12 | 5 |
| 3603 | PDGNEE:NCLV:αPSMA:αALB:αCD3 | 7E−12 | 5 |
| 3604 | DDGNEE:NCLV:αPSMA:αALB:αCD3 | 3E−12 | 2 |
| 3605 | EDGNEE:NCLV:αPSMA:αALB:αCD3 | 8E−12 | 5 |
| 3606 | QEGNEE:NCLV:αPSMA:αALB:αCD3 | 2E−10 | 160 |
| 3607 | QDANEE:NCLV:αPSMA:αALB:αCD3 | 2E−10 | 130 |
| 3608 | QDSNEE:NCLV:αPSMA:αALB:αCD3 | 1E−11 | 8 |
| 3609 | stub:αPSMA:αALB:αCD3 | 1E−12 | 1 |
| 3610 | αALB-MASK027::NCLV:αPSMA: | 2E−10 | 170 |
| | αALB:αCD3 | | |

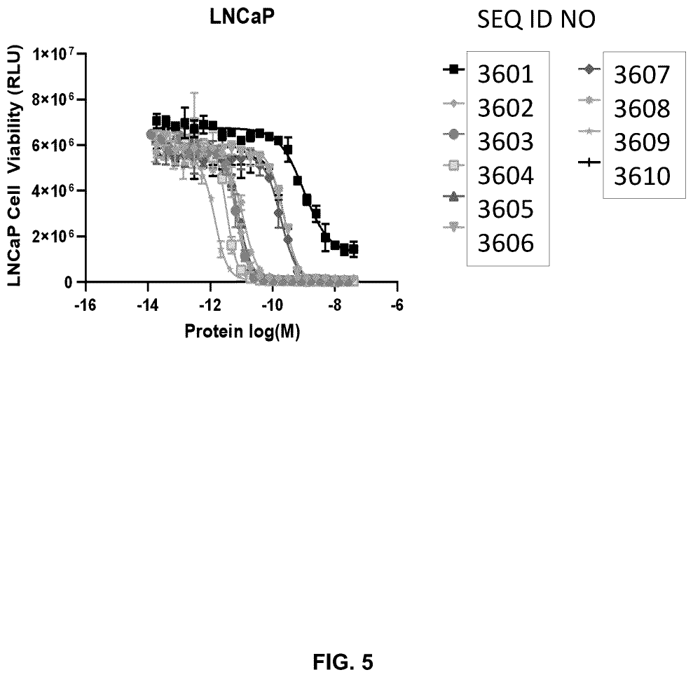

TDCC Assay: For functional potency in a T cell-dependent cellular cytotoxicity assays, soluble test molecules shown in Table 4 were incubated in multi-well plates, with purified resting human T cells (effector cell) and LNCaP cancer cells at 10:1 effector: target cell ratio for 48-72 h at 37° C. Human serum albumin is present. The cancer target cell lines had been stably transduced with a luciferase reporter gene to allow specific T cell-mediated cell killing measurement by Steady-Glo (Promega). Results shown in FIG. 5 demonstrates the masking effects of various peptide masks. The EC₅₀ values are provided in Table 4. The efficiency of masking is quantitated by the fold increase in EC₅₀ of SEQ ID Nos 3601 to 3608 and 3610 over SEQ ID NO 3609 (Table 4). The results show that masking sequences QDGNEE (SEQ ID NO: 3068), QDANEE (SEQ ID NO: 3672) and QEGNEE (SEQ ID NO: 3669) provide the most efficient masking effect.

### Example 5: MSLN Targeting Extended-Release Binding Proteins with Different Peptide Mask Sequences Induced T Cell-Dependent Cellular Cytotoxicity Assay

The binding proteins with various masking peptides used in this study are summarized in Table 5. The configuration of the anti-target, anti-albumin and anti-CD3 binding domains are according to FIG. 2A (SEQ ID NOS: 3659 to 3661) or FIG. 2D (SEQ ID NO: 3662).

TABLE 5
MSLN Binding Proteins and EC₅₀ values

| | | | | with | increase |
|---|---|---|---|---|---|
| | | | | BSA | in EC₅₀ |
| | | | | fold | with |
| | | | | increase | HSA |
| | | | | in EC₅₀ | fold |
| | | | | over | over |
| | | | | SEQ | SEQ |
| SEQ | | EC₅₀ | EC₅₀ | ID | ID |
| ID | | BSA | HSA | NO | NO |
| NO | Protein | (M) | (M) | 3662 | 3662 |
| 3659 | QEGNEE:NCLV: | 2E−11 | 9E−11 | 15 | 25 |
| | αMSLN:αALB: | | | | |
| | αCD3 | | | | |
| 3660 | QDANEE:NCLV: | 1E−11 | 1E−10 | 14 | 26 |
| | αMSLN:αALB: | | | | |
| | αCD3 | | | | |
| 3661 | QDGNEE:NCLV: | 1E−09 | 2E−09 | 1124 | 525 |
| | αMSLN:αALB: | | | | |
| | αCD3 | | | | |
| 3662 | stub:αMSLN: | 1E−12 | 4E−12 | 1 | 1 |
| | αALB:αCD3 | | | | |

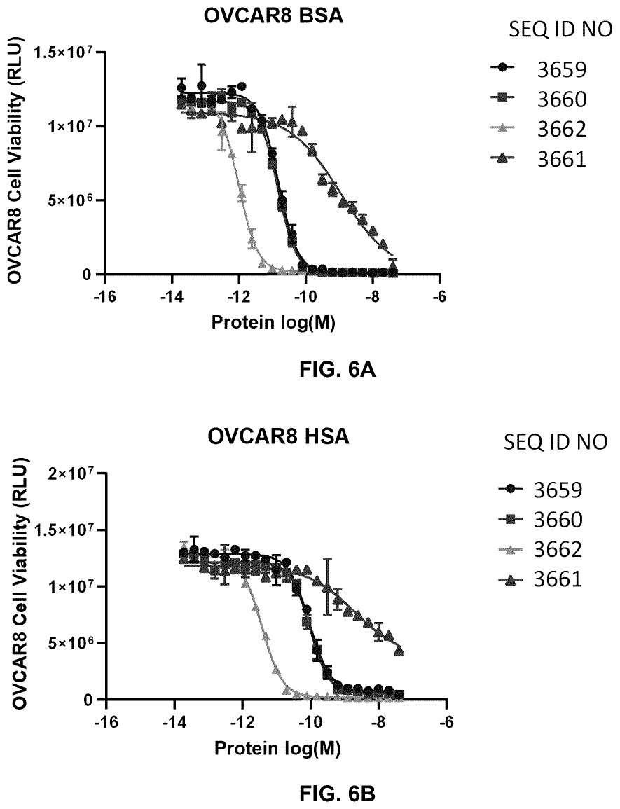

TCC Assay: or functional potency in a cell-dependent cellular cytotoxicity assays, soluble test molecules shown in Table 5 were incubated, in multi-well plates, with purified resting human T cells (effector cell) and OVCAR8 cancer cells at 10:1 effector: target cell ratio for 48 h at 37° C. Bovine serum albumin (BSA) or HSA were present at 15 mg/ml. The cancer target cell lines had been stably transduced with a luciferase reporter gene to allow specific T cell-mediated cell killing measurement by Steady-Glo (Promega). Results shown in FIGS. 6A-B demonstrates the masking effects of various peptide masks. The EC₅₀ values are provided in Table 5. The efficiency of masking is quantitated by the fold increase in EC₅₀ of SEQ ID NOS: 3659 to 3661 over SEQ ID NO 3663 (Table 5).

### Example 6: CD19 Targeting Extended-Release Binding Proteins with Different Domain Configurations Induced T Cell-Dependent Cellular Cytotoxicity Assay

The binding proteins used in this study are summarized in Table 6. The configuration of the anti-target, anti-albumin and anti-CD3 binding domains are according to FIGS. 2A-C and FIGS. 2G-I (SEQ ID NOS: 3627 to 3631) and FIGS. 2D-F and FIGS. 2J-L (SEQ ID NOS: 3632 to 3636). SEQ ID NOS: 3627 to 3631 contain a masking domain and anon-cleavable linker. SEQ ID NOS: 3632 to 3636 are genetically engineered to encode the product after protease cleavage if the molecules had been made with a cleavable linker (stub: Active molecules).

TABLE 6
CD19 Binding Proteins and EC₅₀ values

| NCLV | stub:Active | | NCLV | | |
|---|---|---|---|---|---|
| SEQ ID | SEQ ID | | EC50 | stub:Active | NCLV/ |
| NO | NO | Protein Name | (M) | EC50 (M) | stub:Active |
| 3626 | 3632 | αCD19 A:C:T | 1E−09 | 8E−11 | 17 |
| 3627 | 3633 | αCD19 A:T:C | 3E−10 | 3E−11 | 9 |
| 3628 | 3634 | αCD19 C:A:T | >1E−8 | 4E−11 | >270 |
| 3629 | 3635 | αCD19 C:T:A | >1E−8 | 1E−10 | >80 |
| 3630 | 3611 | αCD19 T:A:C | >1E−8 | 2E−11 | >420 |
| 3631 | 3636 | αCD19 T:C:A | >1E−8 | 1E−11 | >870 |

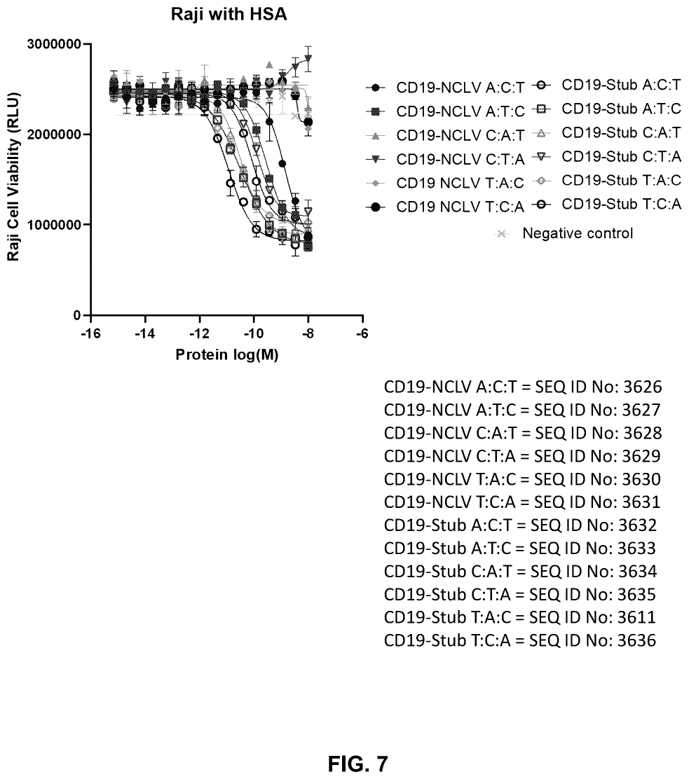

TDCC Assay: For functional potency in a T cell-dependent cellular cytotoxicity assays, soluble test molecules shown in Table 6 were incubated, in multi-well plates, with purified resting human T cells (effector cell) and Raji cancer cells at 10:1 effector: target cell ratio for 72 h at 37° C. HSA was present. The cancer target cell lines had been stably transduced with a luciferase reporter gene to allow specific T cell-mediated cell killing measurement by Steady-Glo (Promega). Results shown in FIG. 7 demonstrates the TDCC assay results of various CD19 targeting proteins. The EC₅₀ values are provided in Table 6. The efficiency of masking is quantitated by the fold increase in EC₅₀ of the NCLV protein over the stub:Active protein (Table 6). Results shows that T:A:C and T:C:A configurations have low double digit picomolar stub: active EC₅₀ and no activity when masked with a NCLV linker, while A:C:T and A:T:C configurations have poor masking effects.

### Example 7: FLT3 Targeting Extended-Release Binding Proteins with Different Domain Configurations Induced T Cell-Dependent Cellular Cytotoxicity Assay

The binding proteins used in this study are summarized in Table 7. The configuration of the anti-target, anti-albumin and anti-CD3 binding domains are according to FIGS. 2A-C and FIGS. 2G-I (SEQ ID NOS: 3648 to 3653) and FIGS. 2D-F and FIG. 2J-L (SEQ ID NOS: 3654 to 3658). SEQ ID NOS: 3648 to 3653 contain a masking domain and a non-cleavable linker. SEQ ID NOS: 3654 to 3658 are genetically engineered to encode the product after protease cleavage if the molecules had been made with a cleavable linker (stub:Active molecules). The FLT3 binder used in this study is 19C (SEQ ID NO: 1076).

TABLE 7
FLT3 Binding Proteins and EC₅₀ values

| NCLV | stub:Active | | NCLV | | NCLV/ |
|---|---|---|---|---|---|
| SEQ ID | SEQ ID | | EC50 | stub:Active | stub |
| NO | NO | Protein Name | (M) | EC50 (M) | Active |
| 3648 | 3654 | αFLT3-H19C A:C:T | >1E−8 | 2E−09 | >7 |
| 3649 | 3655 | αFLT3-H19C A:T:C | ~1E−8 | 8E−10 | ~13 |
| 3650 | 3656 | αFLT3-H19C C:A:T | >1E−8 | 2E−10 | >50 |
| 3651 | 3657 | αFLT3-H19C C:T:A | >1E−8 | 5E−10 | >20 |
| 3652 | 3613 | αFLT3-H19C T:A:C | >1E−8 | 5E−11 | >210 |
| 3653 | 3658 | αFLT3-H19C T:C:A | >1E−8 | 3E−10 | >30 |

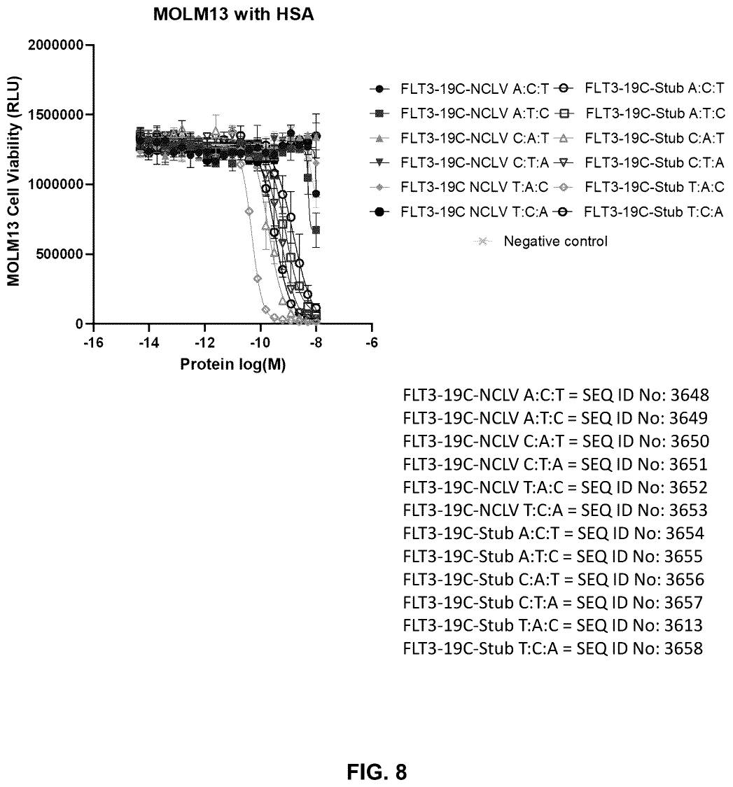

TDCC Assay: For functional potency in a T cell-dependent cellular cytotoxicity assays, soluble test molecules shown in Table 7 were incubated, in multi-well plates, with purified resting human T cells (effector cell) and MOLM13 cancer cells at 10:1 effector: target cell ratio for 48-72 h at 37° C. HSA was present. The cancer target cell lines had been stably transduced with a luciferase reporter gene to allow specific T cell-mediated cell killing measurement by Steady-Glo (Promega). Results shown in FIG. 8 demonstrates the TDCC assay results of various FLT3 targeting proteins. The EC₅₀ values are provided in Table 7. The efficiency of masking is quantitated by the fold increase in EC₅₀ of the NCLV protein over the stub:Active protein (Table 7). Results shows that TAC configuration has ˜50 picomolar EC₅₀ in the stub:Active form and no activity when masked with a NCLV linker, while A:C:T and A:T:C configurations have poor masking effects.

### Example 8: FLT3 Targeting Extended-Release Binding Proteins with Different Domain Configurations Induced T Cell-Dependent Cellular Cytotoxicity Assay

The binding proteins used in this study are summarized in Table 8. The configuration of the anti-target, anti-albumin and anti-CD3 binding domains are according to FIGS. 2A-C and FIGS. 2G-I (SEQ ID NOS 3637 to 3642) and FIGS. 2D-F and FIGS. 2J-L (SEQ ID NOS 3643 to 3647). SEQ ID NOS: 3637 to 3642 contain a masking domain and a non-cleavable linker. SEQ ID NOS 3643 to 3647 are genetically engineered to encode the product after protease cleavage if the molecules had been made with a cleavable linker (stub:Active molecules). The FLT3 binder used in this study is 107 (SEQ ID NO: 1074).

TABLE 8
FLT3 Binding Proteins and EC₅₀ values

| NCLV | stub:Active | | NCLV | | NCLV/ |
|---|---|---|---|---|---|
| SEQ ID | SEQ ID | | EC50 | stub:Active | stub |
| NO | NO | Protein Name | (M) | EC50 (M) | Active |
| 3637 | 3643; | αFLT3-H107 | 4E−10 | Not | N/A |
| | did not | A:C:T | | Tested | |
| | express | | | | |
| 3638 | 3644 | αFLT3-H107 | 3.6E−10 | 5E−12 | 70 |
| | | A:T:C | | | |
| 3639 | 3645 | αFLT3-H107 | >1E−8 | 7E−12 | >1500 |
| | | C:A:T | | | |
| 3640 | 3646 | αFLT3-H107 | >1E−8 | 5E−12 | >2000 |
| | | C:T:A | | | |
| 3641 | 3612 | αFLT3-H107 | >1E−8 | 8E−12 | >1200 |
| | | T:A:C | | | |
| 3642 | 3647 | αFLT3-H107 | >1E−8 | 7E−12 | >1500 |
| | | T:C:A | | | |

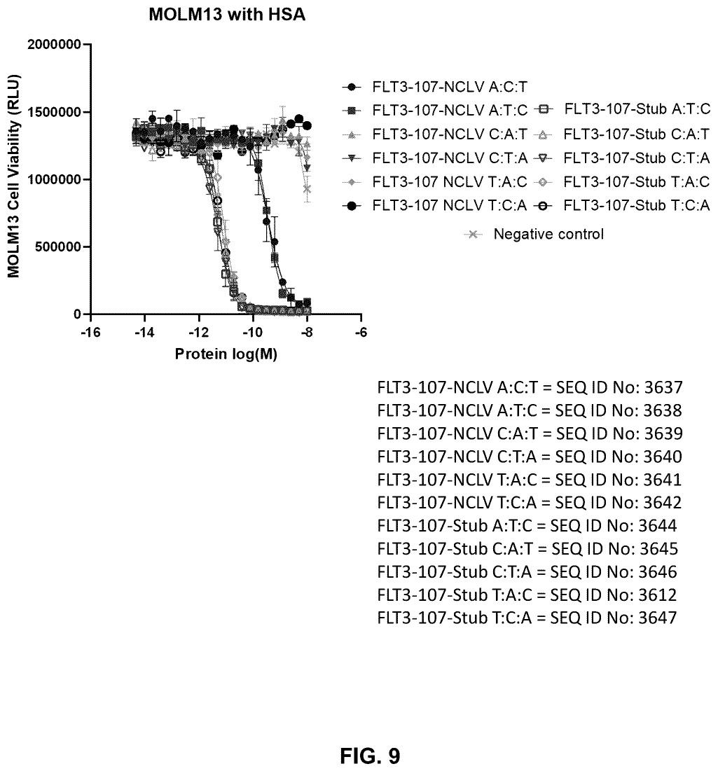

TDCC Assay: For functional potency in a T cell-dependent cellular cytotoxicity assays, soluble test molecules shown in Table 8 were incubated, in multi-well plates, with purified resting human T cells (effector cell) and MOLM13 cancer cells at 10:1 effector: target cell ratio for 48 h at 37° C. HSA was present. The cancer target cell lines had been stably transduced with a luciferase reporter gene to allow specific T cell-mediated cell killing measurement by Steady-Glo (Promega). Results shown in FIG. 9 demonstrates the TDCC assay results of various FLT3 targeting proteins. The EC₅₀ values are provided in Table 8. The efficiency of masking is quantitated by the fold increase in EC₅₀ of the NCLV protein over the stub:Active protein (Table 8). Results shows that 4 configurations (C:A:T, C:T:A, T:A:C and T:C:A) have single digit picomolar Active EC₅₀ in the active form and no activity when masked with a NCLV linker, while the A:T:C configuration has poor masking. The stub_A:C:T configuration did not express, so masking efficiency in the A:C:T configuration could not be assessed.

### Example 9: CD33 Targeting Extended-Release Binding Proteins with Different Domain Configurations Induced T Cell-Dependent Cellular Cytotoxicity Assay

The binding proteins used in this study are summarized in Table 9. The configuration of the anti-target, anti-albumin and anti-CD3 binding domains are according to FIGS. 2A-C and FIGS. 2G-I (SEQ ID NOS 3614 to 3619) and FIGS. 2D-F and FIG. 2J-L (SEQ ID NOS 3620 to 3625). SEQ ID NOS: 3614 to 3619 contain a masking domain and a non-cleavable linker. SEQ ID NOS 3620 to 3625 are genetically engineered to encode the product after protease cleavage if the molecules had been made with a cleavable linker (stub:Active molecules).

TABLE 9
CD33 Binding Proteins and EC₅₀ values

| NCLV | stub:Active | | NCLV | | |
|---|---|---|---|---|---|
| SEQ ID | SEQ ID | | EC50 | stub:Active | NCLV/ |
| NO | NO | Protein Name | (M) | EC50 (M) | stub:Active |
| 3614 | 3620 | αCD33 A:C:T | 2E−10 | 2E−11 | 13 |
| 3615 | 3621 | αCD33 A:T:C | 1E−10 | 4E−12 | 64 |
| 3616 | 3622 | αCD33 C:A:T | >1E−7 | 4E−11 | >2500 |
| 3617 | 3623 | αCD33 C:T:A | >1E−7 | 2E−11 | >4200 |
| 3618 | 3624 | αCD33 T:A:C | >1E−7 | 8E−11 | >1300 |
| 3619 | 3625 | αCD33 T:C:A | >1E−7 | 7E−12 | >15000 |

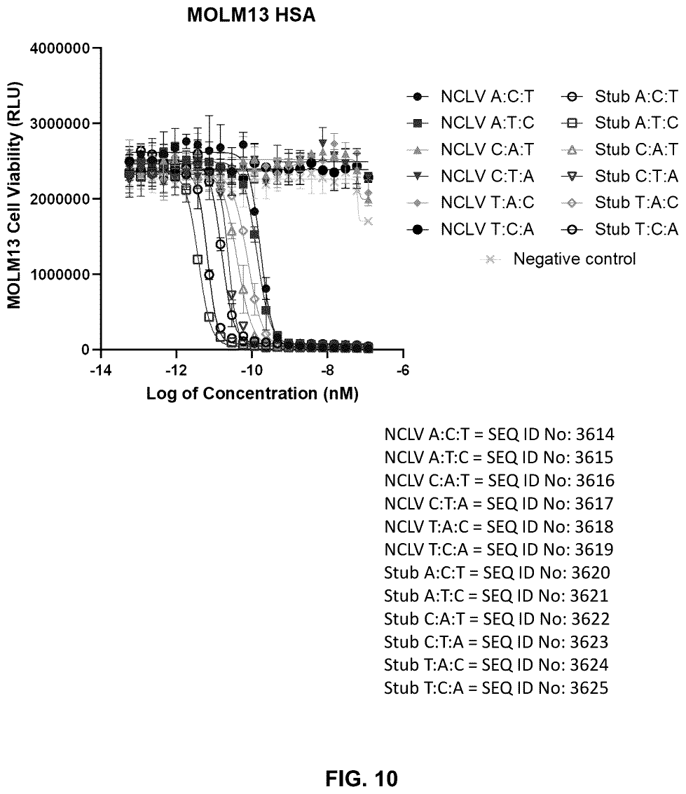

TDCC Assay: For functional potency in a T cell-dependent cellular cytotoxicity assays, soluble test molecules shown in Table 9 were incubated, in multi-well plates, with purified resting human T cells (effector cell) and MOLM13 cancer cells at 10:1 effector: target cell ratio for 48 h at 37° C. HSA was present. The cancer target cell lines had been stably transduced with a luciferase reporter gene to allow specific T cell-mediated cell killing measurement by Steady-Glo (Promega). Results shown in FIG. 10 demonstrates the TDCC assay results of various CD33 targeting proteins. The EC₅₀ values are provided in Table 9. The efficiency of masking is quantitated by the fold increase in EC₅₀ of the NCLV protein over the stub:Active protein (Table 9). Results shows that 4 configurations (C:A:T, C:T:A, T:A:C and T:C:A) have single digit picomolar Active EC₅₀ in the active form and no activity when masked with a NCLV linker, while A:C:T and A:T:C configurations have poor masking effects.

### Example 10: TDCC Activity of the Protease Activated Extended-Release Binding Proteins

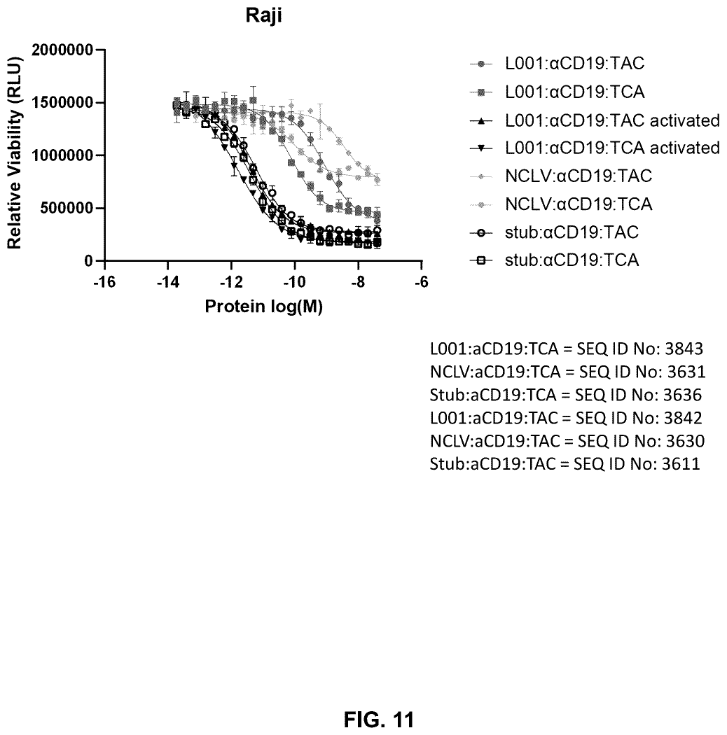

CD19-targeting extending-release binding proteins in the T:A:C or T:C:A (FIGS. 2A and B) configurations were expressed and purified. For each configuration, four different versions of protein were produced. The first versions (SEQ ID NOS: 3842 and 3843) contained a protease cleavage site, L001 (SEQ ID NO: 3688), between the masking domain (SEQ ID NO: 3663) and the CD19 targeting domain. The second versions were identical to the first version except that the protein was treated with the ST14 protease. The third versions (SEQ ID NOS: 3630 and 3631) contained a non-cleavage linker (SEQ ID NO: 3687) between the masking domain and the targeting domain. The fourth versions (SEQ ID NOS: 3611 and 3636) were engineered with the amino acid sequence VVGGGG (SEQ ID NO: 3879), referred to as “stub”, on the N-terminus of the target binding domain to product of an extended-binding release protein after protease cleavage (FIGS. 2D and E). These proteins were tested in a Raji TDCC assay as described in Example 6 and the Raji cell viability data versus the concentration of CD19 protein added is plotted in FIG. 11. As expected, the CD19 binding proteins activated with protease or the stub CD19 proteins potently and effectively directed the T cells to kill the Raji cells. CD19 binding proteins with a non-cleavage linker were unable to direct T cells to kill the Raji cells. CD19 proteins with a cleavable linker but not treated with protease were able to partially direct T cells to kill the Raji cells. It is hypothesized that during this 72 hour assay that these proteins became partially activated because of proteases produced by the T cells and/or Raji cells.

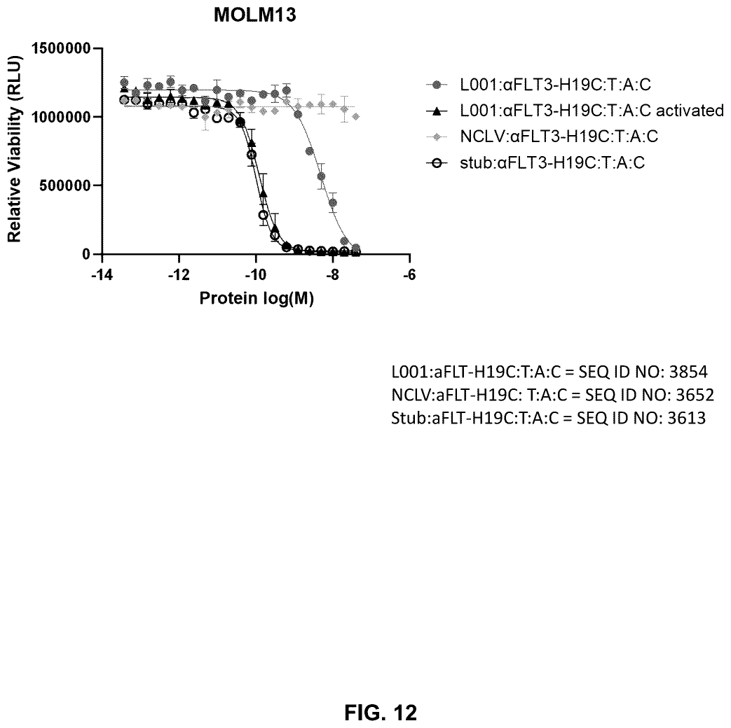

FLT3-targeting extending-release binding proteins in the T:A:C (FIG. 2A) configuration were expressed and purified. Four different versions of proteins were produced. The first version (SEQ ID NO: 3854) contained a protease cleavage linker L001 (SEQ ID NO: 3688) (and flanking sequence at the N-terminal and C-terminal of L001), between the masking domain (SEQ ID NO: 3633) and the FLT3 targeting domain. The second version was identical to the first version except that the protein was treated with the ST14 protease. The third version (SEQ ID NO: 3652) contained a non-cleavage linker (SEQ ID NO: 3687) between the masking domain and the targeting domain. The fourth version (SEQ ID NO: 3613) was engineered with the amino acid sequence VVGGGG (SEQ ID NO: 3879), referred to as “stub”, on the N-terminus of the target binding domain to product of an extended-binding release protein after protease cleavage (FIG. 2D). These proteins were tested in a MOLM13 TDCC assay as described in Example 6 and the MOLM13 cell viability data versus the concentration of FLT3 protein added is plotted in FIG. 12. As expected, the FLT3 binding protein activated with protease or the stub FLT3 protein potently and effectively directed the T cells to kill the MOLM13 cells. FLT3 binding protein with a non-cleavage linker was unable to direct T cells to kill the MOLM13 cells. FLT3 protein with a cleavable linker but not treated with protease were able to direct T cells to kill the MOLM13 cells but with less potency than the stub or protease activated proteins. It is hypothesized that during this 72 hour assay that these proteins became partially activated because of proteases produced by the T cells and/or MOLM13 cells.

### Example 11: Binding of TriTAC-XR Prodrug and Activated Drug to Human CD3ε by ELISA

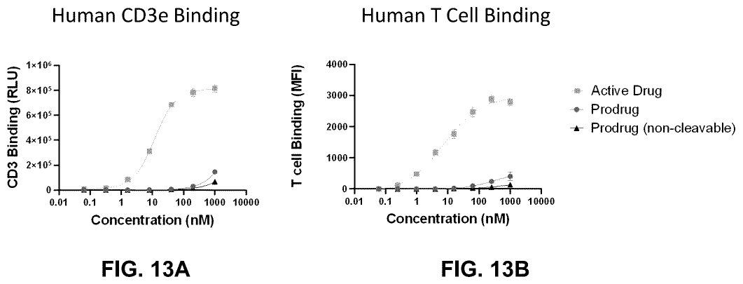

Exemplary TriTAC-XR with a non-cleavable (NCLV) linker (SEQ ID NO: 3630), a cleavable linker (SEQ ID NO: 3876), or an stub:Active control (SEQ ID NO: 3611) were assessed for binding to recombinant human CD3ε using ELISA. The results are shown in FIG. 13A. The mask reduced the binding to human CD3ε by more than 100-fold.

### Example 12: Binding of TriTAC-XR Prodrug and Activated Drug to Human T Cells by Flow Cytometry

Exemplary TriTAC-XR with a non-cleavable (NCLV) linker (SEQ ID NO: 3630), a cleavable linker (SEQ ID NO: 3876), or an stub:Active control (SEQ ID NO: 3611) were assessed for binding to human T cells using flow cytometry. The results are shown in FIG. 13B. The mask reduced the binding to human T cells by more than 100-fold.

### Example 13: Pharmacokinetics and Pharmacodynamics of FLT3 TriTAC-XR in Cynomolgus Monkeys

FLT3 TriTAC-XR-L001 (SEQ ID NO: 3873) or FLT3 TriTAC-XR-L085 (SEQ ID NO: 3874) was administered via single i.v. bolus injection to cynomolgus monkeys. FLT3 TriTAC-XR-L001 was administered at 300 and 1000 μg/kg and FLT3 TriTAC-XR-L085 was administered at 1000 μg/kg with two test subjects per dose group. FLT3 TriTAC binds to cynomolgus FLT3, CD3, and albumin with affinities of 2.7 nM, 3.3 nM and 4.4 nM, respectively, as determined using biolayer interferometry. FLT3 TriTAC-XR molecules have similar affinity to FLT3 and albumin as TriTAC, but reduced binding to CD3 due to the presence of a peptide mask, see examples 11 and 12.

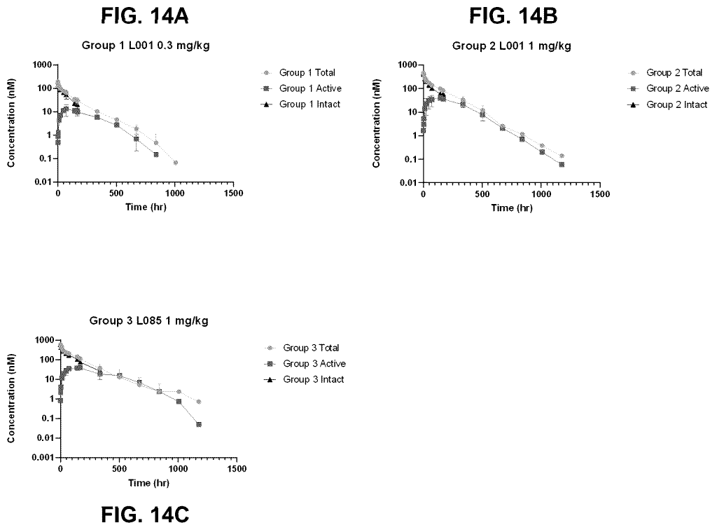

The total amount of intact and active FLT3 TriTAC-XR (L001, SEQ ID NO: 3873, and L085, SEQ ID NO: 3874) present in serum samples was measured using an electrochemiluminescent ELISA assay using labeled antibodies recognizing non-overlapping epitopes on the anti-ALB domain of the TriTAC-XR molecule as capture and detection reagents. The amount of activated FLT3 TriTAC-XR present in serum samples was measured using an electrochemiluminescent ELISA assay using labeled antibodies recognizing the anti-ALB domain and the anti-CD3 domain of the TriTAC-XR molecule as capture and detection reagents. The antibody recognizing the anti-CD3 domain is blocked by the mask in the intact prodrug, resulting in 400× preferential detection of active drug. The amount of intact prodrug was calculated by subtracting the active drug concentration from the total drug concentration. The measured and calculated serum concentrations versus time are plotted in FIGS. 14A-14C. The active form of the drug formed slowly after dosing, reaching a maximum concentration approximately 72 to 144 hours after dosing.

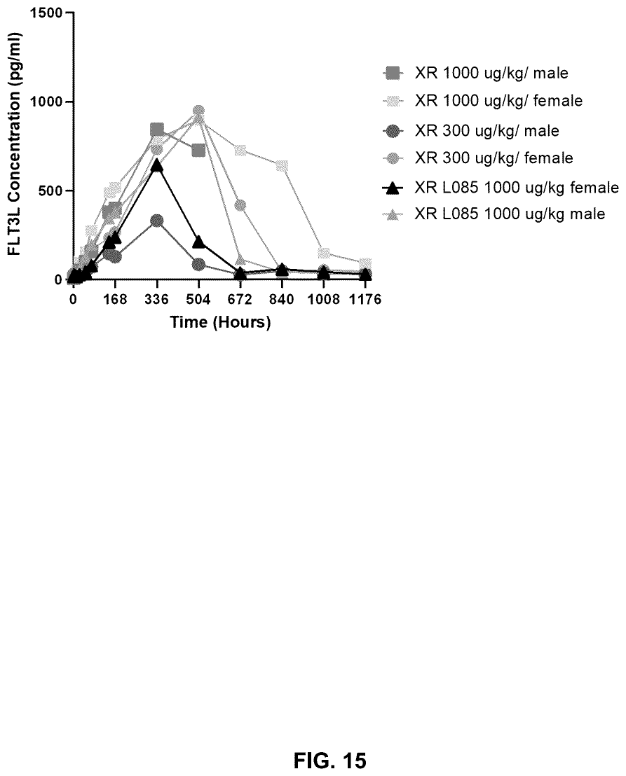

To determine if the FLT3 TriTAC-XR directed cynomolgus T cells to kill endogenous cynomolgus FLT3-expressing cells, soluble FLT3L in serum and FLT3 RNA in bone marrow were measured in samples collected from the pharmacokinetic study described above. Depletion of FLT3-expressing cells should result in an increase in soluble FLT3L (Brauchle et al., Mol Cancer Ther 2020; 19:1875-88). An electrochemiluminescent ELISA specific for Non-Human Primate FLT3L (Meso Scale Discovery) was used to measure the levels of FLT3L in serum samples collected at different time points (FIG. 15). For all dose groups, the soluble FLT3L increased for the first 336 to 504 hours before declining back to baseline by 672 to 1176 hours.

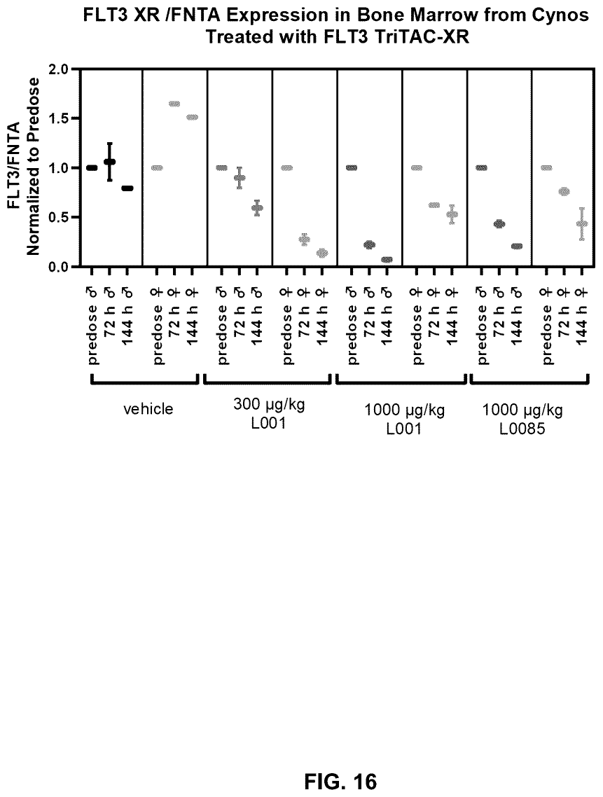

If FLT3 expressing cells are depleted from bone marrow, then FLT3 transcripts should be depleted from RNA purified from bone marrow. RNA was purified from bone marrow using kits (Qiagen), cDNA was prepared by a reverse transcriptase reaction, and qPCR was used to measure the amount of FLT3 present using a standard curve qPCR method. FNTA was used as a house keeping gene for the qPCR reactions. Plotted in FIG. 16 are FTL3 RNA levels normalized to FNTA for bone marrow samples isolated from animals treated with TriTAC-XR. Compared to samples collected prior to dosing, FLT3 RNA is greatly reduced at all time points measured. The combined FLT3L (FIG. 15) and FLT3 RNA (FIG. 16) data indicate that the FLT3 TriTAC-XR-L001 (SEQ ID NO: 3873) and FLT3 TriTAC-XR-L085 (SEQ ID NO: 3874) reduced FLT3 expressing cells in cynomolgus monkeys.

### Example 14: Pharmacokinetics of FLT3 TriTAC in Cynomolgus Monkeys

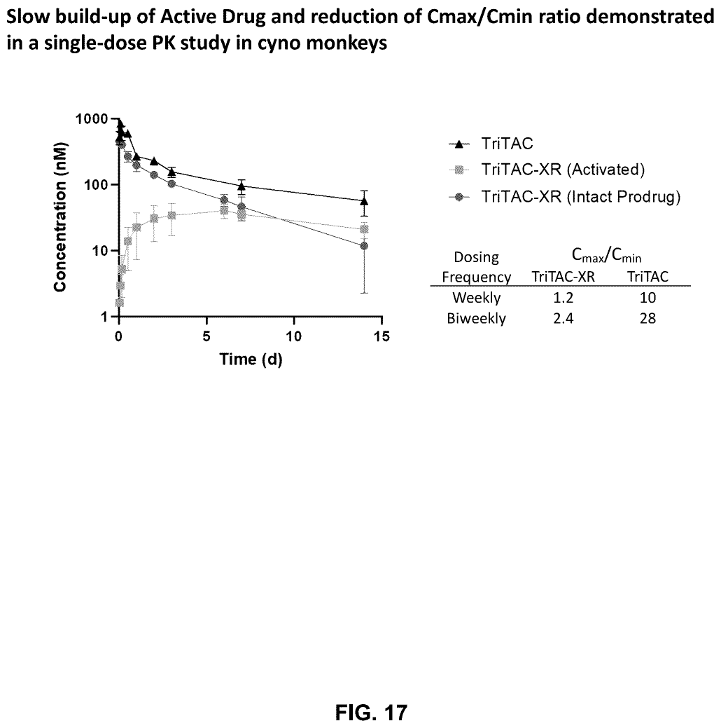

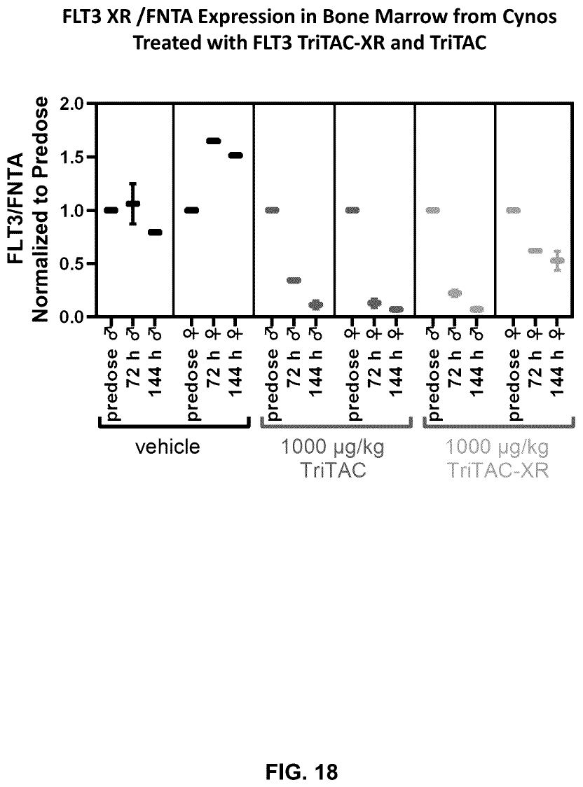

FLT3 TriTAC (SEQ ID NO: 3875) was administered via single i.v. bolus injection to cynomolgus monkeys at 1 mg/kg. Serum concentrations of the TriTAC were measured for 15 days at the indicated timepoints (FIG. 17). To compare the FLT3 TriTAC with TriTAC-XR-L001, the first 15 days of FLT3 TriTAC-XR data from Example 13 were plotted in FIG. 18. The results show that TriTAC-XR resulted in a slow build-up of active drug, and a reduced Cmax/Cmin ratio for the active drug (FIG. 17) compared to the TriTAC. Plotted in FIG. 18 are FTL3 RNA levels normalized to FNTA for bone marrow samples isolated from animals treated with TriTAC or TriTAC-XR (from Example 13). The FLT3 transcription levels in animals dosed i.v. with 1000 μg/kg FLT3 TriTAC-XR-L001 (SEQ ID NO: 3873) were compared to the FLT3 transcription levels in animals dosed i.v. with a constitutively active FLT3 TriTAC (SEQ ID NO: 3875). Compared to samples collected prior to dosing, FLT3 RNA is greatly reduced at all time points measured. The FLT3 RNA (FIG. 18) data indicate that the FLT3 TriTAC-XR-L001 (SEQ ID NO: 3873) and the constitutively active FLT3 TriTAC (SEQ ID NO: 3875) reduced FLT3 expressing cells in cynomolgus monkeys.

### Example 15: Safety and Efficacy of FLT3 TriTAC-XR in Cynomolgus Monkeys

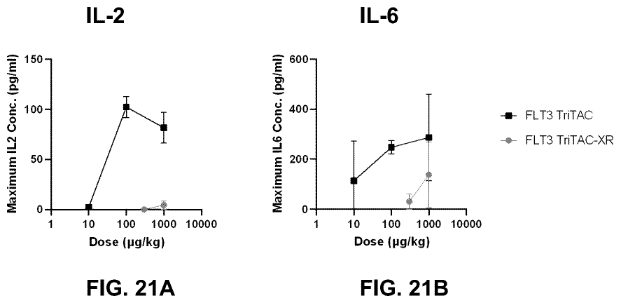

To assess the safety improvement of TriTAC-XR, cytokines were measured at multiple timepoints between 2 and 48 hours after dosing. Cytokines levels were quantified using electrochemiluminescent ELISAs specific for Non-Human Primate IL-2 and IL-6 (Meso Scale Discovery). The peak cytokine levels in animals dosed i.v. with 300 and 1000 μg/kg FLT3 TriTAC-XR (FLT3 TriTAC-XR-L001, SEQ ID NO: 3873) were compared to the peak cytokine levels in animals dosed i.v. with a constitutively active FLT3 TriTAC (SEQ ID NO 3875) at 10, 100, and 1000 μg/kg and are shown in FIGS. 21A and 21B (IL-2 in FIG. 21A, IL-6 in FIG. 21B). Significant cytokines were generated from the constitutively active TriTAC at 100-fold lower doses than the TriTAC-XR-L001.

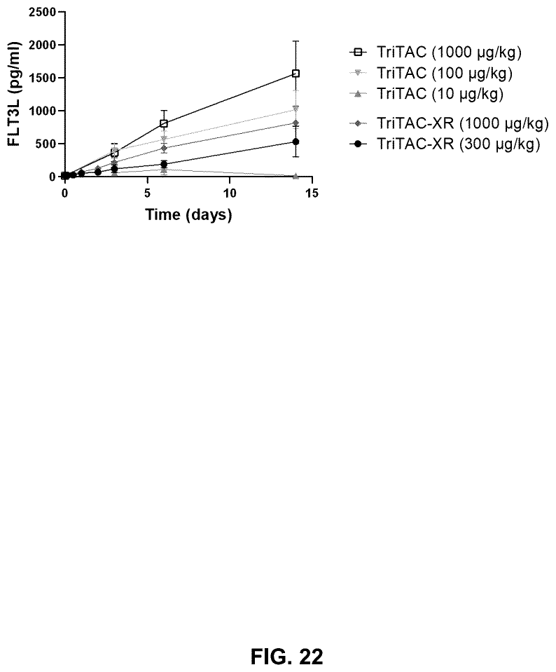

To assess the efficacy of TriTAC-XR, soluble FLT3L in serum were measured in samples collected from the safety study described above. Depletion of FLT3-expressing cells should result in an increase in soluble FLT3L (Brauchle et al. Mol Cancer Ther 2020; 19:1875-88). An electrochemiluminescent ELISA specific for Non-Human Primate FLT3L (Meso Scale Discovery) was used to measure the levels of FLT3L in serum samples collected at the indicated timepoints after dosing (FIG. 22). The soluble FLT3L levels in animals dosed i.v. with 300 and 1000 μg/kg FLT3 TriTAC-XR (FLT3 TriTAC-XR-L001, SEQ ID NO: 3873) were compared to the soluble FLT3L levels in animals dosed i.v. with a constitutively active FLT3 TriTAC (SEQ ID NO: 3875) at 10, 100, and 1000 μg/kg and are shown in FIG. 22. FLT3L was significantly elevated after administration of 100 and 1000 μg/kg TriTAC and 300 and 1000 μg/kg TriTAC-XR. (FIG. 22).

### Example 16: Efficacy of FLT3 TriTAC-XR in Mouse

All animal experiments were conducted according to the protocol approved by Institutional Animal Care and Use Committee of Harpoon Therapeutics (protocol number HAR-001-2019). Animals were purchased from The Jackson Laboratory then housed in a pathogen free animal facility located at Harpoon Therapeutics in accordance with IACUC guidelines. All studies were performed in NSG™ (NOD-scid IL2Rgamma^{null}) female mice 8 weeks of age with n=5 mice per group.

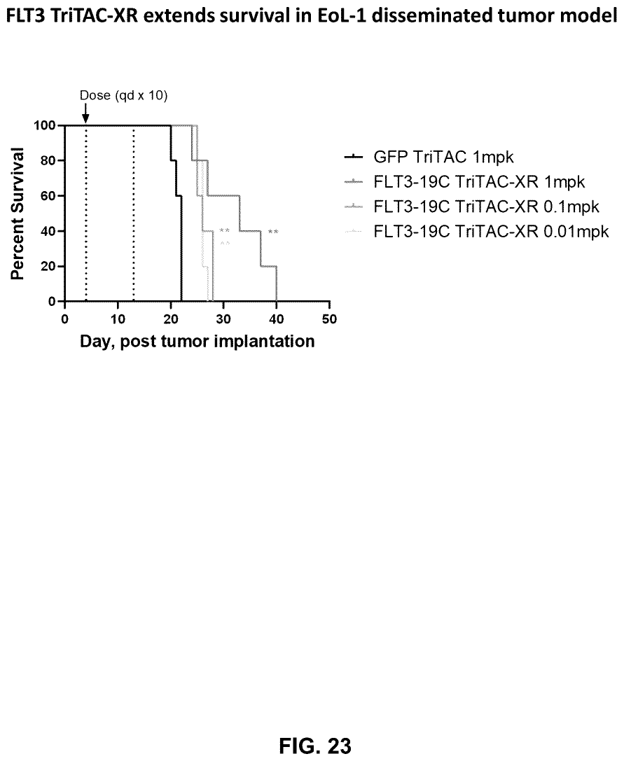

EoL-1 human eosinophilic leukemia cells (2×10⁶) were implanted intravenously in NSG™ mice on Day 0, followed by intraperitoneally implanted activated and expanded human T cells (2×10⁷) on Day 2. On Day 4, mice were administered a repeat intraperitoneal dose (q.d.×10) of non-targeting GFP TriTAC (SEQ ID NO: 3877) or FLT3 targeting TriTAC-XR (SEQ ID NO: 3873). Clinical observations were recorded and body weight was monitored at least three times weekly. Survival analysis of EoL-1 disseminated mouse model demonstrated FLT3 TriTAC-XR extends survival (FIG. 23). Statistical significance was determined using Survival Curve Comparisons Log-rank (Mantel-Cox) and Gehan-Breslow-Wilcoxon tests, all groups compared to control non-targeting GFP TriTAC (**, P<0.01, FIG. 23).

### Example 17: Pharmacokinetics of CD19 TriTAC-XR in Cynomolgus Monkeys

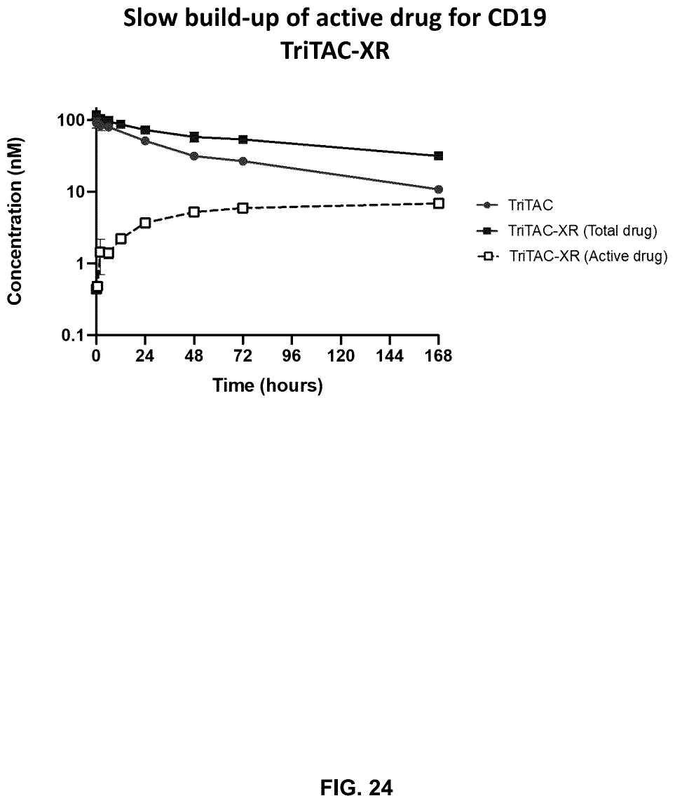

CD19 TriTAC-XR (CD19 TriTAC-XR-L001, SEQ ID NO: 3842) or a constitutively active CD19 TriTAC (SEQ ID NO: 3611) was administered via single i.v. bolus injection to cynomolgus monkeys at 0.3 mg/kg. Serum concentrations of each drug were measured for 168 hours at the indicated timepoints (FIG. 24). For TriTAC-XR, two assays were used to quantify the Intact Prodrug and the Activated drug. After administration, the Intact Prodrug of TriTAC-XR is slowly cleaved, generating Active drug. The results show that TriTAC-XR resulted in a slow build-up of active drug, (FIG. 24) compared to the TriTAC.

### Example 18: Safety and Efficacy of CD19 TriTAC-XR in Cynomolgus Monkeys

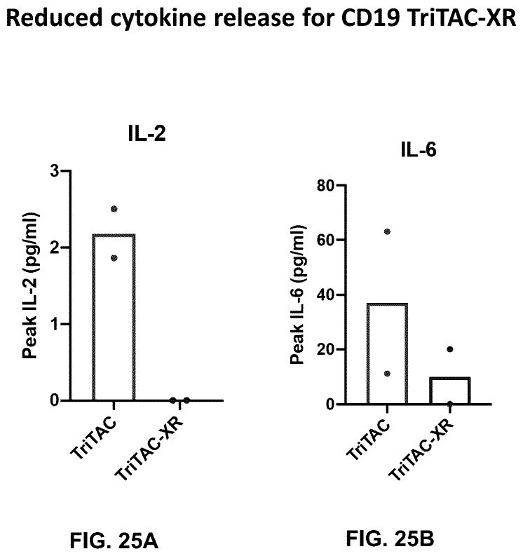

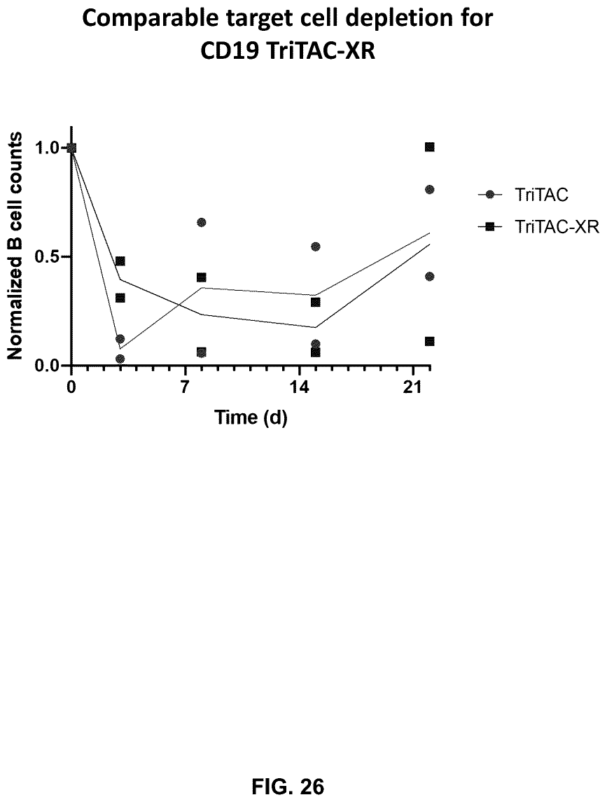

To assess the safety improvement of TriTAC-XR, cytokines were measured at multiple timepoints after dosing. Cytokines levels were quantified using electrochemiluminescent ELISAs specific for Non-Human Primate IL2 and IL6 (Meso Scale Discovery). The peak cytokine levels in animals dosed i.v. with 300 μg/kg CD19 TriTAC-XR (CD19 TriTAC-XR-L001, SEQ ID NO: 3842) were compared to the peak cytokine levels in animals dosed i.v. with 300 μg/kg of a constitutively active CD19 TriTAC (SEQ ID NO: 3611) are shown in FIGS. 25A and B (IL-2 in FIG. 25A, IL-6 in FIG. 25B). Significant cytokines were generated from the constitutively active TriTAC. FIG. 26 shows that target cell (B cell) depletion is comparable between CD19 TriTAC and CD19 TriTAC-XR.

### Example 19: Pharmacokinetics of CD20 TriTAC-XR in Cynomolgus Monkeys

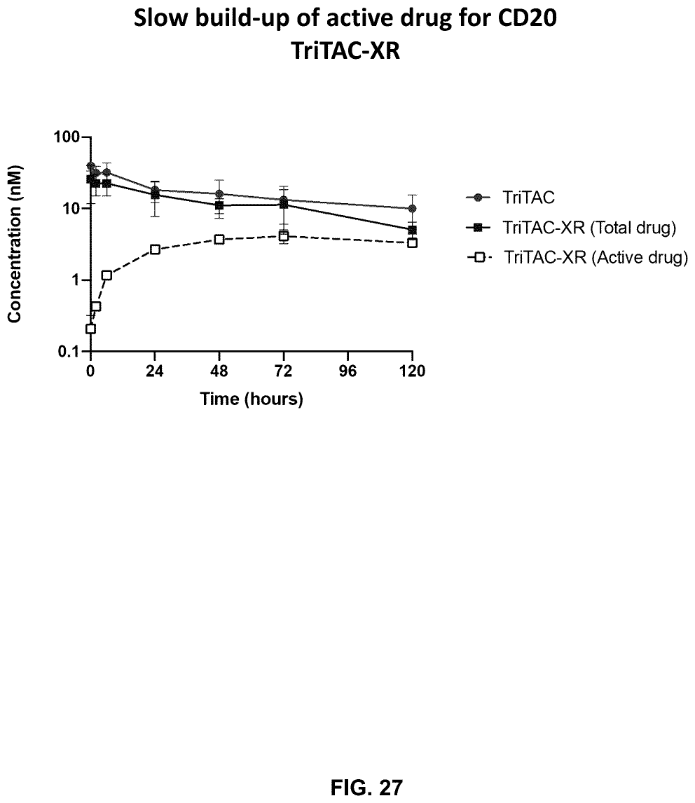

CD20 TriTAC-XR (CD20 TriTAC-XR-L001, SEQ ID NO: 3882) or a constitutively active CD20 TriTAC (SEQ ID NO: 3881) was administered via single i.v. bolus injection to cynomolgus monkeys at 0.03 mg/kg. Serum concentrations of each drug were measured for 120 hours at the indicated timepoints (FIG. 27). For TriTAC-XR, two assays were used to quantify the Intact Prodrug and the Activated drug. After administration, the Intact Prodrug of TriTAC-XR is slowly cleaved, generating Active drug. The results show that TriTAC-XR resulted in a slow build-up of active drug, (FIG. 27) compared to the TriTAC.

### Example 20: Safety and Efficacy of CD20 TriTAC-XR in Cynomolgus Monkeys

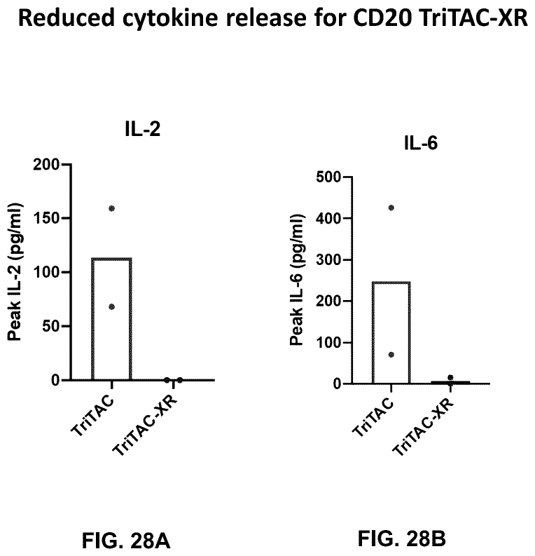

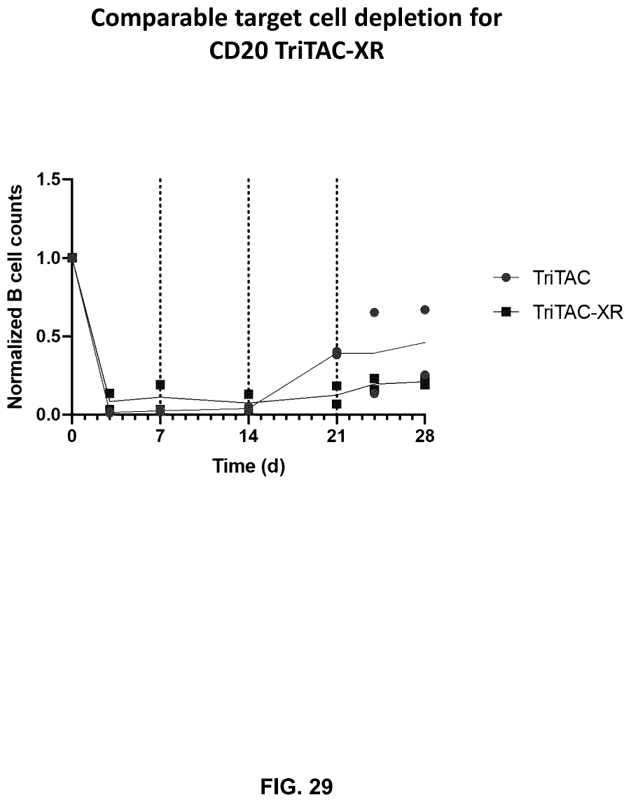

To assess the safety improvement of TriTAC-XR, cytokines were measured at multiple timepoints after dosing. Cytokines levels were quantified using electrochemiluminescent ELISAs specific for Non-Human Primate IL2 and IL6 (Meso Scale Discovery). The peak cytokine levels in animals dosed i.v. with 30 μg/kg CD20 TriTAC-XR (CD20 TriTAC-XR-L001, SEQ ID NO: 3882) were compared to the peak cytokine levels in animals dosed i.v. with a constitutively active CD20 TriTAC (SEQ ID NO: 3881) are shown in FIGS. 28A and B (IL-2 in FIG. 28A, IL-6 in FIG. 28B). Significant cytokines were generated from the constitutively active TriTAC. FIG. 29 shows that target cell (B cell) depletion is comparable between CD20 TriTAC and CD20 TriTAC-XR.

### Example 21. Predicted and Empirical PK Profiles

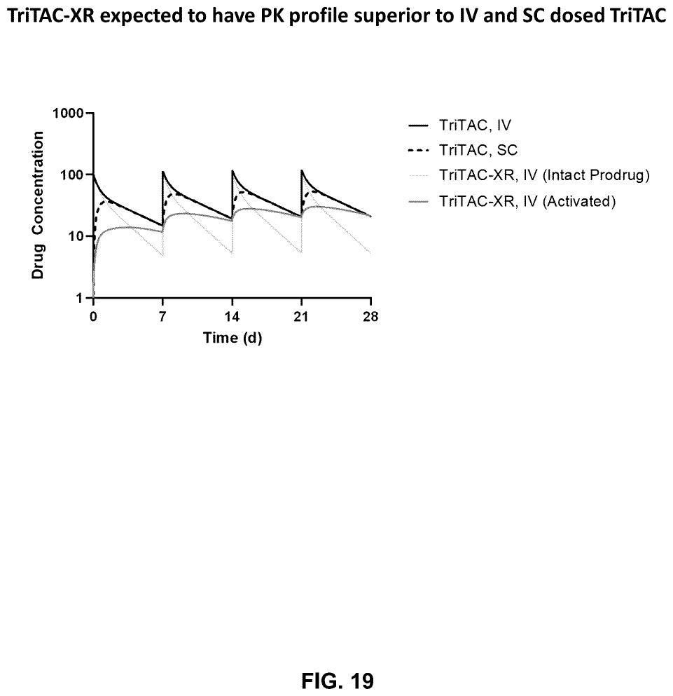

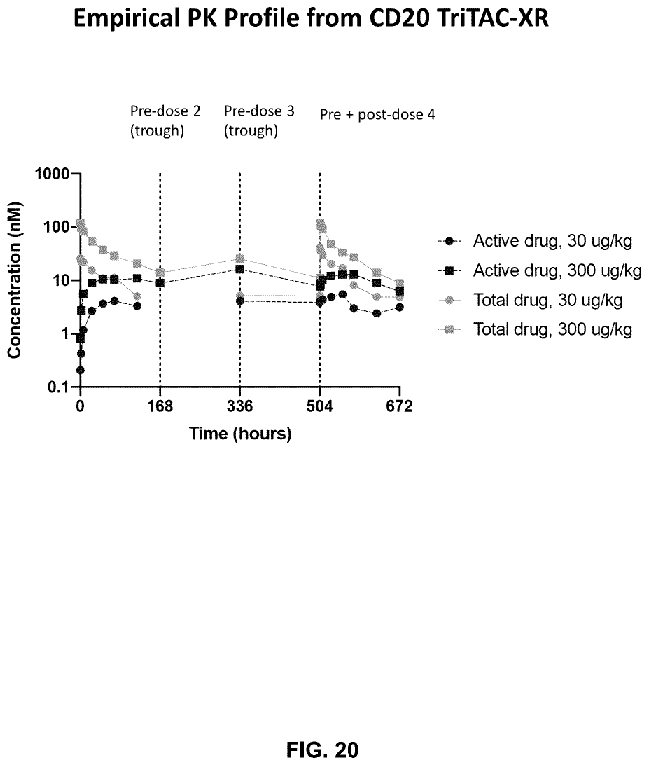

Based on the observation above, weekly repeat dosing of (1) intravenous administered TriTAC, (2) subcutaneous administered TriTAC, (3) and intravenous administered TriTAC-XR was modeled from single dose cynomolgus monkey pharmacokinetics data (FIG. 19). In vivo activated TriTAC-XR is predicted to have a lower Cmax, but similar Cmin to intravenous administered or subcutaneous administered TriTAC (FIG. 19). Repeated dosing of CD20 TriTAC-XR in cyno monkeys show a PK profile similar to the predicted PK profile where concentration of TriTAC-XR active drug in circulation (“apparent half-life”) is near constant due to continual conversion of prodrug to active drug (FIG. 20).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Sequence Listing Tables

Lengthy table referenced here
US20240216518A1-20240704-T00001
Please refer to the end of the specification for access instructions.

Lengthy table referenced here
US20240216518A1-20240704-T00002
Please refer to the end of the specification for access instructions.

Lengthy table referenced here
US20240216518A1-20240704-T00003
Please refer to the end of the specification for access instructions.

Lengthy table referenced here
US20240216518A1-20240704-T00004
Please refer to the end of the specification for access instructions.

Lengthy table referenced here
US20240216518A1-20240704-T00005
Please refer to the end of the specification for access instructions.

Lengthy table referenced here
US20240216518A1-20240704-T00006
Please refer to the end of the specification for access instructions.

Lengthy table referenced here
US20240216518A1-20240704-T00007
Please refer to the end of the specification for access instructions.

Lengthy table referenced here
US20240216518A1-20240704-T00008
Please refer to the end of the specification for access instructions.

Lengthy table referenced here
US20240216518A1-20240704-T00009
Please refer to the end of the specification for access instructions.

Lengthy table referenced here
US20240216518A1-20240704-T00010
Please refer to the end of the specification for access instructions.

Lengthy table referenced here
US20240216518A1-20240704-T00011
Please refer to the end of the specification for access instructions.

Lengthy table referenced here
US20240216518A1-20240704-T00012
Please refer to the end of the specification for access instructions.

Lengthy table referenced here
US20240216518A1-20240704-T00013
Please refer to the end of the specification for access instructions.

Lengthy table referenced here
US20240216518A1-20240704-T00014
Please refer to the end of the specification for access instructions.

Lengthy table referenced here
US20240216518A1-20240704-T00015
Please refer to the end of the specification for access instructions.

Lengthy table referenced here
US20240216518A1-20240704-T00016
Please refer to the end of the specification for access instructions.

Lengthy table referenced here
US20240216518A1-20240704-T00017
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240216518A1). An electronic copy of the table will also be available from the USPTO upon request and payment of the fee set forth in 37 CFR 1.19(b)(3).

1-154. (canceled)

155. A pharmaceutical composition comprising an extended-release binding protein, wherein the extended-release binding protein comprises a half-life extended immune cell engaging protein, a masking peptide, and a cleavable linker; wherein the half-life extended immune cell engaging protein comprises a first domain (A), a second domain (B), and a third domain (C), wherein:
(i) the first domain (A) comprises an immune cell engaging domain,
(ii) the second domain (B) comprises a half-life extended domain, and
(iii) the third domain (C) specifically binds to a target antigen;
wherein the cleavable linker covalently links the masking peptide to the N-terminus or the C-terminus of the half-life-extended immune cell engaging protein, and
wherein the cleavable linker is significantly cleaved in systemic circulation.

156. The pharmaceutical composition of claim 155, wherein the domains are linked in one of the following orders: H₂N-(C)-(B)-(A)-COOH, H₂N-(A)-(B)-(C)-COOH, H₂N-(B)-(A)-(C)-COOH, H₂N-(C)-(A)-(B)-COOH, H₂N-(A)-(C)-(B)-COOH, H₂N-(B)-(C)-(A)-COOH, or by linkers L1 and L2 in one of the following orders: H₂N-(C)-L1-(B)-L2(A)-COOH, H₂N-(A)-L1-(B)-L2-(C)-COOH, H₂N-(B)-L1-(A)-L2-(C)-COOH, H₂N-(C)-L1-(A)-L2-(B)-COOH, H₂N-(A)-L1-(C)-L2(B)-COOH, H₂N-(B)-L1-(C)-L2-(A)-COOH.

157. The pharmaceutical composition of claim 156, wherein the domains of the half-life extended immune cell engaging protein are linked in one of the following orders: H₂N-(C)-(B)-(A)-COOH, H₂N-(C)-(A)-(B)-COOH, or by linkers L1 and L2 in one of the following orders: H₂N-(C)-L1-(B)-L2-(A)-COOH, H₂N-(C)-L1-(A)-L2-(B)-COOH.

158. The pharmaceutical composition of claim 155, wherein the immune cell engaging domain comprises a natural killer (NK) cell engaging domain, a T cell engaging domain, a NK-T cell engaging domain, a B cell engaging domain, a dendritic cell engaging domain, a macrophage cell engaging domain, or a combination thereof.

159. The pharmaceutical composition of claim 158, wherein the immune cell engaging domain comprises the T cell engaging domain.

160. The pharmaceutical composition of claim 159, wherein the T cell engaging domain binds a CD3 molecule, and wherein the CD3 molecule is at least one of: a CD3γ molecule, a CD3δ molecule, or a CD3ε molecule.

161. The pharmaceutical composition of claim 155, wherein the first domain comprises a single-chain variable fragment (scFv) specific to human CD3;
wherein the scFv comprises a variable heavy chain region (VH), a variable light chain region (VL), and a linker, wherein VH comprises complementarity determining regions HC CDR1, HC CDR2, and HC CDR3, and wherein VL comprises complementarity determining regions LC CDR1, LC CDR2, and LC CDR3; and
wherein HC CDR1 comprises an amino acid sequence selected from the group consisting of SEQ ID NOs: 3081 and 3087-3098, the HC CDR2 comprises an amino acid sequence selected from the group consisting of SEQ ID NOs: 3082 and 3099-3109, the HC CDR3 comprises an amino acid sequence selected from the group consisting of SEQ ID NOs: 3083 and 3110-3119; and
wherein the LC CDR1 comprises an amino acid sequence selected from the group consisting of SEQ ID NOs: 3084 and 3120-3132, the LC CDR2 comprises an amino acid sequence selected from the group consisting of SEQ ID NOs: 3085 and 3099-3109, the LC CDR3 comprises an amino acid sequence selected from the group consisting of SEQ ID NOs: 3086 and 3146-3152.

162. The pharmaceutical composition of claim 161, wherein the HC CDR1 comprises the amino acid sequence of SEQ ID NO: 3097, the HC CDR2 comprises the amino acid sequence of SEQ ID NO: 3108, the HC CDR3 comprises the amino acid sequence of SEQ ID NO: 3110; and wherein the LC CDR1 comprises the amino acid sequence of SEQ ID NO: 3120, the LC CDR2 comprises the amino acid sequence of SEQ ID NO: 3145, the LC CDR3 comprises the amino acid sequence of SEQ ID NO: 3146.

163. The pharmaceutical composition of claim 155, wherein the first domain comprises an amino acid sequence selected from the group consisting of SEQ ID NOS: 3153-3169.

164. The pharmaceutical composition of claim 155, wherein the second domain comprises a single domain antibody (sdAb) which specifically binds to HSA;
wherein the sdAb comprises complementarity determining regions CDR1, CDR2, and CDR3, wherein the CDR1 comprises an amino acid sequence selected from the group consisting of SEQ ID NOS: 3170 and 3173-3175, the CDR2 comprises an amino acid sequence selected from the group consisting of SEQ ID NOS: 3171 and 3176-3181, the CDR3 comprises an amino acid sequence selected from the group consisting of SEQ ID NOS: 3172 and 8182-3183.

165. The pharmaceutical composition of claim 155, wherein the second domain comprises an amino acid sequence selected from the group consisting of SEQ ID NOs: 3184-3193.

166. The pharmaceutical composition of claim 155, wherein the masking peptide inhibits or reduces the binding of the first domain (A) to the human CD3 or the binding of the third domain (C) to the target antigen.

167. The pharmaceutical composition of claim 166, wherein the masking peptide comprises an amino acid sequence having an amino acid sequence selected from the group consisting of SEQ ID NOS: 3663-3682.

168. The pharmaceutical composition of claim 155, wherein the cleavable linker comprises a cleavage site recognizable by a protease, and wherein the protease is selected from the group consisting of a serine protease, a cysteine protease, an aspartate protease, a threonine protease, a glutamic acid protease, a metalloproteinase, a gelatinase, and an asparagine peptide lyase.

169. The pharmaceutical composition of claim 155, wherein the cleavable linker comprises an amino acid sequence having at least 80% homology to any one of SEQ ID NOs: 3688-3770 and 3878.

170. The pharmaceutical composition of claim 155, wherein the target antigen is a tumor antigen.

171. The pharmaceutical composition of claim 170, wherein the target antigen comprises CD19, CD20, CD33, FLT3, PSMA, MSLN, BCMA, DLL3, EGFR, or EpCAM.

172. The pharmaceutical composition of claim 171, wherein the third domain comprises an amino acid sequence having at least 80% homology to any one of SEQ ID NOs: 346-461, 476-489, 607-650, 798-846, 961-1079, 1308-1750, 3495-3496, 3771-3792, 3793-3808 3809-3823, and 3880.

173. The pharmaceutical composition of claim 172, wherein the third domain comprises an amino acid sequence of SEQ ID NO: 383, 489, 647, 999, 1003, 1074, 1076, 1739, 3495, 3496, 3771, or 3809.

174. The pharmaceutical composition of claim 171, wherein the third domain comprises
(i) a single domain antibody that specifically binds to FLT3, and wherein the third domain comprises a CDR1 comprising an amino acid selected from the group consisting of SEQ ID NOS: 1080-1155 and 3497-3498, a CDR2 comprising an amino acid selected from the group consisting of SEQ ID NOS: 1156-1231 and 3499-3500, a CDR3 comprising an amino acid selected from the group consisting of SEQ ID NOS: 1232-1307 and 3501-3502;
(ii) a single domain antibody that specifically binds to PSMA, and wherein the third domain comprises a CDR1 comprising an amino acid sequence selected from the group consisting of SEQ ID NOS: 462-465, a CDR2 comprising an amino acid sequence selected from the group consisting of SEQ ID NOS: 466-472, and a CDR3 comprising an amino acid sequence selected from the group consisting of SEQ ID NOS: 474-475;
(iii) a single domain antibody that specifically binds to MSLN, and wherein the third domain comprises a CDR1 comprising an amino acid sequence selected from the group consisting of SEQ ID NOS: 490-528, a CDR2 comprising an amino acid selected from the group consisting of SEQ ID NOS: 529-567, and a CDR3 comprising an amino acid selected from the group consisting of SEQ ID NOS: 568-606;
(iv) a single domain antibody that specifically binds to BCMA, and wherein the third domain CDR1 comprises comprising an amino acid selected from the group consisting of SEQ ID NOS: 1-115, a CDR2 comprising an amino acid selected from the group consisting of SEQ ID NOS: 116-230, and a CDR3 comprising an amino acid selected from the group consisting of SEQ ID NOS: 231-345;
(v) a single domain antibody that specifically binds to DLL3, and wherein the third domain comprises a CDR1 comprising an amino acid selected from the group consisting of SEQ ID NOS: 1751-2193, a CDR2 comprising an amino acid selected from the group consisting of SEQ ID NOS: 2194-2636, and a CDR3 comprising an amino acid selected from the group consisting of SEQ ID NOS: 2637-3080;
(vi) a single domain antibody that specifically binds to EGFR, and wherein the third domain comprises a CDR1 comprising an amino acid selected from the group consisting of SEQ ID NOS: 651-699, a CDR2 comprising an amino acid selected from the group consisting of SEQ ID NOS: 700-748, a CDR3 comprising an amino acid selected from the group consisting of SEQ ID NOS: 479-797; or
(vii) a single domain antibody that specifically binds to EpCAM, and wherein the third domain comprises a CDR1 comprising an amino acid selected from the group consisting of SEQ ID NOS: 847-884, a CDR2 comprising an amino acid selected from the group consisting of SEQ ID NOS: 885-922, a CDR3 comprising an amino acid selected from the group consisting of SEQ ID NOS: 923-960.

175. The pharmaceutical composition of claim 171, wherein the half-life extended immune cell engaging protein comprises an amino acid sequence having at least 80% homology to any one of SEQ ID NOS: 3824-3831, 3833-3837, and 3839-3858.

176. The pharmaceutical composition of claim 171, wherein the half-life extended immune cell engaging protein comprises an amino acid sequence of any one of SEQ ID NOS: 3824-3831, 3833-3837, and 3839-3858.

177. The pharmaceutical composition of claim 155, wherein linkers L1 and L2 are each, independently, (GS)n (SEQ ID NO: 3859), (GGS)n (SEQ ID NO: 3860), (GGGS)n (SEQ ID NO: 3861), (GGSG)n (SEQ ID NO: 3862), (GGSGG)n (SEQ ID NO: 3863), (GGGGS)n (SEQ ID NO: 3864), (GGGGG)n (SEQ ID NO: 3865), or (GGG)n (SEQ ID NO: 3866), wherein n is 1, 2, 3, 4, 5, 6, 7, 8, 9, or 10, (GGGGSGGGGSGGGGSGGGGS)(SEQ ID NO: 3867), (GGGGSGGGGSGGGGS) (SEQ ID NO: 3868), LPETG (SEQ ID NO: 3869), (GGGGSGGGS) (SEQ ID NO: 3871) or SGGG (SEQ ID NO: 3872).

178. The pharmaceutical composition of claim 155, wherein
(i) the extended-release binding protein has a higher therapeutic index than a corresponding half-life-extended immune cell engaging protein without the masking peptide;
(ii) administration of the extended-release binding protein results in a lower Cmax/Cmin ratio of an active version of the extended-release binding protein in systemic circulation than the Cmax/Cmin ratio when a corresponding half-life extended immune cell engaging protein without the masking peptide is administered;
(iii) multiple administration of the extended-release binding protein results in a more gradual increase of a level of an active version of the extended-release binding protein in systemic circulation than when a corresponding half-life-extended immune cell engaging protein without the masking peptide is administered; or
(iv) administration of the extended-release binding protein results in a lower Cytokine release syndrome (CRS) level than the CRS observed when a corresponding half-life extended immune cell engaging protein without the masking peptide is administered.

179. A method for treatment or amelioration of a disease, comprising administrating to a subject in need thereof a pharmaceutical composition of claim 155.

180. The method of claim 179, wherein the disease is cancer.
